(19) United States
(12) Patent Application Publication
(10) Pub. No.: US 2024/0408143 A1
(43) Pub. Date: Dec. 12, 2024

(54) METHODS FOR CULTURING MESENCHYMAL STEM CELLS, COMPOSITIONS AND IMPLEMENTATIONS THEREOF

(71) Applicant: Pandorum Technologies Private Limited, Bangalore (IN)

(72) Inventors: Tuhin BHOWMICK, New York, NY (US); Arun CHANDRU, Bangalore (IN); Midhun Ben THOMAS, Bangalore (IN); Suman SENGUPTA, Bangalore (IN); Deepthi MENON, Bangalore (IN); Wenson Rajan David KARUNAKARAN, Bangalore (IN); Shivaram SELVAM, Bangalore (IN)

(21) Appl. No.: 18/437,857

(22) Filed: Feb. 9, 2024

Related U.S. Application Data
(63) Continuation of application No. PCT/IN2022/050720, filed on Aug. 11, 2022.

(30) Foreign Application Priority Data
Aug. 11, 2021 (IN) 202141036331

(51) Int. Cl. A61K 35/28; A61P 27/02; C12N 5/0775

(52) U.S. Cl. CPC A61K 35/28; A61P 27/02; C12N 5/0663; C12N 2500/98; C12N 2501/60

(57) ABSTRACT

There is provided herein methods generating a population of primed mesenchymal stem cell-derived exosomes. A method in accordance with the disclosure may comprise expanding a population of mesenchymal stem cells (MSCs) in culture, administering one or more priming agents in the culture to prime the population of MSCs and obtain a population of primed MSCs: growing the population of die primed MCSs in culture to produce a primed-MSC-derived conditioned medium; collecting the primed MSC-conditioned medium; and purifying a population of exosomes from the primed MSC-conditioned medium. The one or more priming p agents may comprise a conditioned media derived from a population of stem cells different from the population of MSCs, a Nrf2 activator, or a combination thereof. There is also provided herein methods of treating tissues such as cornea and liver using the population of exosomes from the primed MSC-conditioned medium.

### CROSS-REFERENCE TO RELATED APPLICATION

This application claims priority to Indian Application No. 202141036331, filed on Aug. 11, 2021. All applications are hereby incorporated by reference in their entirety.

### BACKGROUND

There are several compelling preclinical and clinical studies demonstrating the efficacy of mesenchymal stem cells (MSC) and cell-free therapy using cell-derived exosomes in treating fibrosis, inflammation and promoting wound healing and tissue regeneration. The therapeutic effects of MSCs have been largely attributed to paracrine factors secreted by the cells including exosomes.

MSCs secreting exosomes perform as mediators of cell to cell communication, for example in but not limited to tumors, and play several roles in tumorigenesis, angiogenesis, metastasis and intracellular communication. Exosomes are nanoscale extracellular vesicles (EVs) that act as mediators of crosstalk between cells. MSC-derived exosomes (MSC-Exo) contain as cargo proteins such as growth factors, cytokines, lipid moieties, and nucleic acids including miRNA, mRNAs, and transfer RNA (tRNA)) and other non-coding RNAs (ncRNA) that may provide anti-fibrotic, anti-inflammatory and pro-regeneration therapeutic effects of exosomes in humans. MSC-Exo have also been found to activate several signaling pathways important in tissue regeneration and inflammation (Akt. ERK, and STAT3) as well as inducing the expression of numerous growth factors. While MSCs themselves can be used a therapeutic agents, advantages of using MSC-Exo over MSCs includes low immunogenicity, low risk of rejection and low tumorigenicity. MSC Exo are also less likely to suffer the pulmonary first-pass effect, which is important both for safety and for system-wide delivery to take place. Accordingly, exosome derived from MSCs have been attempted in the treatment of certain diseases and defects.

However, the complement of exosome cargo is affected by priming agents in culture that prime the MSCs and affect their activity and transcriptional profile. As a result, priming can dramatically affect, for good or ill and in unexpected ways, the therapeutic efficacy of exosomes produced by a given MSC population.

In addition, MSC-Exo are harvested from MSCs secreting the MSC-Exo. Expansion of MSCs to large quantities is one of the perquisites of MSC-Exo-based therapies. However, conventional methods that are available in the art do not allow for sufficient scale-up of production of MSCs and its secreted products for therapeutic applications at commercial scale.

There is therefore a need to identify priming agents and related MSC culturing methods for producing primed MSCs and the MSC-Exo produced thereby that could provide an adequate supply of exosomes with high and improved therapeutic efficacy.

### SUMMARY OF THE INVENTION

There is provided herein embodiments of a method of generating a population of primed mesenchymal stem cell-derived exosomes, the method comprising: (a) expanding a population of mesenchymal stein cells (MSCs) in culture; (b) priming the population of MSCs with a cell-derived conditioned medium derived from a population of cells different from the population of MSCs and at least one defined priming agent to obtain a population of primed MSCs; (c) growing the population of the primed MCSs in culture to produce a primed-MSC-derived conditioned medium; and (d) collecting the primed MSC-conditioned medium. In some variations, the method comprises (e) purifying the exosomes from the primed MSC-conditioned medium.

In some variations, the priming of the population of MSCs comprises: (1) contacting the population of MSCs with the cell-derived conditioned medium; and (2) contacting the population of MSCs with the at least one defined priming agent. Optionally, the population of MSCs are in contact with the cell-derived conditioned media from seeding until between about 60% and about 90% confluency, and the population of MSCs are contacted with the at least one defined priming agent starting from between about 60% and about 90% confluency. Optionally, the population of MSCs are in contact with the cell-derived conditioned media from seeding until between about 60% and about 90% confluency, and are in contact the at least one defined priming agent thereafter. Optionally, the population of MSCs are in contact with at least one defined priming agent for between about 12 hours and about 72 hours.

In some variations, the cell-derived conditioned medium from the different population of cells is a corneal stromal stem cell-derived conditioned medium.

In some variations, the at least one defined priming agent is a nuclear factor erythroid 2-related factor 2 (Nrf2) activator, a silencing information regulator factor 1 (SIRT1) activator, or an all-trans retinoic Acid (ATRA). Optionally, the at least one priming agent is the Nrf2 activator. Optionally, the Nrf2 activator is dimethyl fumarate (DMF) or 4 Octyl itaconate (4-OT).

In some variations, the population of MSCs is a population of bone marrow-derived MSCs (BM-MSCs), a population of umbilical cord-derived MSCs (UM-MSCs), a population of induced pluripotent stem cell (iPSC)-derived MSCs (iPSC-MSCs), or a population of Wharton's Jelly-derived MSCs (WJ-MSCs). Optionally, the population of BM-MSCs is a population of human BM-MSCs.

In some variations, the cell-derived conditioned medium is the corneal stem cell-derived conditioned medium, and the defined priming agent is DMF or 4 Octyl itaconate (4-OI). Optionally, the corneal stem cell-derived conditioned medium is present at a concentration of between about 10% and about 30%. Optionally, the DMF is present at a concentration of between about 50 μM and about 100 μM.

There is also provided herein embodiments of a population of primed MSC-derived exosomes produced with an embodiment of the method above.

There is also provided herein embodiments of a population of primed MSC-derived exosomes that are characterized by having one or more of, as compared to unprimed MSC-derived exosomes: (a) a lower expression level of vascular endothelial growth factor (VEGF); and (b) a higher expression level of nerve growth factor (NGF).Optionally, the primed MSC-derived exosomes, compared to the unprimed mesenchymal stem cell derived-exosomes, are characterized by one or more of: (c) a higher expression level of hepatic growth factor (HGF); and (d) a higher expression level of sFLT1. Optionally, the primed MSC-derived exosomes, compared to the unprimed mesenchymal stem cell derived-exosomes, are characterized by one or a combination of two or more of, three or more of, or all of: (a) at least 2× higher expression level of sFLT1; (b) an expression level of VEGF that is a quarter or less of the expression in unprimed MSC-derived exosomes; (c) at least 2× higher expression level of HGF; and (d) at least 3× higher expression of NGF. In some variations, the primed MSCs are prepared by: (1) contacting a population of MSCs with a corneal stein cell-derived conditioned medium; and (2) contacting the population of MSCs with an Nrf2 activator. Optionally, the Nrf2 activator is DMF or 4 Octyl itaconate (4-OI). Optionally, the population of MSCs are in contact with the corneal stem cell-derived conditioned media from seeding until between about 60% and about 90% confluency, and the population of MSCs are contacted with the Nrf2 activator starting from between about 60% and about 90% confluency. Optionally, the population of MSCs arm in contact with the Nrf2 activator for between about 12 hours and about 72 hours.

There is also provide herein embodiments of a method of treating a corneal defect, the method comprising administering to a corneal surface having the corneal defect a therapeutic dose of the population of exosomes disclosed herein. Optionally, the corneal defect is selected from the group consisting of: corneal scarring, keratitis, corneal ulcer, corneal abrasion, corneal epithelial damage, corneal stromal damage, infection-based corneal damage, trachoma, keratoconus, corneal perforation, corneal limbal injury, corneal dystrophy, neovascularization, vernal keratoconjunctivitis and dry eye. In some variations, the population of exosomes are comprised in an ophthalmic composition formulated for application on the corneal surface. Optionally, the composition is an eye drop liquid. Optionally, the eye drop liquid comprises a biocompatible polymer. Optionally, the biocompatible polymer is cross-linkable, and the method comprises administering to the corneal surface the eye drop liquid and crosslinking a sufficient portion of the cross-linkable polymers so that the eye drop liquid is converted to a hydrogel.

There is also provided herein embodiments of an ophthalmic composition formulated for application on a corneal surface and comprising a populations of exosomes as disclosed herein. Optionally, the ophthalmic composition is in an eye drop liquid. Optionally, the eye drop liquid comprises a biocompatible polymer. Optionally, the ophthalmic composition is a hydrogel and at least a portion of the biocompatible polymers are cross-linked.

There is also provided herein embodiments of a method of generating a population of primed mesenchymal stem cell-derived exosomes, the method comprising: (a) culturing a population of mesenchymal stem cells (MSCs) in a culture medium; (b) priming the population of MSCs with an Nrf2 activator to obtain a population of primed MSCs; (c) growing the population of the primed MCSs in a collection medium, wherein the collection medium becomes enriched with exosomes produced by the primed MSCs, thereby producing a primed-MSC-derived conditioned medium; and (d) collecting the primed MSC-conditioned medium. Optionally, the method further comprises (e) purifying the exosomes from the primed MSC-conditioned medium. Optionally, the population of MSCs are grown in a first culture medium from seeding until between about 60% and about 90% confluency, then contacted with the Nrf2 activator. Optionally, the population of MSCs are in contact with the Nrf2 activator for between about 12 hours and 72 hours. Optionally, the Nrf2 activator is dimethyl fumarate (DMF) or 4 Octyl itaconate (4-OI). Optionally. DMF is present at a concentration of between about 50 μM and about 100 μM. Optionally, the population of MSCs is a population of bone marrow MSCs (BM-MSCs), a population of umbilical cord-derived MSCs (UM-MSCs), a population of induced pluripotent stem cell (iPSC)-derived MSCs (iPSC-MSCs) or a population of Wharton's Jelly-derived MSCs (WJ-MSCs).

There is also provided herein embodiments of a population of primed MSC-derived exosomes produced with a method provided above.

There is also provided a population of primed MSC-derived exosomes that are characterized by having one or more of, as compared to unprimed MSC-derived exosomes: (a) a higher expression level of hepatic growth factor (HGF); and (b) a higher expression level of nerve growth factor (NGF). Optionally, the primed MSC-derived exosomes, compared to the unprimed MSC derived-exosomes, are characterized by: (a) a higher expression level of HGF; and (b) a higher expression level of NGF. Optionally, the primed MSC-derived exosomes, compared to the unprimed MSC derived-exosomes, are characterized by one or both of: (a) at least 1.2× higher expression level of HGF; and (b) at least 2× higher expression level of NGF.

In some variations, the primed MSCs are prepared by: (1) growing a population of MSCs in a first culture medium; and (2) contacting the population of MSCs with an Nrf2 activator. Optionally, the Nrf2 activator is DMF or 4 Octyl itaconate (4-OI). Optionally, the DMF is present at a concentration of between about 50 μM and about 100 μM. Optionally, the MSCs are grown in the first culture medium from seeding until between about 60% and about 90% confluency, then contacted with the Nrf2 activator. Optionally, the population of MSCs are in contact with the Nrf2 activator for between about 12 hours and about 72 hours, then exchanged with a collection medium.

There is also provided herein embodiments, of a method of treating a liver condition, the method comprising administering to a subject having the liver condition a therapeutic amount of the population of exosomes provided herein above. Optionally, the liver condition is a non-alcoholic fatty liver disease (NAFLD). Optionally, the NAFLD is a non-alcoholic fatty liver (NAFL) or a non-alcoholic steatohepatitis (NASH). Optionally, the population of exosomes is administered to the liver through an intravenous route. Optionally, intravenous mute is through a hepatic portal vein.

There is also provided herein embodiments of a composition comprising the population of exosomes as disclosed herein above. Optionally, the composition is for use in the treatment of a liver condition. Optionally, the liver condition is a non-alcoholic fatty liver disease (NAFLD). Optionally, the NAFLD is a non-alcoholic fatty liver (NAFL) or a non-alcoholic steatohepatitis (NASH).

There is also provided herein embodiments of a method of increasing the secretion of exosomes by a population of mesenchymal stem cells (MSCs), the method comprising: (a) culturing a population of MSCs in a culture medium; (b) priming the population of MSCs with an Nrf2 activator to obtain a population of primed MSCs; and (c) growing the population of the primed MCSs in a collection medium, wherein the collection medium becomes enriched with exosomes produced by the primed MSCs. Optionally, the population of MSCs are grown in a first culture medium from seeding until between about 60% and about 90% confluency, then contacted with the Nrf2 activator. Optionally, the population of MSCs is in contact with the Nrf2 activator for between about 12 hours and 72 hours. Optionally, the Nrf2 activator is dimethyl fumarate (DMF) or 4 Octyl itaconate (4-OI).Optionally, the DMF is present at a concentration of between about 50 μM and about 100 μM. Optionally, the population of MSCs is a population of bone marrow MSCs (BM-MSCs), a population of umbilical cord-derived MSCs (UM-MSCs), a population of induced pluripotent stem cell (iPSC)-derived MSCs (iPSC-MSCs) or a population of Wharton's Jelly-derived MSCs (WJ-MSCs).

These and other features, aspects, and advantages of the present subject matter will be better understood with reference to the following description and appended claims. This summary is provided to introduce a selection of concepts in a simplified form. This summary is not intended to identify key features or essential features of the claimed subject matter, nor is it intended to be used to limit the scope of the claimed subject matter.

### BRIEF DESCRIPTION OF THE DRAWINGS

The following drawings form a part of the present specification and are included to further illustrate aspects of the present disclosure. The disclosure may be better understood by reference to the drawings in combination with the detailed description of the specific embodiments presented herein.

FIGS. 1A-1E depicts bar graphs of quantification of the secretome of human bone marrow-derived MSCs (hBM-MSCs) primed with corneal stromal stem cell-derived conditioned media (CSSC-CM). The quantification was based on enzyme-linked immunoassay (ELISA) of the culture medium or a fraction thereof in which the hBM-MSC was grown, hBM-MSC cells were primed with CSSC-CM (10% of 20% of the culture media was replaced with the CSSC-CM) and secretome profile of primed hBM-MSC cells was characterized, in particular the secreted level of HGF (FIG. 1A), VEGF (FIG. 1B), sFLT1 (FIG. 1C), IL-6 (FIG. 1D), NGF (FIG. 1E), in accordance with an embodiment of the present disclosure.

FIGS. 2A-2E depicts bar graphs of the quantification of the anti-inflammatory effect of primed hBM-MSCs derived exosomes on RAW 264.7 macrophage cells were stimulated with lipopolysaccharide (LPS) binding protein in the presence of indicated primed exosomes. Cytokine expression was measured by ELISA for IL-6 (FIG. 2A), IL-1β (FIG. 2B), IL-10 (FIG. 2C), TNF-α (FIG. 2D), and IFNγ (FIG. 2E), in accordance with an embodiment of the present disclosure.

FIGS. 3A-3E depicts bar graphs of the quantification of cytokine expression from RAW 264.7 macrophage cells stimulated with LPS in the presence of indicated primed exosomes (0.5 billion exosomes). Cytokine expression was measured by quantitative PCT (qPCR) for IL-6 (FIG. 3A), IL-1β (FIG. 3B), TNF-α (FIG. 3C), IL-10 (FIG. 3D) and IFNγ (FIG. 3E) respectively.

FIGS. 4A-4F depicts representative immunofluorescence images of the characterization of anti-fibrosis properties of different exosome variants on human dermal fibroblasts treated with TGF-β. Human Dermal Fibroblasts (HDFs) were co-treated with TGF-β (FIG. 4B) and indicated exosomes (FIGS. 4C-4F) for 24 hours. Cells were fixed and α-SMA expression was assessed by immunostaining. TGF-β induced α-SMA expression in TGF-β only treated cells, which was blocked in the presence of CSSC-primed BM-MSC exosomes (FIGS. 4D-4E) and CSSC-exosomes (FIG. 4F) and to a lesser extent by naïve hBM-MSC-exosomes (FIG. 4C) in accordance with an embodiment of the present disclosure.

FIG. 5 depicts a bar graph showing an effect of the priming of BM-MSCs mediated by Nrf2 activator (DMF or 4-OI), and characterizes the secretome and exosomes profiles under difference priming conditions. Data is representative of 3 technical replicates ±SEM. NRF2 activator DMF shows higher yield of exosomes, in accordance with an embodiment of the present disclosure.

FIGS. 6A-6F depicts bar graphs of the quantification of the secretome marker profiling of primed cells under different priming conditions. The secretion of HGF (FIG. 6A), IL-6 (FIG. 6B), VEGF (FIG. 6C), sFLT1 (FIG. 6D), NGF (FIG. 6E) and SDF-1 (FIG. 6F) were quantified at the protein level by ELISA. Expression of HGF (FIG. 6A) was higher in all primed variants, while VEGF and sFLT1 levels were unaltered (FIGS. 6C-6D). Curcumin and Nrf2 activators attenuated the expression of IL-6 (FIG. 6B), whereas NGF secretory levels were enhanced by Nrf2 activators 4-OI and DMF (FIG. 6E). The combination of curcumin-conditioned media+DMF did not appear to have a pronounced effect on any of the readouts, in accordance with an embodiment of the present disclosure.

FIGS. 7A-7E depicts bar graphs of quantifications profiling the cargo (such as exosomal proteins) of exosomes secreted by MSCs under priming with different priming agents such as Curcumin (CUR) and Nrf2 activator DM and 4-OI. Quantified cargo include exosomal HGF (FIG. 7A), exosomal VEGF (FIG. 7B), exosomal sFLT1 (FIG. 7C), exosomal NGF (FIG. 7D), exosomal TGF-β (FIG. 7E), and exosomal SDF-1 (FIG. 7F). Exosomal HGF was higher in all primed variants compared to naïve MSCs. Expression of exosomal NGF was induced with Nrf2 activator priming (especially with DMF), sFLT1, TGF-β and SDF-1 levels remained unchanged.

FIGS. 8A-8E depicts bar graphs of the quantification of the anti-inflammatory effect of primed hBM-MSC-derived exosomes (hBM-MSC-Exo) on RAW 264.7 macrophage cells, RAW 264.7 macrophage cells were stimulated with LPS in the presence of indicated primed exosomes (0.4 billion exosomes). Cytokine expression was measured by ELISA for IL-6 (FIG. 8A), IL-1β (FIG. 8B), IL-10 (FIG. 8C), TNF-α (FIG. 8D), and IFNγ (FIG. 8E), in accordance with an embodiment of the present disclosure.

FIGS. 9A-9D depicts bar graphs of the quantification of exosomal cargo proteins of exosomes derived from hBM-MSC combinatorically primed with CSSC-CM and Nrf2 activator. Quantification was based on ELISA applied to purified exosomes produced in accordance with an embodiment of the present disclosure. Exosomal HGF (FIG. 9A), exosomal VEGF (FIG. 9B), exosomal sFLT1 (FIG. 9C), and exosomal NGF (FIG. 9D) were quantified at the protein level by ELISA.

FIGS. 10A-10E depicts bar graphs of the quantification of the characterization of anti-inflammatory activity of different exosome variants primed with CSSC-CM with Nrf2 activator (DMF). RAW 264.7 macrophage cells were stimulated with LPS in the presence of indicated primed exosomes (about 0.5 billion exosomes). Cytokine expression was measured by ELISA for IL-6 (FIG. 10A), IL-1β (FIG. 10B), TNF-α (FIG. 10C), IL-10 (FIG. 10D) and IFNγ (FIG. 10E) respectively in accordance with an embodiment of the present disclosure.

FIGS. 11A-11F depicts representative immunofluorescence images of the characterization of anti-fibrotic activity of different exosome variants from BM-MSCs primed with CSSC-CM and/or the Nrf2 activator (DMF) on human dermal fibroblasts treated with TGF-β. The human dermal fibroblasts were treated with TGF-β and indicated exosome variants for 24 hours and probed for α-SMA expression. TGF-β induced α-SMA in the TGF-β only treated cells (FIG. 11B) while exosomes inhibited the induction of a-SMA to different extents as shown above.

FIG. 12 depicts representative images of the characterization of wound healing activity of different exosome variants, hBM-MSCs primed with CSSC-CM and/or Nrf2 activator (DMF) observed across multiple time points. The representative images depicting the time course of the wound closure (2D scratch assay) on a monolayer of epithelial cells were observed across the multiple time points of 0 hours, 24 hours, 48 hours, and 72 hours. BM-MSCs; CSSC-CM (20%); Nrf2 activator: DMF, in accordance with an embodiment of the present disclosure.

FIG. 13 depicts a graph of a cell migration/cell proliferation assay tracking confluence of cells treated with different exosome variants including naïve exosomes, exosomes derived from hBM-MSCs primed with CSSC-CM, the Nrf2 activator, or the combination thereof.

FIG. 14 depicts representative images of a wound healing assay using rabbit corneas having an open epithelial wound including an untreated control, a liquid cornea biopolymer, and a combination of the liquid cornea biopolymer and exosomes derived from hBM-MSCs primed with CSSC-CM and the Nrf2 activator. DMF.

FIG. 15A depicts representative immunofluorescence images stained for CYP34A in healthy liver spheroids, NASH-induced liver spheroids, or exosome treated NASH-induced liver spheroids.

FIG. 15B illustrates a bar graph of the quantification of secreted albumin levels in human liver spheroids including NASH-induced liver spheroids and exosome treated NASH-induced liver spheroids after 24 hours, 48 hours, and 72 hours.

FIG. 15C depicts representative immunofluorescence images stained for collagen, a fibrosis marker, in NASH-induced liver spheroids and exosome treated liver spheroids.

FIG. 15D illustrates a bar graph of the quantification of percent coverage of collagen deposition in human liver spheroids including NASH-induced liver spheroids and exosome treated NASH-induced liver spheroids.

FIG. 15E depicts representative immunofluorescence images stained for α-SMA, a fibrosis marker, in healthy liver spheroids, NASH-induced liver spheroids, and exosome treated NASH-induced liver spheroids.

FIG. 15F illustrates a bar graph of the quantification of intensity of α-SMA in human liver spheroids including NASH-induced liver spheroids or exosome treated NASH-induced liver spheroids.

FIG. 16A depicts a gene expression heatmap of global gene expression profiles of NASH-induced liver spheroids treated with primed exosomes derived from BM-MSCs.

FIG. 16B depicts a principal component analysis plot comparing differentially expressed genes of NASH-induced liver spheroids treated with primed exosomes.

FIG. 17A depicts a gene expression heatmap of liver specific genes of profiles of NASH-induced liver spheroids treated with naïve exosome or primed exosomes.

FIG. 17B depicts a gene expression heatmap of non-alcoholic steatohepatitis (NASH)/fibrosis related genes of profiles of NASH-induced liver spheroids treated with naïve exosome or primed exosomes.

FIG. 17C depicts a gene expression heatmap of stellate cell specific genes of profiles of NASH-induced liver spheroids treated with naïve exosome or primed exosomes.

FIG. 17D depicts a gene expression heatmap of genes related to xenobiotic metabolic process of profiles of NASH-induced liver spheroids treated with naïve exosome or primed exosomes.

FIG. 17E depicts a gene expression heatmap of fatty acid metabolism genes of profiles of NASH-induced liver spheroids treated with naïve exosome or primed exosomes.

FIG. 17F depicts a gene expression heatmap of genes related to the Epoxygenase p450 pathway of profiles of NASH-induced liver spheroids treated with naïve exosome or primed exosomes.

FIG. 17G depicts a gene expression heatmap of steato-fibrotic related genes of profiles of NASH-induced liver spheroids treated with naïve exosome or primed exosomes.

FIG. 18A depicts a plot based on the Jaccard Similarity Index for NASH/fibrosis related genes, global genes, and liver specific genes in liver spheroids.

FIG. 18B depicts a plot based on the Jaccard Similarity Index for genes related to neurogenesis, angiogenesis, and inflammatory response genes in liver spheroids.

FIG. 18C depicts a plot based on the Jaccard Similarity Index for genes related to the extracellular matrix, wound healing, and tissue remodelling in liver spheroids.

FIG. 19 depicts a gene expression heatmap of genes related to enriched biological processes in untreated, NASH-induced, or primed exosome treated NASH-induced liver spheroids.

FIG. 20 depicts a flow chart of a method for generating primed exosomes in accordance with embodiments of the disclosure.

### DETAILED DESCRIPTION

Those skilled in the art will be aware that the present disclosure is subject to variations and modifications other than those specifically described. It is to be understood that the present disclosure includes all such variations and modifications. The disclosure also includes all such steps, features, compositions, and compounds referred to or indicated in this specification, individually or collectively, and any or all combinations of any or more of such steps or features.

### Definitions

For convenience, before further description of the present disclosure, certain terms employed in the specification, and examples are delineated here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”.

Throughout this specification, unless the context requires otherwise the word “comprise”, and variations such as “comprises” and “comprising”, will be understood to imply the inclusion of a stated element or step or group of element or steps but not the exclusion of any other element or step or group of element or steps.

The term “including” is used to mean “including but not limited to”. “Including” and “including but not limited to” are used interchangeably.

For the purposes of the present document, the term “a population of expanded primed mesenchymal stem cells” refers to the population of mesenchymal stem cells which has an increased number of cells as compared to the population of mesenchymal stem cells obtained initially for culturing. The culturing process does not differentiate the cells, it just increases the number of cells manifolds. In addition, priming refers to the use of small molecule priming agents.

The term “three-dimensional culture” or “3D culture” refers to a system of culturing the cells in-vitro in which the biological cells are allowed to grow and interact with their surroundings in all the three dimensions.

The term “two-dimensional culture” or “2D culture” refers to the method of culturing the cells as a monolayer on a surface by which the biological cells are able to interact with their surroundings in two dimensions.

The term “spheroid-based system” refers to the process of culturing mesenchymal stem cells (MSC) in a three-dimensional manner by formation of spheroids according to the method as described in the present disclosure.

The term “microcarrier-based system” refers to the process of culturing mesenchymal stem cells (MSC) in a three-dimensional manner by the formation of alginate-gelatin (Alg/Gel) microcarriers or microbeads according to the method as described in the present disclosure.

The term “microcarriers” and “microbeads” are used interchangeably: it refers to the alginate-gelatin (Alg/Gel) microcarriers or microbeads as described in the present disclosure.

The term “mesenchymal stem cell derived-conditioned medium or “MSC-CM” refers to the medium obtained after the growth of the MSC. The conditioned medium thus obtained comprises secreted cell modulators and multiple factors critical for tissue regeneration. The conditioned medium thus obtained also comprises secretome, and exosomes which needs to be purified from the conditioned medium before being able to apply for therapeutic purposes. The process for obtaining expanded MSC as described herein also leads to the formation of MSC-CM, therefore, it can be said that a single process leads to the procurement of a population of expanded primed MSC as well as of MSC-CM.

The term “exosomes” refers to cell-secreted extracellular vesicle in the nanoscale range (such as in the 20-200 nm range) that contain as cargo biological molecules such as protein, DNA, and RNA (including various types such as mRNA and miRNA), generally from the biological cells that secretes them. Some of the biological molecules may have anti-inflammatory, anti-fibrotic and regenerative properties, and may be of clinical interest.

The term “micromolecules” or “small molecules” are defined as synthetic or naturally occurring chemical modifiers of cell behavior and induces therapeutic characteristics. The small molecules have a molecular weight of less than 800 Da.

The term “macromolecules” are biological agents having a molecular weight of more than 800 Da. In the present disclosure, the macromolecule includes proteins, lipids, nucleic acids, growth factors, cytokines, and components of conditioned media.

For the purposes of the present document, the term “corneal limbal stem cells” refers to the population of stem cells which reside in the corneal limbal stem cell niche. The corneal limbal stem cell is referred to population of stem cells represented primarily by corneal stromal stem cells (CSSC), and limbal epithelial stem cells (LESC).

The term “corneal stromal stem cell derived-conditioned medium” or “CSSC-CM” refers to the medium in which corneal stromal stein cells (CSSC) are grown. The CSSC-CM as described herein is obtained by culturing of CSSC in a manner known in the art or by culturing of CSSC as per a method disclosed herein.

The term “xeno-free” as described in the present disclosure refers to a media which is free of any product which is derived from non-human animal. The method being xeno-free is an important advantage because of its plausibility of clinical application.

The term “subject” refers to an animal subject that may be administered with a therapeutic agent, for example a composition comprising exosomes. The animal subject may be a mammalian subject. The mammalian subject may be one who is suffering from, or was diagnosed with, a condition as mentioned in the present disclosure. The mammalian subject may be a human subject.

The term “therapeutically effective amount” refers to the amount of a composition which is required for treating the conditions of a subject.

The term “naïve cells” here refer to the unprimed mesenchymal stems are not primed with any conditioned medium. Therefore, the terms unprimed and naïve are interchangeably used in the present disclosure.

Despite great variability of MSC due to different in-vitro cell culture methods, there are various limitations associated with the conventional methods that have limited the success of MSC therapy in clinical trials. The high sensitivity of MSC to the harsh microenvironment of immune-mediated, inflammatory, and degenerative diseases is still a great obstacle for successful MSC-based therapies. Inhospitable tissue surroundings are able to limit the functions and survival of transplanted MSC. Further, the use of homogenous population of MSCs limited their use in therapeutic applications. Many other limitations have also jeopardized MSC-based therapies, such as cell senescence due to in vitro overexpansion, function loss after cryopreservation, and inconsistency of in-vivo therapeutic effects among pre-clinical and clinical trials.

In order to address the problems faced in the art, the present disclosure, one aspect of the disclosure provides methods for production of mesenchymal stem cells (MSCs) and production of exosomes purified from the MSCs. In some variations, methods provided in the present disclosure includes isolating a sub-population of mesenchymal stem cells from various sources that expresses a signature set of markers. In some variations, the sub-population of mesenchymal stem cells may be further modified using hTERT (human telomerase reverse transcriptase) that can extend the doubling potential of engineered MSCs (eMSCs) to facilitate scalable and homogeneous production of cells and therapeutic exosomes derived from said MSCs.

Another aspect of the methods of the present disclosure is the priming of the MSCs with one or more priming agents such as small molecule and macromolecules, and/or conditioned media derived from other stem cell populations. The cells and exosomes derived from the methods of the present disclosure may be used as such or in combination with each other for clinical applications. In some variations, conditioned media from a population of naïve MSCs may be used to prime a different population of naïve MSCs from a different tissue source. In some variations, the use of two or more priming agents, i.e. combinatorial priming strategies, may enhance one or more of regenerative, anti-inflammatory, and anti-fibrotic properties of the MSCs. and/or exosomes secreted by the MSCs. For convenience of presentation, exosomes secreted by naïve MSCs may be referred to herein as “naïve exosomes” and exosomes secreted by MSCs that are primed, for example with one or more priming agents and/or conditioned media from other cells, may be referred to herein as “primed exosomes” or “primed exosome variants”. Naïve or primed exosomes in addition to naïve/primed MSCs may be used as such or in combination thereof for the therapeutic applications, and different cell-based therapies to address multiple unmet clinical needs.

The present disclosure refers to the in-vitro culture of umbilical cord blood derived mesenchymal stem cells (UC-MSCs)/Wharton Jelly derived MSCs (WJ-MSCs)/bone marrow derived MCSs (BM-MSCs) and the subsequent selection of a unique sub-population that possesses enriched factors related to one or more of anti-fibrotic, anti-inflammatory/immunomodulatory and pro-angiogenic activities. In order to perform said method, exosomes may be isolated from a defined MSCs population and characterized comprehensively. The disclosure describes the protocol/methodology of priming various MSC populations, such as UC-MSCs, WJ-MSCs, and BM-MSCs with different priming agents (in some instances clinically approved priming agents), including, but not limited to Nrf2 activators, SRT1 activators, ATRA, conditioned medium, alone or in combinations thereof, to enhance regenerative, stemness and anti-inflammatory properties of the cells. Single priming or combinatorial priming may be used to generate different exosome variants with specific/enriched cargo loaded factors. In some variations, the exosome variants may be characterized both at the physical and molecular level for their functional efficacy. In some variations, the exosome variants may be categorized based on their functionality for different inflammatory and fibrosis associated diseases such as pulmonary dysfunction, acute respiratory distress, inflammation associated disorders including but not limited to rheumatoid arthritis, systemic juvenile idiopathic arthritis, idiopathic pulmonary fibrosis, acute respiratory distress syndrome (ARDS), pneumonia, bronchitis, chronic obstructive pulmonary disease (COPD), COVID-19, coronavirus class of infection, cystic fibrosis, hantavirus, influenza, tuberculosis, systemic lupus, osteoarthritis, NASH, liver fibrosis, Mooren's ulcer, neurotrophic ulcer, myocardial infarction, etc.

In some variations, the priming agent may be one of the following: (a) Priming with an Nrf2 activator: Without being bound by theory, in some variations, priming with Nrf2 activator enhances the anti-inflammatory property of the primed MSCs and the exosomes secreted by the MSCs. The exosomes secreted by the primed MSCs (“primed exosomes) may be enriched with cargo that are advantageous for the treatment of inflammation associated disorders. Examples of inflammation-associate disorders include rheumatoid arthritis, systemic juvenile idiopathic arthritis, idiopathic pulmonary fibrosis, acute respiratory distress syndrome (ARDS), pneumonia, bronchitis, chronic obstructive pulmonary disease (COPD), COVID-19, coronavirus class of infection, cystic fibrosis, hantavirus, influenza, tuberculosis, systemic lupus, osteoarthritis, non-alcoholic fatty liver disorder (NAFLD) (which may be non-alcoholic fatty liver (NAFL) or non-alcoholic steatohepatitis (NASH)), liver fibrosis, Mooren's ulcer, neurotrophic ulcer, and myocardial infarction; (b) Priming with a SIRT 1 activator; (c) Combinatorial priming with an Nrf2 activator+a SIRT 1 activator: Without being bound by theory, in some variations, regenerative therapeutic efficacy is enhanced by priming the MSCs with a SRT1 activator as well as a Nrf2 activator. Enriched therapeutic grade exosomes can them be applied for vascular tissue regeneration. In some variations, combinatorial priming with a SRT1 activator and a Nrf2 activator may induce the MSCs to produce exosomes with enhanced therapeutic cargo-loaded factors with one or more of anti-inflammatory, anti-fibrotic, and pro-angiogenic effects; (d) Combinatorial priming with Nrf2 activators+CSSC-derived conditioned medium (CCSC-CM): In some variations, without being bound by theory, a combinatorial priming with a Nr2 activator such as DMF with CSSC-CM induces MSCs to produce exosomes that are enriched with anti-inflammatory factors but reduced in angiogenesis factors. Hence, the primed exosomes from MSCs combinatorically primed with an Nrf2 activator and CSSC-CM may be used for regeneration of avascular tissue such as comea; (c) NRF2 activators+SIRT 1 activators+all-trans retinoic acid+CSSC-CM. In addition, the induction of hypoxia via physical (create a hypoxic microenvironment) or chemical (HIF-1α) inducers in the priming process will increase the survivability and stemness of the primed MSCs. The present disclosure also discloses the protocol of combinatorial priming with SRT1 activator and Nrf2 activators in specified BM-MSCs/UC-MSCs/WJ-MSCs in presence and absence of hypoxia to generate more significant therapeutically enriched exosomes for the treatment of regenerative therapy in lung, liver, and bioengineering and 3D bioprinting of vascular tissue. An aim of the present disclosure is to enhance cell yield significantly and address a larger cohort of patients, keeping the same number of product manufacturing runs and downstream processing.

In some variations, the MSC populations may be modified using hTERT (human telomerase reverse transcriptase) that extend the doubling potential of engineered MSCs (eMSCs) to facilitate scalable and homogeneous production of cells and therapeutic exosomes. Modulating specific pathway in MSCs, eMSCs, or induced pluripotent stem cells (iPSCs) and/or applying various combinatorial priming protocols may be used to generate exosomes that are customized for downstream applications.

The present disclosure provides MSCs derived from various sources including human bone marrow, adipose tissue, umbilical cord, unrestricted somatic stem cells. Wharton jelly, dental pulp-derived MSCs, iPSCs, engineered cells, corneal limbal stem cells, as source materials. The MSCs may be grown, and optionally primed, to produce unique sub-populations or variants that expressing a signature set of markers and produce exosomes that are therapeutically effective for a given condition or set of conditions.

One aspect of the present disclosure lies in priming MSCs derived from various tissue sources, such as bone marrow, adipose, umbilical cord, etc., with specific combination of inducers to activate certain pathways for the production of therapeutic exosomes with enriched factors as cargo, including one or a combination of two or more of anti-inflammatory, anti-fibrosis, pro-wound healing, angiogenesis (pro-/anti-), and re-innervation factors, for regeneration of avascular or vascular tissues.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the disclosure, the preferred methods, and materials are now described. All publications mentioned herein are incorporated herein by reference.

The present disclosure is not to be limited in scope by the specific embodiments described herein, which are intended for the purposes of exemplification only. Functionally-equivalent products, compositions, and methods are clearly within the scope of the disclosure, as described herein.

### Methods for Generating Primed Mesenchymal Stem Cell-Derived Exosomes

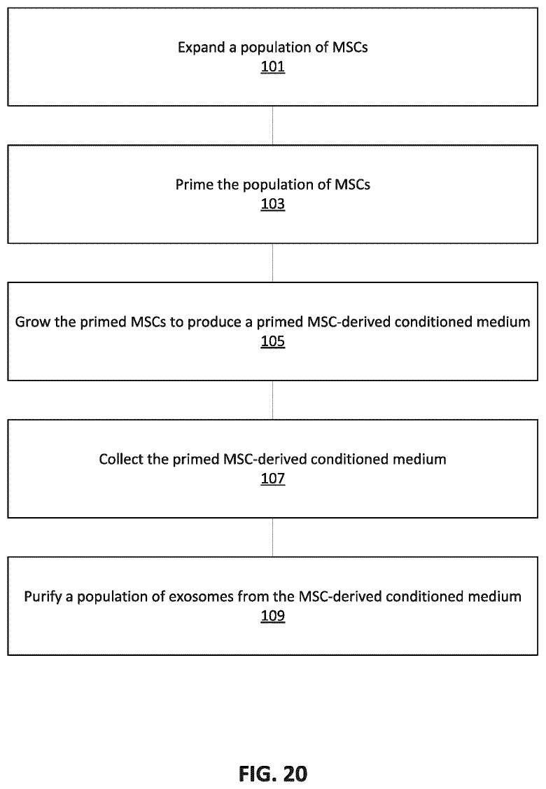

In some embodiments of the present disclosure, there is provided a method 100 of generating a population of primed mesenchymal stem cell-derived exosomes. FIG. 20 depicts a flowchart of an embodiment of the method 100. The method may comprise the following steps: step 101—expanding a population of mesenchymal stem cells (MSCs) in culture; step 103—administering one or more priming agents in the culture to prime the population of MSCs and obtain a population of primed MSCs; step 105—growing the population of the primed MCSs in culture to produce a primed-MSC-derived conditioned medium; and step 107—collecting the primed MSC-conditioned medium. In some variations, the method further comprises a step 109—purifying a population of exosomes from the primed MSC-conditioned medium.

### Types of MSC Populations

In some variations, the population of MSCs may be selected from the group consisting of bone marrow-derived mesenchymal stem cells, adipose-derived mesenchymal stem cells, umbilical cord-derived mesenchymal stem cells, Wharton's Jelly-derived mesenchymal stem cells, dental pulp-derived mesenchymal stem cells, induced pluripotent stem cell-derived mesenchymal stem cells, corneal limbal stem cells, and corneal stromal stem cells. In some variations, the MSCs may be primary cells, or engineered cells. In some variations, the MSCs may be freshy derived from a primary source or cryopreserved then defrosted.

In some variations, the MSCs may be a sub-population of stem cells expressing stem cell markers selected from the group consisting of CD34⁻, α-SMA⁻ CD46⁺, CD47⁺, CD73⁺, CD90⁺, CD105^{+/-}, CD54⁺, CD58⁺, CD106⁺, CD142^{+/-}, CD146⁺, CD166⁺, CD200⁺, CD273⁺, CD274⁺, CD276⁺, or a combination thereof. In some variations, the sub-population of stem cells may be isolated as follows: (a) selecting a first sub-population of mesenchymal stem cells from the sub-population of stem cell, wherein the first sub-population of mesenchymal stem cell expresses positive markers selected from the group consisting of CD34⁻, α-SMA⁻. CD73⁺; CD90⁺, and CD166⁺, and (b) selecting a second sub-population of mesenchymal stem cells from the sub-population of stem cell, wherein the second sub-population of mesenchymal stem cell expresses markers selected from the group consisting of CD146⁺, CD54⁺, CD58⁺ and CD142^{+/-}.

In some variations, the MSCs may comprise comprises a non-viral human telomerase enzyme reverse transcriptase (hTERT).

### Culture Media

In some variations, the MSCs may be expanded in a xeno-free culture medium. In some variations, the MSCs may be grown in hypoxic conditions, optionally having oxygen in the culture medium in the range of 0.2-10%. In some variations, the MSCs may be cultured a Minimum Essential Medium and at least one type of collagenase enzyme in the range of 5-20 IU/μ1 to obtain the expanded stem cells, wherein the at least one type of collagenase enzyme is a combination of collagenase-I and collagenase-II.

### Priming Agents

In some embodiments, a priming agent may be applied to the MSCs to change the cell's activity or gene expression pattern. The change induced in the MSCs by the priming agent may result in, or induce, changes in one or more characteristics, such as the presence of certain biomolecules or the relative expression of certain biomolecules in the cargo, of the exosomes produced and secreted by the affected MSCs. The priming agent may be a defined agent such as a large molecule or a small molecule. A large molecule be a biological molecule such as a protein. DNA, or RNA (such as an mRNA, an miRNA, or a siRNA). The protein may be a small molecule. In some variations, the priming agent may be a small molecule from the group selected from the group consisting of a Nrf2 activator, a SIRT1 (silencing information regulator factor 1) activator, all-trans retinoic acid (ATRA), ML228, MDL 800, Isoquercetin, Fucoidan, Luteolin, Quercetin, 5-aminoimidazole-4-carboxamide riboside (AICAR), 5-Phenylalkoxypsoralen (Psora-4), Thienopyridone (A-769662), Metfonnin, Rapamycin, 5-azacytidine (5-Aza), UM171, SB203580, Fisetin, Atorvastatin, Valproic acid. Sphingosine-1-phosphate (SIP), Astaxanthin (ATX), Succinate, and combinations thereof.

In some variations, the Nrf2 activator may be selected from the group consisting of Dimethyl fumarate (DMF) optionally having a concentration in the range of 10-250 μM, 4 Octyl itaconate (4-OI) optionally having a concentration in the range of 10-500 μM, and Imidazole derivative of 2-cyano-3, 12-dioxooleana-1, 9 (11)-dien-28-oic acid (CDDO-Im) optionally having a concentration in the range of 0.1-10 μM. Curcumin optionally having a concentration in the range of 1-20 μM, and Berberine optionally having a concentration in the range of 0.1-100 μM.

In some variations, the SIRT1 activator may be selected from the group consisting of SRT-2104 optionally having a concentration in the range of 0.01 nM to 10 nM, trans-Resveratrol optionally having a concentration in the range of 0.1 μM-10 μM, trans-Resveratrol optionally having a concentration in the range of 10-200 μM. SRT-1720 having a concentration in the range of 0.1-10 μM, Nicotinamide adenine dinucleotide (NAD) optionally having a concentration in the range of 50-200 μM. Nicotinamide mononucleotide (NMN) optionally having a concentration in the range of 0.08-2.25 μM, or Nicotinamide riboside (NR) having a concentration in the range of 1-10000 μM, 1-100 μM, 100-10000 μM, 10-100 μM, or 100-1000 μM,

In some variations, the at least one defined priming agent may comprise all-trans Retinoic Acid (ATRA) optionally having a concentration in the range of 0.1-500 μM, ML228 optionally having a concentration in the range of 1-10 μM, MDL 800 optionally having a concentration in the range of 5-500 μM, Isoquercetin optionally having a concentration in the range of 0.01-5000 μM, Fucoidan optionally having a concentration in the range of 0.00001-0.001 μM, Luteolin optionally having a concentration in the range of 10-100 μM, Quercetin optionally having a concentration in the range of 0.1-10 μM, 5-aminoimidazole-4-carboxamide riboside (AICAR) optionally having a concentration in the range of 1000-10000 μM. 5-Phenylalkoxypsoralen (Psora-4) optionally having a concentration in the range of 0.01-200 μM, Thienopyridone (A-769662) optionally having a concentration in the range of 10-200 μM, Metformin optionally having a concentration in the range of 1-10000 μM, Rapamycin optionally having a concentration in the range of 0.001-0.1 μM, 5-azacytidine (5-Aza) optionally having a concentration in the range of 0.1-1 μM, UM171 optionally having a concentration in the range of 0.01-0.1 μM, SB203580 optionally having a concentration in the range of 1-10 μM. Fisetin optionally having a concentration in the range of 1-50 μM. Atorvastatin optionally having a concentration in the range of 0.1-20 μM. Valproic acid optionally having a concentration in the range of 500-5000 μM. Sphingosine-1-phosphate (S1P) optionally having a concentration in the range of 0.01-0.1 μM, Astaxanthin (ATX) optionally having a concentration in the range of 0.01-100 μM. Succinate optionally having a concentration in the range of 10-500 μM, or combinations thereof.

In some variations, the at least one priming agent may comprise a conditioned media derived from a population of cells, optionally stem cells, different from the population of MSCs being primed with the at least one priming agent. In some variations, the conditioned media is selected from the group consisting of a corneal stromal stem cell derived-conditioned medium and a limbal epithelial stem cell derived-conditioned medium. In some variations, the volume percentage (concentration) of the conditioned media added in the culture medium to serve as a priming agent is in the range of 5-50%, 10-50%, 15-40%, about 10%, about 15%, about 20%, about 25%, or about 30% with respect to the culture media in which the MSCs are growing.

In some variations, a time period for exposure of the MSCs to the at least one priming agent may be in the range of 12-72 hours, 24-72 hours, or 24-48 hours, or about 24 hours or about 48 hours. In some variations, a time period for exposure of the MSCs to the at least one priming agent may be from seeding until between about 60% and about 90% confluency, between about 70% and 90% confluency, between about 70% and 80% confluency, about 60% confluency, about 70% confluency, or about 80% confluency.

### Combinatorial Priming

In some variations, the MSCs may be exposed to two or more priming agents simultaneously, or serially with partial or no overlap. In some variations, a time period for priming for each or both of the priming agents may be in the range of 12-72 hours. In some variations, the MSCs may be exposed to a first priming agent comprised in a first culture medium from seeding until between about 60% and about 90% confluency, between about 70% and 90% confluency, about 60% confluency, about 70% confluency, or about 80% confluency, then exchanged with a second culture medium comprising a second priming agent and optionally exposed to the second priming agent in the range of 12-72 hours, 24-72 hours, or 24-48 hours.

In some variations, the population of MSCs may be primed at least one Nrf2 activator and at least one SIRT1 activator. In some variations, the MSCs may be primed with at least one Nrt2 activator, and a CSSC-CM. In some variations, the MSCs may be primed with at least one Nrf2 activator, at least one SIRT1 activator, and ATRA.

In some variations, the population of MSCs may be primed with combination of at least one Nrf2 activator and at least one SIRT1 activator. The Nrf2 activator may be selected from the group consisting of Dimethyl fumarate (DMF) optionally having a concentration in the range of 10-250 μM, 4 Octyl itaconate (4-OI) optionally having a concentration in the range of 10-500 μM, or Imidazole derivative of 2-cyano-3, 12-dioxooleana-1, 9 (11)-dien-28-oic acid (CDDO-Im) optionally having a concentration in the range of 0.1-10 μM. The at least one SIRT1 activator may be selected from the group consisting of SRT-2104 optionally having a concentration in the range of 0.01-10 nM, trans-Resveratrol optionally having a concentration in the range of 0.1-10 μM, or SRT-1720 optionally having a concentration in the range of 0.1-10 μM. In some variations, the Nrf2 activator and the SIRT1 activator may be administered to the MSCs simultaneously or serially with partial or no overlap, with the a time period for priming for each or both of the defined priming agents being in the range of 12-72 hours.

In some variations, the population of MSCs may be primed with combination of at least one Nrf2 activator and a CSSC-CM. Without being bound by theory, exosomes secreted by MSC primed with an Nrf2 activator and a CSSC-CM may advantageously be relatively enriched anti-inflammatory factors while having relatively reduced angiogenesis factors compared to exosomes from a population of comparable naïve MSCs. As such, exosomes from MSCs combinatorically primed with an Nrf2 activator and a CSSC-CM may be useful for treating or inducing regeneration in nonvascular tissue such as cornea. The Nrf2 activator may be selected from the group consisting of Dimethyl fumarate (DMF) having a concentration in the range of 10-250 p M, 4 Octyl itaconate (4-OI) having a concentration in the range of 10-500 μM, or Imidazole derivative of 2-cyano-3, 12-dioxooleana-1, 9 (11)-dien-28-oic acid (CDDO-Im) having a concentration in the range of 0.1-10 μM. In some variations, the volume percentage of the CSCC-CM added in the culture medium to serve as a priming agent may be in the range of 5-50%, 10-50%, 15-40%, about 10%, about 15%, about 20%, about 25%, or about 30% with respect to the culture media in which the MSCs are growing. In some variations, the priming of the MSCs may be performed under hypoxic conditions optionally having oxygen in the range of 0.2-10%. In some variations, the population of MSCs may be grown in a first culture medium comprising the CSSC-CM at a concentration of about 20% from seeding until between about 60% and about 90% confluency, then exchanged with a second culture medium comprising DMF at a concentration of about 50 μM and about 100 μM and grown in the second culture medium for a time period in the range of 24-28 hours.

### Culturing Methods

In some variations, the MSCs may be cultured in a 3D bioreactor system in a method selected from the group consisting of hollow-fiber based method, microcarrier-based method and a spheroid-based method.

In some variations, the hollow fiber-based method may comprise (i) providing or obtaining a hollow-fiber bioreactor system; (ii) culturing the stem cells as obtained in step (a) in a xeno-free media to obtain a suspension of cells; (iii) injecting the suspension of cells into a cartridge of hollow hollow-fiber bioreactor system; (iv) incubating the suspension of stem cells for a period in the range of 21-35 days to obtain a population of expanded stem cells; (v) adding a protease, optionally trypsin EDTA, to an extra capillary space of the hollow hollow-fiber bioreactor system comprising the population of expanded stem cells to obtain the expanded stem cell; and (vi) treating the expanded stem cell with a buffer to obtain the expanded stem cells

In some variations, the microcarrier-based method may comprise (i) suspending the microcarriers in a culture medium, to obtain a suspension; (ii) seeding the suspension with the stem cells as obtained in step (a); (iii) culturing the stem cells of step (ii) in a culture medium to obtain a population of expanded stem cells adhered to the microcarriers; and (iv) dissolving the microcarriers of step (iii) by contacting the microcarriers with a dissolution buffer comprising sodium chloride and trisodium citrate, to obtain the expanded stem cells.

In some variations, the spheroid-based method may comprise: (i) pelleting the stem cells obtained in step (a), to obtain a stem cell pellet: (ii) resuspending the stem cell pellet in a culture medium comprising basal medium, to obtain a stem cell suspension; (iii) providing or obtaining stem cell spheroids from the stem cell suspension obtained in step (ii), wherein the stem cell spheroids are having a density of stem cells in a range of 600-10,000 cells per spheroid; and (iv) culturing the stem cell spheroids of step (iii) in a culture medium comprising MSC basal medium to obtain the expanded stem cells.

Exosome purification methods

Exosomes comprised in the primed MSC-derived condition medium may be purified with an exosome purification process.

In some variations, the exosome purification process may comprise: (i) centrifuging the primed conditioned medium at a speed in the range of 90,000-120,000×g for a time period in the range of 70-110 min at a temperature in the range of 2-6° C., to obtain a pellet; (ii) dissolving the pellet in a low serum xeno-free media to obtain crude exosomes; and (iii) performing density gradient ultracentrifugation with the crude exosomes to obtain a fraction of exosomes; and (c) purifying the fraction of exosomes by size exclusion chromatography, to obtain a population of enriched exosomes.

In some variations, the exosome purification process may comprise: (i) subjecting the primed conditioned medium or the conditioned medium to a first centrifugation at a speed in the range of 200-400×g for a time period in the range of 5-20 min. and followed by a second centrifugation at speed in the range of 2000-4000×g for a time period in the range of 10-40 min to obtain a supernatant; (ii) performing centrifugation with the supernatant at speed in the range of 200-400×g for a time period in the range of 5-20 min followed by filtering the supernatant to obtain a secretome; (c) centrifuging the secretome, to obtain a pellet: (d) dissolving the pellet in a low serum xenofree media, to obtain a crude solution; (e) performing density gradient ultracentrifugation with the crude solution, to obtain a fraction comprising exosomes; and (f) purifying the fraction comprising the exosomes by size exclusion chromatography, to obtain a population of enriched exosomes.

### Molecular Characterization of the Primed Exosomes

The priming of MSCs induce characteristic changes in the expression level of certain exosomal proteins, as measured for example by ELISA of enriched exosomes.

In some variations, the combinatorial priming of BM-MSC with CSSC-CM and Nrf2 activator may result in the production of exosomes that are characterized by having as compared to unprimed MSC-derived exosomes one or a combination of two or more of: a lower exosomal expression level of vascular endothelial growth factor (VEGF) and a higher expression level of nerve growth factor (NGF), a higher expression level of hepatic growth factor (HGF), and a higher expression level of sFLT1. In some variations, the higher expression level of HGF may be an at least 1.5 times, at least 1.7 times, at least 2 times, or about 2.2 times higher expression. In some variations, the higher expression level of NGF may be an at least 2 times, at least 2.2 times, at least 2.5 times, at least 3 times, or about 3.2 times higher expression. In some variations, the higher expression level of sFLT1 may be an at least 1.5 times, at least 1.7 times, at least 2 times, or about 2.2 times higher expression. In some variations, the lower expression level of VEGF may be half or ness, a third or less, or a quarter or less expression.

In some variations, the priming of BM-MSC with Nrf2 activator (such as DMF or 4-OI) may result in the production of exosomes that are characterized by having as compared to unprimed MSC-derived exosomes a higher expression level of HGF and/or a higher expression level of NGF. In some variations, the higher expression level of NGF may be an at least 1.5 times, at least 1.7 times, at least 2 times, or about 2.2 times higher expression. In some variations, the higher expression level of HGF may be an at least 1.1 times, at least 1.2 times, at least about 1.3 times, or about 1.4 times higher expression.

### Compositions Based on the Above Methods

In an aspect of the present disclosure, there is provided a population of primed MSCs obtained by the method as described herein.

In an aspect of the present disclosure, there is provided a primed conditioned medium obtained by the method as described herein.

In an aspect of the present disclosure, there is provided a population of primed exosomes purified from the conditioned medium obtained by the method as described herein.

In an aspect of the present disclosure, there is provided a composition comprising the purified primed exosomes as described herein. In some variations, the composition may be formation for parenteral administration. In some variations, composition may be in an ophthalmic composition or an eye drop liquid formulated for application to a corneal surface. In some variations, the eye drop liquid or ophthalmic composition may comprise a biocompatible polymer. In some variations, the ophthalmic composition maybe a hydrogel in which at least a portion of the biocompatible polymers are cross-linked. In some variations, the biocompatible polymer may comprise one or a combination of two or more polymers selected from collagen, a hyaluronic acid, a cellulose, a polyethylene glycol, a polyvinyl alcohol, a poly(N-isopopylacrylamide), a silk, a gelatin, and an alginate. In some variations, one or more of the biocompatible polymers may be modified to be cross-linkable through, for example, thiolation or methacrylation.

In an aspect of the present disclosure, there is provided a composition comprising the primed MSCs as described herein.

In an aspect of the present disclosure, there is provided a composition comprising at least two components selected from the group consisting of: (a) primed stem cells as described herein; (b) the primed conditioned medium as described herein; and (c) the enriched exosome as described herein.

### Therapeutic Methods or Compositions for Use Treating a Condition

In certain embodiments of the present disclosure, there is provided a method for treating a condition in a subject, said method comprising: (a) providing or obtaining the enriched exosomes as described herein; and (b) administering the exosomes to a subject for treating the condition. In some variations, the administration may be on a corneal surface. In some variations, the conreal surface administration may be with an eye drop formulation, which may comprise a biocompatible polymer. The biocompatible polymer may be a crosslinkable polymer, so that the liquid becomes a hydrogel upon crosslinking. In some variations, the administration may be a parenteral or intravtraveous administration. The intravtraveous administration may be through a hepatic portal vein.

In certain embodiments of the present disclosure, there is provided a method for treating a condition in a subject, said method comprising, said method comprising: (a) providing or obtaining the primed conditioned medium as described herein; and (b) administering a therapeutically effective amount of the conditioned medium to a subject for treating the condition.

In certain embodiments of the present disclosure, there is provided a method for treating a condition in a subject, said method comprising: (a) providing or obtaining the primed stem cells as described herein, and (b) administering a therapeutically effective amount of the expanded primed mesenchymal stem cell population to a subject for treating the condition.

In certain embodiments of the present disclosure, there is provided a method for treating a condition in a subject, said method comprising: (a) providing or obtaining a composition as described herein, e.g. enriched exosomes, conditioned cell culture medium, a primed mesenchymal stem cell population; and (b) administering a therapeutically effective amount of the composition to a subject for treating the condition.

In certain variations, the above conditions may be selected from the group consisting of rheumatoid arthritis, systemic juvenile idiopathic arthritis, idiopathic pulmonary fibrosis, acute respiratory distress syndrome (ARDS), acute lung injury (ALI), pneumonia, bronchitis, chronic obstructive pulmonary disease (COPD), COVID-19, coronavirus class of infection, cystic fibrosis, hantavirus, influenza, tuberculosis, systemic lupus, myocardial infarction, osteoarthritis, non-alcoholic fatty liver disease (NAFLD), non-alcoholic steatohepatitis (NASH), liver fibrosis, Mooren's ulcer, neurotrophic ulcer, and corneal keratitis (CK), dry eye disease ulcer, herpetic simplex keratitis, post-LASIK ectasia, postoperative corneal melts, post keratoprosthesis melts, corneal perforations, neurotrophic keratitis (NK), keratoconus Sjögren's syndrome, mucous membrane pemphigoid, Stevens-Johnson syndrome, chemical burns, and thermal burns.

In certain embodiments of the present disclosure, there is provided a composition comprising enriched exosomes, conditioned cell culture medium, or a primed mesenchymal stem cell population) as described herein for use in treating a condition selected from the group consisting of rheumatoid arthritis, systemic juvenile idiopathic arthritis, idiopathic pulmonary fibrosis, acute respiratory distress syndrome (ARDS), acute lung injury (ALI), pneumonia, bronchitis, chronic obstructive pulmonary disease (COPD), COVID-19, coronavirus class of infection, cystic fibrosis, hantavirus, influenza, tuberculosis, systemic lupus, myocardial infarction, osteoarthritis, non-alcoholic fatty liver disease (NAFLD), non-alcoholic steatohepatitis (NASH), liver fibrosis. Mooren's ulcer, neurotrophic ulcer, and corneal keratitis (CK), dry eye disease ulcer, herpetic simplex keratitis, post-LASIK ectasia, postoperative corneal melts, post keratoprosthesis melts, corneal perforations, neurotrophic keratitis (NK), keratoconus Sjögren's syndrome, mucous membrane pemphigoid, Stevens-Johnson syndrome, chemical burns, and thermal burns.

In certain embodiments of the present disclosure, there is provided primed stem cells as described herein or the primed conditioned medium as described herein, or the enriched exosomes as described herein, for use in treating a condition selected from the group consisting of rheumatoid arthritis, systemic juvenile idiopathic arthritis, idiopathic pulmonary fibrosis, acute respiratory distress syndrome (ARDS), acute lung injury (ALI), pneumonia, bronchitis, chronic obstructive pulmonary disease (COPD). COVID-19, coronavirus class of infection, cystic fibrosis, hantavirus, influenza, tuberculosis, systemic lupus, myocardial infarction, osteoarthritis, non-alcoholic fatty liver disease (NAFLD), non-alcoholic steatohepatitis (NASH), liver fibrosis. Mooren's ulcer, neurotrophic ulcer, and corneal keratitis (CK), dry eye disease ulcer, herpetic simplex keratitis, post-LASIK ectasia, postoperative corneal melts, post keratoprosthesis melts, corneal perforations, neurotrophic keratitis (NK), keratoconus Sjögren's syndrome, mucous membrane pemphigoid, Stevens-Johnson syndrome, chemical burns, and thermal burns.

Although the subject matter has been described in considerable detail with reference to certain examples and implementations thereof, other implementations are possible.

### EXAMPLES

The disclosure will now be illustrated with working examples, which is intended to illustrate the working of disclosure and not intended to take restrictively to imply any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein. It is to be understood that this disclosure is not limited to particular methods, and experimental conditions described, as such methods and conditions may apply.

### MATERIALS AND METHODS

### Source of Stem Cells

For the purpose of the present disclosure, mesenchymal stem cells (MSCs) derived from the sources such as human hone marrow (BM), corneal limbal stem cells, umbilical cord (UC), unrestricted somatic stem cells. Wharton's jelly (WJ), dental pulp (DP) and adipose tissue (AD), induced pluripotent stem cells (iPSCs), engineered cells, corneal stromal stem cell-derived (CSSC)-derived conditioned media primed MSCs can be used in the methods and cell-derived products as described herein. The engineered cells mentioned in this context are the cells immortalized with hTERT. The choice of the stem cell type would be target indicated and tissue specific.

### Source of Immortalized Adult Stem Cell Lines (Non-Viral Immortalized MSC Cell Lines):

(1) hTERT immortalized human bone marrow mesenchymal stem cells (hBM-MSCs): Primary BM-MSCs (Naïve) with clinically approved CD105⁺, CD90⁺, CD73⁺ markers were used for naïve exosomes production and CSSCs conditioned media primed naïve BM-MSCs were used for prime variant exosome production for avascular tissue regeneration. Clinically approved non-viral human telomerase enzyme reverse transcriptase (hTERT) induced immortalized BM-MSCs were used for constant production of the exosomes.

(2) hTERT immortalized human Wharton's jelly-derived MSC (WJMSC)/Umbilical cord-derived MSC (UC-MSC) cell line: UC-MSCs with clinically approved CD166⁺, CD90⁺, CD73⁺-markers and CD34⁺, α-SMA⁻ markers were selected and grown in xeno-free media and naïve exosomes were produced from the above cell population expressing the aforementioned markers. Clinically approved non-viral hTERT induced immortalized UCMSCs were used for constant production of the exosomes

### Example 1

OBTAINING AND CULTURING CORNEAL STROMAL STEM CELLS (CSSCs) and NAIVE CELLS (hBM-MSCs. UC-MSCs AND WJ-MSCs)

The present example describes the process of obtaining or culturing stem cells, such as, corneal stromal stem cells (CSSCs), hBM-MSCs, umbilical cord UC-MSCs, and WJ-MSCs. and enriching the stem cells to obtain a population of expanded stem cells under xeno-free culture conditions. The present examples also describe the process of obtaining the conditioned medium from the stem cells as mentioned above.

1.1 Culturing CSSCs under xeno-free conditions, and collection of conditioned media from CSSCs
1.1.1. Isolating and culturing CSSCs under xeno-free conditions

Corneal tissue preserved in corneal storage medium with the validity of 4-5 days was procured. Tissues with the following details on their ‘Tissue Specification Sheet’ were used for the cell extraction and culturing: (1) Tissue that has Date of Expiry for transplantation/cell harvesting; (2) Primary cause of death. Absence of HIV (Human Immunodeficiency Virus), HCV (Hepatitis C Virus). HbsAg (Hepatitis B surface antigen) and Syphilis; (3) Cell count per square mm; (4) Absence of sepsis and scars, any systemic infections, any ocular history that does not render the tissue suitable for cell harvesting.

After thorough screening, human donor-derived corneas were used to derive CSSCs using the protocol under xeno-free conditions. The process for culturing the CSSCs under xeno-free conditions is described below:

Human donor-derived corneas were washed with antibiotic fortified buffered saline (PBS) before extracting limbus which contains CSSCs. In aseptic conditions, a 360° limbal ring was excised using surgical instruments and washed with buffered saline and minced into smaller fragments. The minced tissue fragments were collected into incomplete media (MEM media) and subjected to liberase digestion by adding 20 μL of reconstituted Liberase (Roche) at a concentration of 0.5U to the tissue suspension.

After 16 h of incubation, enzymatic digestion was stopped by adding 2 mL of complete media fortified with 2% human platelet lysate (HPL).

The digested tissues were spun down at 200×g for 3 min at room temperature, in saline supplemented with penicillin and streptomycin followed by various level of passaging.

Passage 0 (P0): During passage 0, the digested explants were resuspended in 5 mL xenofree complete media (MEM+2% HPL, 1X ITS, 10 ng/mL EGF) and cultured in a T25 Corning CelIBIND flask for 14 days. The media was changed every 3 days.

Passage 1 (P1): During passage 1, the cells isolated from the explants were trypsinized with Tryple (1X, Gibco), and resuspended in fresh complete media at the end of 14 days. The cells were seeded at 8000 cells/cm² in T75 CellBIND flasks in passages 1. If 0.7×10⁶ cells were seeded in three T-75 flasks at P1, for next passage (Passage 2; P2) the cells were then split into two T-75 flasks and subsequently, further passaging (Passage 3; P3) were split into four T-75 flasks as per the cell doubling.

Complete media change for P1, P2 and P3, was performed at the following time points:
P1—Day 3: 50% media was replenished, i.e., 2.5 mL of media.
   Day 5: 5 mL media was replaced with 5 mL fresh media.
P2—Day 3: 50% media was replenished. i.e., 5 mL.
   Day 5: 10 mL media was replaced with 10 mL fresh media.
   Day 7: 10 mL media was replaced with 10 mL fresh media.
P3—Day 3: 50% media was replenished. i.e., 5 mL.
   Day 5: 10 mL media was replaced with 10 mL fresh media.
   Day 7: 10 mL media was replaced with 10 mL fresh media.

For P2 and P3, approximately 1.2×10⁶ CSSCs were revived, and cell seeding was done at approximately 8000 cells/cm² density.

Table 1 shows the volume of CSSC spent media collected at Passage 1 (P1), Passage 2 (P2), and Passage 3 (P3).

TABLE 1

| | Volume collection | No. of | Total |
|---|---|---|---|
| Passage No. | per flask (mL) | flasks | volume (mL) |
| P1 | 5 mL/T25 flask | 3 | 20-25 |
| P2 | 25 mL/T75 flask | 2 | 50 |
| P3 | 25 mL/T75 flask | 4 | 100 |

For the quality control, cells at P1, P2 and P3 were characterized (Immunofluorescence imaging) using markers, such as, stem cell markers like CD90, CD73, and CD105, corneal cell specific marker like PAX6 and negative markers like SMA, CD34.

1.1.2. Collection of conditioned media (CSSC-CM) from CSSC culture

From passage 1-2-3 as described above, every media change was accompanied by collection of the spent/conditioned media from the flasks. The spent media was further pre-processed by centrifuging the media at 300×g for 10 min to collect the supernatant. The supernatant was further centrifuged at 3000×g for 20 min at 4° C. followed by re-centrifuging the supernatant at 13000×g for 30 min at 4° C., to collect the further processed supernatant. The media was double-filtered through a 0.45 micron filter and further using 0.22 micron filter to collect the supernatant.

The collected supernatant (conditioned media) was stored at 4° C. for short term (1-2 days) or −80° C. for long term.

1.2. Culturing of naïve hBM-MSCs and collection of conditioned media from naïve hBM-MSCs
1.2.1. Culturing of naïve hBM-MSCs under xeno-free conditions

In order to culture the naïve hBM-MSCs, 1M human BM-MSC (hBM-MSC) cells (passage 2) were procured from the manufacturer with its recommended media (hBM-MSC High Performance Media Kit XF), and hBM-MSC cells were cultured according to the manufacturer's protocol. In brief, booster MSC-Xeno-free 10 mL and basal MSC were thawed at room temperature and transferred aseptically in biosafety cabinet to reconstitute into 500 mL media.

For the purpose of culturing, the vial-hBM-1M-XF was taken from liquid nitrogen (LN) box and thawed in a 37° C. water bath for 2-3 min. The cell vial was aseptically transferred to a 50/15 ml, centrifuge tube where 4 mL culture media was added to the cells drop wise. The cell pellet obtained after centrifugation at 200×g for 10 min was dissolved in 5 mL of complete media and as a quality control, cell count was recorded. The volume of culture media in the tube was made up to 30 mL (recommended by manufacturer's protocol) and cells were seeded into CELLBIND T225 cm² flasks at a density of 2000-3000 cells/cm² and media volume was brought up to 40-45 mL in each flask and incubated at 37° C. and 5% CO₂.

To determine the percentage of confluence cells, cells were observed every day from day 3 onwards. Media was not changed until cells reached up to 80% confluence i.e. (43000-50000) cells/cm², after which the cells were ready to be harvested. During harvesting, the cells were transferred into a biosafety cabinet and spent media was removed, keeping 10 mL of spent media in sterile tubes (15-50 mL) for quenching the trypsin enzyme. Media was removed and the cells were washed with 1×PBS followed by addition of 10 mL TrypLE and incubated in a 37° C. incubator. Cells were checked every 5 min for their detachment from the surface. To stop the TrypLE activity, equal volume of quench (fresh media) or spent media was added to the cells.

The cell suspension was transferred into a sterile 50 mL centrifuge tube and centrifugation was done at 200 g for 10 min. The supernatant was discarded, the cells were resuspended with 4-5 mL of fresh media, and the total volume of cell suspension was measured. After obtaining the suspension, 0.1 mL of cell suspension was transferred into microcentrifuge tubes for cell count and a dilution was performed using Dulbecco's phosphate-buffered saline (DPBS) to get a count range of (0.1-1)×10 cells/mL, and the cells were cryopreserved until needed.

1.2.2 Collection of Conditioned Media from Naïve hBM-MSCs.

As described above, 1M hBM-MSC cell vials were revived and observed from day 3 onwards. Images were captured in phase contrast microscope on day 3, day 4/5 and day 6/7; with the cell density as displayed in table 2. Cells were washed on day 4/5 depending on the cell density (43000-50000 cells/cm²), either 1-2× with 20 mL of PBS, and the media was changed to Rooster EV collect medium. After 48 hours of incubation, the conditioned media was collected, and the cells were harvested following the protocol as described in example 1.2.1 above. Subsequently, the cells were counted using cell counter. The collected conditioned media was immediately processed with the pre-processing steps as described in example 1.1.2 above and the pre-processed conditioned media was then stored at 4° C. (for short term storage up to 24 hours) or at −80° C. (for long term storage up to 1 month).

Table 2 shows cell density (cells/cm²) on day 3, day 4, and day 6/7.

TABLE 2

| | Cells/cm2 (counting | Final cell number/ |
|---|---|---|
| Day of Culture | by Image J) | T225 flask |
| Day 0, seeding density | (2000-3000) | (0.45-6.0) × 10⁶ |
| Day 3 (72 h of seeding) | (13000-15000) | (2.0-3.3) × 10⁶ |
| Day 4/5 | (43000-50000) | (9-12) × 10⁶ |
| Day 6/7 (after 48 h of EV | (43000-50000) | (9-12) × 10⁶ |
| collect treatment) | | |

1.3. Culturing of Naïve UCMSCs and Collection of Conditioned Media from Naïve UCMSCs

### 1.3.1. Culturing of Naïve UCMSCs

UC-MSCs cells were procured from the manufacturer and cultured in recommended xeno-free media, as per the manufacturer's protocol. In brief. MSC-XF and basal-MSCs were thawed at room temperature in the dark to reconstitute one bottle of 500 mL media. Further, vial-human umbilical cord-1X-XF (Xeno-free) (hUC-1M-XF) obtained after passage was thawed in a water bath at 37° C. and the cells were transferred aseptically in Biosafety Cabinet. Cells were transferred into 15/50 mL centrifuge tubes in 10 mL media volume and centrifuged at 280×g for 6 min. The supernatant was discarded, and the cell pellet was dissolved in 20 mL of media. The cell suspension was further divided in four T75 flasks and two T225 flasks, where the seeding density for T75 flask was kept within the range of 2000-3000 cells/cm² and for T225, the seeding density was within the range of 2000-3000 cells/cm².

For the T225 flasks and the T75 flasks, 45 mL and 15 mL of media volume was used respectively, and the media was maintained at 37° C. To determine the percentage of confluence, the cells were observed microscopically from day 3 onwards and images were captured followed by cell counting using the Image J software.

Cells were further observed on day 4 and 5 when the culture was confluent to 80%, (e.g., cells were at a cell density of 60k-100k cells/cm² for a T225 flask). The cells were harvested by transferring the flask into the biosafety cabinet and collecting the spent media in sterile container (˜10 mL) for quenching the trypsin enzyme. After removing the media, 10 mL or 3 mL of TrypLE was added to each T225 or T75 flask respectively, and the flasks were incubated in the 37° C. incubator. The cell culture was checked every 5 min until the cells were detached from the surface or the cells were dislodged by gentle tapping. To stop the TrypLE activity, equal volume of the quench or the spent media were added to the cell suspension and transferred into a 15/50 ml, centrifuge tube for centrifugation at 280×g for 6 min. After the supernatant was discarded, the cells were resuspended in 4-5 mL, of fresh media and the total volume of cell suspension was measured. Cell counting was performed using 0.1 mL of cell suspension after a dilution was done with DPBS/media to get a count range of (0.1-1)×10⁶ cells/mL and the cells were cryopreserved until further use.

1.3.2. Collection of Conditioned Media from Naïve UCMSCs

To produce extracellular vesicle (EV) from naïve UC-MSCs, 1M cell vials were revived as described above and cells were observed from day 3 onwards to capture the images using contrast microscope on day 3, day 4/5 and day 6/7 with the cell density as provided in table 3. On day 4/5, depending on the cell density (60-100k) cells/cm² (T225 flask) i.e. nearly at 80% confluence, the cells were washed twice with 20 mL of PBS and the media was changed to extracellular vesicle (EV) collect medium. After 48 hours of incubation, the conditioned media was collected, and the cells were harvested and counted with the cell counter. The collected conditioned media was immediately processed with the pre-processing steps as described in example 1.1.2 above.

TABLE 3
Cell density (cells/cm²) on Day 3, Day 4 and day 6/7 (Passage 4 expansions)

| | Cells/cm² in T225 | Final cell | Cells/cm² in T75 | Final cell |
|---|---|---|---|---|
| | flask (counting | number/T225 | flask (counting | number/T75 |
| Day of Culture | by Image J) | flask | by Image J) | flask |
| Day 0, seeding density | 2000-3000 | 0.45-6.0 × 10⁶ | 3300 | 2-3 × 10⁵ |
| Day 4/5 | 60-100K | ≤(13.5-22.5) × 10⁶ | 50-60K | 3-5 × 10⁶ |
| Day 6/7 (after 48 h of | 60-100K | ≤(13.5-22.5) × 10⁶ | 50-60K | 3-5 × 10⁶ |
| EV collect treatment) | | | | |

### 1.4. Cell Sorting for Selection of Population of Stem Cells, its Subtypes

One of the crucial aspects of the present disclosure is the isolation of unique sub-populations of mesenchymal stem cells (MSCs) from stem cells such as UC-MSCs/WJ-MSCs expressing signature set of markers to produce exosome with desired therapeutic effects, such as anti-inflammatory, anti-fibrosis, pro-wound healing. (pro-/anti-) angiogenesis, and re-innervation properties. This feature is important and different from the conventional methods that are known in the literature, as the conventional methods uses heterogenous population of stem cells, such as UC-MSCs cells, whereas the method of the present disclosure deploys the unique sub-populations of MSCs that offer superior therapeutic activity (anti-inflammatory, anti-fibrosis, pro-wound healing, angiogenesis (pro-/anti-), reinnervation).

The common signature set of markers that are expressed by stem cells, such as UCMSCS and WJMSCs are listed in table 4.

TABLE 4

| S. No. | MARKER | EXPRESSION | ENRICHMENT PROPERTY |
|---|---|---|---|
| 1 | GSTT1 | NEGATIVE | Increased proliferative rates, clonogenic potential, and longer |
| | | | telomeres |
| 2 | CD142 | LOW | Could trigger blood clotting in COVID-19 and other vulnerable |
| | | | patient populations and further promote the risk for |
| | | | thromboembolism. |
| 3 | CD146 | POSITIVE | Expression correlates with telomerase activity and declines with |
| | | | passaging |
| 4 | CD146/CD107a/ | POSITIVE | Enriched expression with enhanced secretory capacity, robust |
| | CXCR4/LepR | | secretome and immunomodulatory function with stimulated target |
| | | | immune cells. |
| 5 | CD105 | POSITIVE/ | CD105- population shows higher immunomodulation capacity |
| | | NEGATIVE | |
| 6 | CD98 | POSITIVE | Adhesion and migration |
| 7 | CD49c | NEGATIVE | Immunosuppressive |
| 8 | CD98 | POSITIVE | Adhesion and migration |
| 9 | Cell size | small | Flow cytometric analysis revealed the expression of certain MSC |
| | | | stem cell markers including CD44, CD73, CD90 and CD105, at |
| | | | higher levels in the small-sized population. Further, this small- |
| | | | sized subpopulation exhibited a higher proliferative capacity. |
| 10 | CD141 | POSITIVE | — |
| 11 | CD143 | NEGATIVE | Adult stem cell marker |
| 12 | IL-6 | low/high | Associated with MSC pluripotency and immune privileges |
| | | (indication | |
| | | dependent) | |
| 13 | Oct-4/HLA-G | POSITIVE/ | These cells are clonogenic, retain long telomeres, and maintain a |
| | | POSITIVE | stable phenotype and a normal karyotype throughout passages. |
| 14 | CD46 | POSITIVE | Protection from complement-mediated lysis |
| 15 | CD55 | POSITIVE | protection from complement-mediated lysis |
| 16 | CD152 | POSITIVE | Immunosuppressive |
| 17 | CD153 | POSITIVE | Immunomodulatory |
| 18 | Fas ligand | POSITIVE | induces apoptosis in FAS expressing cells (cancers) |
| 19 | CD205 | POSITIVE | Expression declines with passaging, indicating unreported roles |
| 20 | CD106 | POSITIVE | in maintaining stemness, inhibiting senescence |
| 21 | CD24 | POSITIVE | |
| 21 | CD54 | POSITIVE | |
| 22 | CD59 | POSITIVE | |
| 23 | CD243/P- | POSITIVE | |
| | glycoprotein | | |
| 24 | CDKN2A p16 | NEGATIVE | Upregulated with passaging, induction of senescence |
| 25 | CD81 | POSITIVE | — |
| 26 | CD47 | POSITIVE | Immune evasion |
| 27 | CD276 | POSITIVE | — |
| 28 | CD151 | POSITIVE | — |
| 29 | CD147 | POSITIVE | — |
| 30 | CD13 | NEGATIVE | — |
| 31 | Explant | EXPLANT | Explant yields more cells |
| | isolation vs | | |
| | mechanical | | |
| | disruption | | |
| | protocol | | |
| 32 | CCL2/CCL5/IL-8 | LOW | Pro-inflammatory |
| 33 | VEGF/PDGR/ | LOW | Angiogenesis |
| | TGF-b2/b-FGF | | |
| 34 | CD34 | NEGATIVE | Non-UCMSC/hBM-MSC type contamination (differentiated |
| 35 | CD45 | NEGATIVE | cells, immune cells) |
| 36 | CD31 | NEGATIVE | |
| 37 | CD80 | NEGATIVE | |
| 38 | CD86 | NEGATIVE | |
| 39 | CD58 | POSITIVE | |
| 40 | CD273 | POSITIVE | Immunomodulatory (>90% UC-MSC and <35% BM-MSC) |
| 41 | CD54 | POSITIVE | High colony-forming ability and multi-lineage potential |
| 42 | CD119 | POSITIVE | — |
| 43 | CD268 | POSITIVE | — |
| 44 | CD133 | POSITIVE | Transplanted CD133+ human stem cells primarily enhanced |
| | | | hepatic angiogenesis and neovascularization and contributed to |
| | | | repair in a paracrine manner by creating a permissive |
| | | | environment that enabled proliferation and survival of damaged |
| | | | cells rather than by direct differentiation to hepatocytes. A dual |
| | | | advantage of CD133+ cell therapy in hepatic disease is suggested |
| | | | based on its capability of hematopoietic and endothelial |
| | | | differentiation. |
| 45 | CD106 | POSITIVE | Exhibits unique immunoregulatory activity |

Further, apart from the markers listed in the above table, the other cell markers that are expressed at higher levels in MSCs, includes but not limited to CD44, CD73, and CD90.

1.4.1 Isolation of sub-population of UCMSCs

At the end of first passage (P4) or after the expansion of hUC-1M-XF cell vial as described in example 1.3.1, the cells were sorted based on the clinically approved MSCs surface marker CD90, CD73, and CD166 to obtain two sub-population of UC-MSCs using a flow cytometry-based sorting protocol. The two sub-population of UC-MSCs were:

First sub-population: The first sub-population of stem cells that expressed clinically approved MSCs surface markers, such as, CD90⁺, CD73⁺ and CD166⁺ were selected.

Second sub-population: MSCs with CD166⁺, CD90⁺, CD73⁺-positive population were re-sorted to provide two sub-types of first sub-population (enriched therapeutically unique UC-MSCs population):
(i) CD146+, CD54+, CD58+ and CD142+positive population;
(ii) CD146+, CD54+, CD58+ and CD142-(low/negative) population.

The above UC-MSCs sub-types were maintained in xeno-free media with two more passages (P5 and P6) followed by collecting the conditioned media as described in example 1.1.2 of the present disclosure.

1.5. Culturing of hTERT Immortalized WJ-MSCs

### 1.5.1 Cells Revival and Expansion of WJ-MSCs

The culture flasks were pre-coated with animal component-free cell attachment substrate, prior to revival. Briefly, the substrate (1:300, diluted in 1×PBS) was added in the culture flask and incubated for at least 2 h at room temperature. The excess of the substrate solution was removed, and the flask was rinsed twice with PBS (1X). After rinsing, 6 ml of growth medium was added to a 25 cm² culture flask and placed in the incubator for at least 30 min to allow the medium to reach its normal pH. Frozen cell vial was taken from liquid nitrogen and rinsed outside with 70% ethanol and pre-warmed in the hand until one last piece of frozen cells was seen. Thawed vial was transferred to a 15 mL centrifugation tube pre-filled with 9 mL of medium pre-cooled to 4° C.

The cells were further centrifuged at 400×g for 5 min at room temperature and the supernatant was discarded followed by resuspending the cells in 1 mL of pre-warmed medium. The cells were transferred to the prepared culture flask (T25 cm²) and incubated at 37° C. As a quality control (QC), cells were counted and recorded, followed by changing the media after 24 hour and passaging the cells at approximately between 70-80% confluency.

### 1.5.2 Sub Culturing of WJMSCs/hTERT Immortalized WJMSCs

Sub culturing was performed using pre-coated culture flasks with 70-80% confluency with a cell density of 28000 cells/cm². For detachment of cells, the TrypLE Select enzyme solution (20 μL/cm²) was added and the cells were washed twice with PBS (160 μL/cm²). The flask was incubated at 37° C. for approximately 2-3 min and cell detachment was observed under microscope. The growth medium was added to the cells and centrifuged at 400×g for 5 min followed by resuspending in 1 mL of media and coated using Trypan blue. The cells were seeded (7000 cells/cm²) to coated culture vessels supplemented with 240 μL/cm² of growth medium where, a split ratio of 1:4 twice a week was maintained after reaching 80% confluence (e.g., approximately 28000 cells/cm²), and the cells were then trypsinized using TrypLE select enzyme.

Subsequently, the cells were re-suspended in growth medium and centrifuged at 400×g for 5 min, and the cells were suspended in 1 mL of cryopreservation media. CryoStor (CS10) that corresponds to 5×10⁵ cells/mL, 1 mL of the cell suspension was transferred in pre-cooled cryovial and transferred to −80° C. for overnight or to liquid nitrogen for long term storage. For further use, the cells were grown in MesenCult-ACF Plus medium supplemented with MesenCult-ACF Plus 500X Supplement, 200 μg/mL G418.

### Example 2

### Methods of Expanding Stem Cells in 3D Culture

MSCs (naïve/hTERT immortalized) derived from various sources as described in example 1, were cultured in 3D culture-based systems to obtain the expanded stem cells. The different 3D culture-based methods are as follows:
(i) Culture in 3D microcarriers: The culture of MSCs on 3D microcarriers is described in detail in the pending application PCT/IN2020/050622, which is incorporated in its entirety in the present disclosure.
(ii) Culture as 3D spheroids: The culture of MSCs on 3D microcarriers is described in detail in the pending application PCT/IN2020/050622, which is incorporated in its entirety in the present disclosure.
(iii) Culture in hollow fiber bioreactors.
   2.1 2D Culture of hMSCs in CellSTACK Flasks

Human mesenchymal stem cells (hMSCs) were purchased at passages 1-2 and expanded as per manufacturer's protocol to generate a working cell bank. For large scale expansion of hMSCs, i.e., expansion in CellSTACK culture chambers (10-stack), 20 million hBM-MSCs from the working cell bank were seeded in the chambers at a seeding density of 3145 cells/cm² (Corning, cat. #3271). A complete culture medium was prepared as recommended by the manufacturer's protocol and cells were grown for 4 days until cell confluency reached approximately 80-90%.

For cell harvesting from the CellSTACK flasks, the medium was removed, and cells were incubated at 37° C. for 6-8 min after adding 0.25% Trypsin-EDTA. Further, to quench the trypsin activity, 200 μL of 2% MSC-screened FBS, prepared in DPBS (without Ca++. Mg++) was added to the cells, followed by collecting the suspension in 50 mL centrifuge tubes and centrifuged at 200×g for 10 min. The suspension was further resuspended in a final volume of 20 mL complete medium and injected into the hollow-fiber bioreactor system.

2.2 3D Culture of hMSCs in FiberCell Hollow Fiber Bioreactors

The cells were seeded in separate hollow-fiber bioreactors at 90-220×10⁶ cells/cartridge (20-kD MWCO, 4000 cm², polysulfone fiber cartridge) and maintained in xeno-free complete medium, where the hollow-fiber bioreactor system was prepared and used according to the manufacturer's instructions. All pre-inoculation steps were performed using sterile D-PBS−/−. Prior to the injection of the cell suspension, 1 mL of media was drawn from the media reservoir to verify total glucose content using a glucose meter and L (+)-Lactate using the Lactate Assay Kit (50 μL of media diluted 1000×). To inoculate the cells in the bioreactor system, the cell suspension (20 mL) prepared was injected into the cartridge following the manufacturer's procedure. The flow rate of the pulsatile perfusion pump was set at 22 times per minute for the first 2-3 days of the 28-day cell inoculation period.

The media volume in the extracellular capillary space was maintained at 250 mL and circulated at a system flow rate of 25 times per minute from days 3-17 of the 28-day cell inoculation period into the bioreactor. After day 17, the media volume was doubled to 500 mL with the same flow rate. A 1-mL aliquot of the medium from the media reservoir was collected every 2-3 days to monitor the glucose content and pH. At the end of the 25 days culture period, the conditioned medium was retrieved following hMSCs retrieval using 40 mL of Trypsin-EDTA 0, and 25% cells were injected into the extra capillary space and incubated for 10 min at 37° C. The trypsinized cells were pushed through using PBS until 60 mL of cell suspension was obtained. The harvested cell suspension was further quenched with an equivalent volume of 2% MSC-screened FBS prepared in DPBS (without Ca++, Mg++) and centrifuged at 200×g for 10 min and used for cell viability counts using the Trypan Blue exclusion kit, before being processed for downstream analyses.

### Example 3

### Priming of Stem Cells

The present example demonstrates one of the important aspects of the present disclosure, which is the priming of the stem cells derived from various sources as described in example 1. The priming of the stem cells is done in presence of different priming agents, such as small molecules having molecular weight less than 800 Da, and macromolecule having molecular weight more than 800 Da. The priming of stem cells with one or more priming agents (single or combinatorial priming respectively) is done to enhance regenerative, stemness, anti-inflammatory, and anti-fibrotic properties of the MSCs. Naïve MSCs or primed MSCs can be used as such or in addition to naïve exosomes or primed exosomes (obtained by the method as described in the forthcoming examples) for the therapeutic applications.

### 3.1 Priming Agents Used in the Present Disclosure

3.1.1. Priming of Stem Cells with Small Molecules

Regenerative enhanced (licensed to augment in-vitro and in-vivo sternness, viability, engraftment abilities) by priming was performed using various small molecules (hydrophobic agents) including, but not limited to SIRT 1 activator, Nrf2 activator in absence or presence of the physical inducer (hypoxia). As most of these compounds (small molecules) are hydrophobic in nature and hence not water-soluble, they were first dissolved in non-toxic organic solvents, such as dimethyl sulfoxide (DMSO), ethanol, acetone, etc., at high concentrations and subsequently diluted in an aqueous medium (PBS, saline or cell culture medium) to yield working concentrations or formulated in lipid-based carriers, such as liposomes, for treatment of MSCs.

Table 5 lists the priming agents (small molecule) with the working concentration and duration of treatment.

TABLE 5

| | Working | | |
|---|---|---|---|
| | Concentration | Duration | |
| Priming Agent | range (μM) | (hours) | Therapeutic effect |
| Nicotinamide adenine | 50-200 | 24-72 | Enhanced stemness of transplanted |
| dinucleotide (NAD) | | | MSCs, improves anti-inflammatory, |
| Nicotinamide | 0.08-2.25 | 24-72 | anti-fibrotic, pro-wound healing |
| mononucleotide (NMN) | | | properties of MSCs by regulating |
| Nicotinamide riboside | 1-10000 | 24-72 | SIRT family of proteins (SIRT1, 2) |
| (NR) | | | and delays the ageing phenomenon. |
| SRT-1720 | 0.1-10 | 24-72 | |
| SRT-2104 | 0.00001-0.01 | 24-72 | |
| trans-Resveratrol | 0.1-10 | 24-72 | |
| MDL 800 | 5-500 | 12-72 | Activates the SIRT6 enzymatic |
| Isoquercetin | 0.01-5000 | 12-72 | activity that regulates genomic |
| Fucoidan | 0.00001-0.001 | 12-72 | stability, oxidative stress, DNA |
| | | | repair, metabolism and inflammation, |
| | | | cancer and ageing |
| 4-Octyl itaconate | 10-250 | 24-72 | Enhanced stemness of transplanted |
| CDDO-Im | 0.02-0.1 | 24-72 | MSCs, improves anti-inflammatory, |
| Dimethyl fumarate (DMF) | 10-250 | 24-72 | anti-fibrotic properties of MSCs |
| Curcumin | 1-20 | 24-72 | and exosomes |
| Berberine | 0.1-100 | 12-72 | Reduces the reactive oxygen species |
| Luteolin | 10-100 | 72-24 | Attenuates production of the |
| | | | proinflammatory mediators such as |
| | | | NO, iNOS and COX-2 |
| Quercetin | 0.1-10 | 0-144 | Promotes BMSC proliferation and |
| | | | osteogenic diferentiation |
| 5-aminoimidazole-4- | 1000-10000 | 24-72 | Cardiac production and release of |
| carboxamide-1-β-D- | | | FGF21 are induced by catecholamine |
| ribofuranoside | | | and SFAs. |
| (AICAR (AICAR) | | | |
| Thienopyridone | 10-200 | 24-72 | Stimulation of fatty acid oxidation, |
| (A-769662) | | | increased glucose uptake and |
| | | | glycolysis, inhibition of fatty acid |
| | | | synthesis and cholesterol synthesis, |
| | | | and inhibition of hepatic |
| | | | gluconeogenesis. |
| Metformin | 1-10000 | 12-72 | Regulates the immuno modulatory |
| | | | function |
| All-trans retinoic | 0.1-500 | 24-48 | ATRA was found to increase the |
| acid (ATRA) | | | efficacy of cell therapy when MSCs |
| | | | are pre-conditioned with it. |
| Rapamycin | 0.001-0.1 | 12-72 | Protects cells from senescence. |
| ML228 | 1-10 | 24-72 | Acts on the HIF pathway through |
| | | | metal chelation. |
| Succinate | 10-500 | 24-72 | Enhances anti-inflammatory |
| | | | properties of stem cells. |
| 5-Phenylalkoxypsoralen | 0.01-200 | 24-96 | Increases health span of stem cells. |
| (Psora-4) | | | |
| 5-azacytidine (5-Aza) | 0.1-1 | 24-72 | Promotes differentiation capacity of |
| | | | MSCs |
| UM171 | 0.01-0.1 | 24-48 | Promotes stem cell renewal |
| SB203580 | 1-10 | 24-96 | Increase therapeutic activity of MSCs |
| Fisetin | 1-50 | 24-96 | Inhibits senescence |
| Atorvastatin | 0.1-20 | 24-72 | Enhanced homing of MSCs |
| Valproic acid | 500-5000 | 24-72 | Promotes stemness and migration |
| Sphingosine-1- | 0.01-0.1 | 24-72 | capacity of MSCs |
| phosphate (S1P) | | | |
| Astaxanthin (ATX) | 0.01-100 | 24-72 | Antioxidant, improves cardiac |
| | | | function and stem cell potency, anti- |
| | | | fibrotic, induces proliferation of |
| | | | neural progenitor cells, and is a |
| | | | geroneuroprotector |

Silencing information regulator factor 1 (SIRT 1) activator: In an example, the small molecule is silencing information regulator factor 1 (SIRT1) activator. SIRT1 is a NAD-dependent histone deacetylase has an important role in cell metabolism, cell survival and cell senescence, DNA repair, inflammation, cell proliferation and in neurodegenerative diseases (Zhu. Y.g., et al., Human mesenchymal stem cell microvesicles for treatment of Escherichia coli endotoxin.

SIRT1 activator included, but not limited to SRT-2104, SRT-1720, trans-Resveratol.

Nuclear factor crythroid 2-related factor 2 (Nrf2) activator: Nuclear factor crythroid 2-related factor 2 (Nrf2) is ubiquitously expressed in most eukaryotic cells and functions to induce a broad range of cellular defences against exogenous and endogenous stresses, including oxidants, xenobiotics, and excessive nutrient/metabolite supply. The Nrf2 activator acts as a critical regulator of stem cell quiescence, survival, self-renewal, proliferation, senescence, and differentiation.

The Nrf2 activator includes, but not limited to Dimethyl Fumarate (DMF), Imidazole derivative of 2-cyano-3, 12-dioxooleana-1, 9(11)-dien-28-oic acid (CDDO-Im), and 4-octyl itaconate (4-OI). Other activators include families: Arylcyclohexyl pyrazoles, Sulfonyl coumarins, 1, 4-Diaminonaphthalene core containing, Benzenesulfonyl-pyrimidone, 1,2,3,4-Tetrahydroisoquinoline core containing compounds.

Nrf2 inducing peptides (blockers of Nrf2/Kcap interaction) that can be used as priming agents in the present disclosure: LDEETGEFL-NH2, (NH2-RKKRRQRRR-PLFAERLDEETGEFLPNH2), Ac-DPETGEL-OH, Ac-DEETGEF-OH, LQLDEETGEFLPIQGK(MR121)-OH, Ac-LDEETGEFL-NH, AcDPETGEL-NH2, Ac-NPETGEL-OH.

### 3.1.2. Hypoxia Mediated Priming

Hypoxia priming is a mimicking trait for in-vivo MSC niche microenvironment and it has potential to improve regenerative, survival and angiogenesis potential of MSCs. Hypoxia regulates the cell metabolism during expansion of MSCs and provides resistance to oxidative stress and improves engraftment and survivability in ischemic microenvironments. HIF-1α induction has been detected in hypoxic priming, and HIF-1a over expression showed the induction of miR-15, miR-16, mIR-17, miR-31, miR-126, miR-145, miR-221, miR-222, miR-320, miR-424 that are related to angiogenesis capacity of MSCs.

In the present disclosure, hypoxia mediated priming was done in presence of oxygen in the range of 0.2-10%.

### 3.1.3. Light Mediated Priming

Light mediated priming or Photobiomodulation is another inducer platform that involves the use of non-ionizing forms of light sources in the visible and near infrared spectrum. This non-thermal process results in both photophysical and photochemical processes at the biological scale under the influence of endogenous chromophores. The wavelength of the light source involved are of the following ranges (300-650 nm and 800-1400 nm) while the light energy is of 0.5-4 J/cm². Studies have shown that the proliferation of MSCs at low and high density is also influenced by irradiance (5-20 mW/cm²) which could be either single or multiple dose irradiance.

3.1.4. Priming of Stem Cells with Macromolecule

In the present disclosure, the macromolecules were used for priming stem cells, wherein macro molecules are referred to as biological agents, such as proteins, lipids, nucleic acids, growth factors, cytokines, components of conditioned media, etc. For the purpose of the present disclosure, naïve MSCs-derived conditioned medium as described in example 1, was used to prime naïve MSCs derived from different origin.

In the present disclosure, naïve MSCs are being referred to as A and exosomes derived from naïve MSCs (A) as B. Priming of A using small molecules such as nuclear factor erythroid related factor 2 (Nrf2) activator, silencing information regulator factor (SIRT1) activator and macro molecules, is referred to as A′ and exosomes derived from primed MSCs (A′) as B′. The process of single priming with single inducer molecule and combinatorial priming using small molecules or macromolecules are described in the forthcoming examples below.

3.2. Single Priming Protocol of hBM-MSCs with CSSC Conditioned Media

hBM-MSCs, passage 4 cells, were maintained according to the process described in example 1.2. Priming of hBM-MSCs was performed with the media, supplemented with corneal stromal stem cell (CSSC)-derived conditioned media (macromolecule) having a volume percentage in the range of 10-20%. Xenofree culture of CSSCs and collection of conditioned media for priming at a final concentration of 20% was performed during the CSSC maintenance, hBM-MSCs were further expanded until 80-85% confluence (i.e. 43000-50000 cells/cm²) and used for exosome production. Conditioned media collection and processing was performed as described in examples 1.2 and 1.1.2 above.

Table 6: Single priming protocol of hBM-MSCs with CSSC conditioned media.

TABLE 6

| Cells seeding | | Shifting to EV | | |
|---|---|---|---|---|
| density, flasks | | collect at 80-85% | | |
| required for 1M | Day of media change | confluence i.e. | Collection of EV | |
| hBM-MSCs | and priming media | 43000-50000 | collect media | Conditioned |
| expansion (P4) | composition | cells/cm² | after 48 h | media volume |
| 2000-3000 cells/cm², | Day 3and5, complete | Day 5/6, | Day 7/8, | 40-45 L/flask |
| 2-5 T225 cm² flasks | media supplemented | 40-45 mL/flask. | 43000-50000 cell/cm², | |
| | with 20% CSSC | | harvesting density | |
| | conditioned media | | | |

3.3. Single Priming Protocol of hBM-MSCs with Nrf2 Activator

hBM-MSC, Passage 4 cells were maintained according to the process described in example 1.2 above and cells were treated with Nrf2 activators or inducers (DMF, 4-OI). The cells were washed with PBS once they reached 70-80% confluence and replenished with fresh media with 100 μM DMF or 100 μM 4-OI for 24 h prior to switching to EV collect followed by conditioned media collection and processing procedure as described in examples 1.2 and 1.1.2 above.

Table 7: Single priming protocol of hBM-MSCs with Nrf2 activator.

TABLE 7

| Cells Seeding | | 24 h-72 h exposure | | | |
|---|---|---|---|---|---|
| density, flasks | | with Nrf2 activator | | | |
| required for 1M | Day of media | at 70-80% confluency/ | | Collection of EV | |
| hBM-MSCs | change and media | 30000-40000 cells/cm² | Shifting to | collect media | Conditioned |
| expansion (P4) | composition | @ day 4/5) | EV collect | after 48 h | media volume |
| 2000-3000 cells/cm², | No media change | DMF (100 μM), 4 | Day 6/7, | Day 6/7, | 40-45 mL/flask |
| 3-5 T225 cm² flasks | up to reach its | OI (100 μM) with | 40-45 mL/flask. | 43000-50000 cell/cm², | |
| | confluence at 80% | complete media | | harvesting density | |

3.4 Single Priming Protocol of UCMSCs with SIRT1 Activator (SRT2104 or Trans-Resveratol) (EXO VARIANT B′)

Two UC-MSC cell types were cultured separately in presence of SIRT1 activator (SRT-2104 or trans-Resveratrol) with a concentration lesser than the IC50 value of SRT-2104. IC50 value was determined for both the sub populations using a concentration range of 0.04-3.78 nM or 24-1962 ng/mL for SRT-2104 in UC-MSC cells. The working concentration range for RSV was 0.1-2.5 μM or 22.85-571.25 pg/mL for priming in UC-MSCs. UC-MSCs were cultured in presence of SRT-2104 or RSV to a confluence of 80%. Subsequently, EV collect treatment, conditioned media collection was performed for the primed exosomes production. After priming, the UC-MSC primed conditioned media/secretome was screened using the ELISA assay to detect the expression of TNF-α, IFN-γ, IL-10, and HGF for both the cases.

The primed exosome variant was characterized by ELISA detecting the expression level of exosome cargo molecules such as SIRT1, HGF, IDO, IL-10, NRF2, VEGF, and NGF and based on these results, the concentration for SRT2104 or RSV treatment was finalized for UC-MSC priming.

3.5 Single priming protocol of UC-MSCs with Nrf2 activator (DMF or 4-OI OR CDDO-IM)-(EXO VARIANT B′)

UC-MSCs were cultured following the process as described in example 1.3. The cells that were passaged up to 5-6 passaging stage, were used for primed exosome production. (B′). The UC-MSCs were treated with Nrf2 activator (DMF, 4-OI or CDDO-Im) with a range of working concentration such as DMF ((1.44-36 mg/mL) or (10-250 μM)), 4 OI ((0.0024-0.060) mg/mL or (10-250)) μM, CDDO-Im ((2.56-128 μg/mL) or (0.2-1)) μM.

After priming, the UC-MSC primed conditioned media/secretome was screened with ELISA assay kits to detect expression levels of TNF-α, IFN-γ, IL-10, and HGF. The primed exosome variant was characterized by ELISA assays to detect the expression levels of exosome cargo molecules such as Nrf2, IL-10, HGF, and VEGF where the concentration of DMF or 4-OI or CDDO-Im was finalized for UC-MSC priming.

3.6 Combinatorial Priming of hBM-MSCs with CSSC-Derived Conditioned Media AND Nrf2 activator—(EXO VARIANT B′)

Priming of hBM-MSC was performed with the media, supplemented with CSSC-derived conditioned media (volume percentage in the range of 10-20%). Xenofree culture of corneal stromal stem cells (CSSC) and collection of conditioned media for priming at a final concentration of 20% was performed during CSSC maintenance. The hBM-MSC cells were thawed and seeded at 2000-3000 cells/cm² in T225 cm² flasks and media was supplemented with 10-20% CSSC-derived conditioned media. Once, hBM-MSCs reached about 70-80% confluency, the cells were washed in PBS and the cells were then replenished with the Nrf2 activator (for example 100 μM DMF or 100 μM 4-OI) for 24-72 hours prior to switching to extracellular vesicles (EV) collection media.

Table 8 shows the protocol of combinatorial priming of hBM-MSCs with CSSC conditioned media and Nrf2 activator—(Exo variant B′).

TABLE 8

| Cells seeding | | | Shifting to EV | | |
|---|---|---|---|---|---|
| density, flasks | | 24 h exposure with | collect at 70-80% | | |
| required for 1M | | Nrf2 activator at | confluence/ | Collection of EV | |
| hBM-MSCs | Priming media | 70-80% confluence/ | 43000-50000 | collect media | Conditioned |
| expansion (P4) | composition | 30000-40000 cells/cm² | cells/cm² | after 48 h | media volume |
| 2000-3000 cells/cm², | Complete media | DMF (100 μM) or | 40-45 mL/Flask. | 43000-50000 | 40-45 mL/flask. |
| 3-5 T225 cm² flasks | supplemented | 4-OI (100 μM) in | | cells/cm², | |
| | with (10-20) % | supplemented | | harvesting | |
| | CSSC | media. | | density | |
| | conditioned | | | | |
| | media | | | | |

3.7 Combinatorial Priming of UC-MSCs with the SIRT1 Activator and the Nrf2 Activator in Absence of Hypoxia—(EXO VARIANT B′)

UC-MSCs and selected sub-population of UC-MSCs were cultured following the protocol as described in example 1.3 followed by priming of the expanded population of UC-MSCs stem cells and it is sub-population in the presence of SIRT1 activators such as, SRT-2104 (0.00001-0.01 μM) or trans-Resveratol (RSV) (0.1-10 μM) to reach 80% confluence, (e.g., at cell density of 60-100K cells/cm²) followed by treating the cells with Nrf2 activators such as, DMF in the range of 10-250 μm or 4-OI in the range 10-250 μm for 24-72 hours. The conditioned media was further screened to detect the anti-inflammatory molecule expression-s using ELISA. Further, the UC-MSCs primed exosome variant (B′) were isolated from combinatorial priming set (SIRT1 activator and Nrf2 activator in absence of hypoxia). The primed variant of exosomes was then characterized by ELISA detecting the cargo molecules.

3.8 Combinatorial Priming of US-MSCs with the SIRT1 activator and the NRF2 activator in presence of hypoxia—(EXO VARIANT B′)

UC-MSCs were cultured were cultured following the protocol as described in example 1.3, followed by priming with SRT-2104 in a range of 0.00001-0.01 μM or trans-Resveratol (RSV) in a range of 0.1-10 μM. Hypoxia was generated in cycles of hypoxia/normoxia (8-20 cycles with an interval of 30-90 min). For hypoxia, oxygen concentration was 0.5-10% and whereas, in normoxia, oxygen concentration was 14-22%. The cells were then treated with Nrf2 activators DMF in the range of 10-250 μm or 4-OI in the range 10-250 μm, or 24-72 hours after they reached 80% confluence. EV collect exposure, conditioned media collection was performed as described in examples 1.2 and 1.1.2 above. Conditioned media was further screened to detect the anti-inflammatory molecule expressions using ELISA. The UC-MSC primed exosome variant was isolated from combinatorial priming set (SRT1 activator and Nrf2 activator in presence of hypoxia) and primed variant of exosomes were characterized by ELISA for detecting various cargo molecules.

3.9. Combinatorial Priming Protocol of UC-MSCs with SIRT1 Activator (SRT-2104 or Trans-Resveratrol) in Presence of Hypoxia—(EXO VARIANT B′)

UC-MSCs were cultured up to passage 5-6 following the protocol as described in example 1.3, and hypoxia was generated in cycles of hypoxia/normoxia (8-20 cycles with an interval of 30-90 min). For hypoxia oxygen concentration was 0.5-10% and in normoxia, oxygen concentration was 14-22% using tri gas chamber-incubator set up. Once UC-MSCs reached 80% confluence in hypoxic treatment, the cell survivability, HIF-1α, HGF, VEGF and TNF-α expression was checked in the secretome and derived exosome variants. The secretome and exosome profile was compared with UC-MSC derived exosomes where cells were maintained in normoxic condition. Further, SRT-2104 in a range of 0.00001-0.01 μM or trans-Resveratol (RSV) in a range of 0.1-10 μM was used to induce UC-MSCs priming in presence of alternate hypoxia/normoxia cycles. EV collect exposure, conditioned media collection was performed as described in examples 1.2 and 1.1.2 above.

3.10 Combinatorial Priming Protocol of UC-MSC with the Nrf2 Activator (DMF or 4-OI in Presence of Hypoxia) (EXO VARIANT B′)

UC-MSCs were cultured up to passages and hypoxia was generated as described in example 3.8 above. Prior to shifting to EV collect, UC-MSCs were treated with Nrf2 activator DMF in the range of 10-250 μm or 4-OI in the range 10-250 μm for 24-72 hours. EV collect exposure, conditioned media collection was performed according to the protocol described in examples 1.2 and 1.1.2 above. Secretome was characterized by detecting the levels of Nrf2, HIF-1α, HGF, VEGF, sFLT1 by ELISA whereas primed exosome variant (B′) was characterized by ELISA detecting the levels of exosomes cargo molecule expressions, such as Nrf2, HIF-1α, VEGF, sFLT1, IL-10, SIRT1.

3.11 Single Priming Protocol of Stem Cells with all-Trans Retinoic Acid (ATRA)

UC-MSCs/hBM-MSCs were cultured following the protocol as described above. Passage 5-6 was used for primed exosome production and UC-MSC/hBM-MSCs were treated with ATRA inducer with a range of working concentration (0.1-500) μM, 24-72 hours prior to shifting to EV collect. Rooster EV collect incubation, conditioned media collection was performed as described above. After priming, the UC-MSC/hBM-MSC primed conditioned media/secretome was screened with ELISA assay kits to detect expression levels of COX-2, HIF-1, CXCR4, CCR2, VEGF, Ang-2 and Ang-4. Primed exosome variant was then characterized by ELISA assays to detect the expression levels of exosome cargo molecules such as COX-2, HIF-1, CXCR4, CCR2, VEGF, Ang-2 and Ang-4. Based on the results obtained, concentration ATRA was finalized for UC-MSC/hBM-MSC priming.

ATRA is found to increase the viability of MSCs. This was ascertained as the MSCs were treated with various concentrations of ATRA (0.1 μM to 500 μM) for 24 and 48 hours and their viability were examined by MTT assay. The MSC viability was significantly higher in all treated MSCs except for 0.1 μmol/L ATRA. ATRA elevated the PGE2 levels. Pre-treatment of MSC with varying concentration of ATRA (1, 10, 100 μmol/L) significantly increased the PGE2 levels in a dose-dependent manner in MSCs. ATRA increased the expression of genes involved in MSC survival, migration and angiogenesis. The mRNA levels of COX-2, HIF-1, CXCR4, CCR2, VEGF, Ang-2 and Ang-4 was estimated though quantitative real time-PCR and it was elevated in a dose dependent manner when the MSCs were treated with ATRA (1, 10, 100 μmol/L).

3.12. Combinatorial Priming Protocol of Stem Cells with the Nrt2 Activator, SIRT1 Activators, ATRA, in Presence of Hypoxia (EXO VARIANT B′)

UC-MSCs/hBM-MSCs were cultured as described above up to passage (5-6) and hypoxia was generated as described herein. Prior shifting to EV collect, UC-MSCs/hBM-MSCs were treated with the Nrf2 activator (DMF, 4-OI) for 24-72 hours. EV collect exposure, conditioned media collection was performed as described above.

Secretome was characterized by detecting the levels of Nrf2, HIF-1α, HGF, VEGF, sFLT1 by ELISA.

Primed exosome variant was characterized by ELISA detecting the levels of exosomes cargo molecule expressions, such as Nrf2, HIF-1α, VEGF, sFLT1, IL-10, SIRT1.

3.13. Combinatorial Priming Protocol of UC-MSCs/hBM-MSCs with ATRA Inducer in Presence of Hypoxia—(Exo Variant B′)

UC-MSC/hBM-MSCs were cultured as described above up to passage (5-6) and hypoxia was generated as described. Prior shifting to EV collect, UC-MSC/hBM-MSCs were treated with ATRA inducer (0.1-500) μM for 24-72 hours. Rooster EV collect exposure, conditioned media collection was performed as described above. Secretomes were further characterized by detecting the levels of COX-2, HIF-1, CXCR4, CCR2, VEGF, Ang-2 and Ang-4 by ELISA.

Primed exosome variant was characterized by ELISA detecting the levels of exosomes cargo molecule expressions, such as COX-2, HIF-1, CXCR4, CCR2, VEGF, Ang-2 and Ang-4.

3.14. Combinatorial Priming of UC-MSCs/hBM-MSCs with the SIRT1 Inducer and ATRA in Presence of Hypoxia—(Exo Variant B′)

UC-MSCs/hBM-MSCs was cultured as described above following SRT2104 activator or RSV induced priming. Hypoxia was generated in cycles of hypoxia/normoxia (8-20 cycles with an interval of 30-90 min). For hypoxia, oxygen concentration was kept 0.5-10% and in normoxia oxygen concentration is 14-22%. After the cells reach 80% confluence, it was treated with ATRA inducer (0.1-500) μM for 24-72 h. Rooster EV exposure, conditioned media collection was performed as described above.

Conditioned media was screened to detect COX-2, HIF-1, CXCR4, CCR2, VEGF, Ang-2 and Ang-4 expressions using ELISA.

Following optimized exosome isolation protocol. UC-MSC/hBM-MSC primed exosome variants were isolated from combinatorial priming set (SRT1 activator and ATRA inducer in presence of hypoxia). Primed variant of exosomes was characterized by ELISA for detecting various cargo molecules.

Different UC-MSC/hBM-MSC primed variants of exosomes were characterized thoroughly by mass spectrometric analysis to detect the protein profiling and miRNA profiling through technologies such as Nanostring analysis. Functional efficacy of each exosome variant was tested using in vitro assays such as 2D scratch assay, anti-inflammatory assay, anti-fibrosis, pro/anti-angiogenesis and reinnervation assay. Based on the functional efficacy of different variants, the highest scored exosome variant will be selected and continued for in vivo preclinical trials for anti-inflammatory diseases model. LPS induced ARDS induction and bleomycin treated lung injury model will be used for in vivo efficacy testing of the highest scored exosomes.

### Example 4

### Isolation and Purification of Exosomes

The conditioned media was collected from hBM-MSCs. and UC-MSCs, according to the process as described in examples 1.2.2, and 1.3.2, respectively.

The obtained conditioned medium was directly used as secretome or subjected to ultracentrifugation for isolating exosomes. Isolation of exosome from conditioned media/secretome was done by using three methods: (i) Single step ultracentrifugation; (ii) Sucrose based cushion density ultracentrifugation and (iii) Iodixanol density gradient ultracentrifugation.

Isolation of exosome from conditioned media/secretome was done by following three methods are described below.

### 4.1 Sucrose-Based Cushion Density Ultracentrifugation:

The following steps were followed to purify the exosomes using sucrose-based cushion density centrifugation:
(i) After the cells reached 80% confluency, media was removed and cells were washed in 1×PBS (20 mL), followed by adding 260 mL of EV collect media to the flasks and the flasks were then incubated for 72 h at 37° C. and 5% CO₂.
(ii) The supernatant was collected and proceeded with the pre-processing steps as below:
   a. The media was centrifuged at 300×g for 10 min at 4° C., and the supernatant was collected.
   b. The supernatant was centrifuged at 3000×g for 20 min at 4° C., the supernatant was collected.
   c. The supernatant was centrifuged at 13000×g for 30 min at 4° C. and the supernatant was collected.
   d. The media was filtered through a 0.45-micron filter
   e. Next, the media was filtered through a 0.22-micron filter.
(iii) The conditioned media was stored at 4° C. for short term storage (24 hours) or −80° C. for long term storage (1 month). However, in case if processing the media immediately or if frozen, the conditioned media was at 4° C. followed by the protocol as described below:
   a. The conditioned media was centrifuged at 100,000×g for 90 min at 4° C.;
   b. The supernatant was removed carefully. A clear pellet was observed at the bottom of the tube.
   c. The enriched exosomes were transferred on to 30% sucrose (1M) containing ultracentrifuge tube as described in.
   d. The speed was set to 100000 g for 2 h at 4° C. and the acceleration and deceleration were set to zero.
   e. The supernatant was carefully removed, and the exosomes were resuspended in sterile 1X PBS.
   f. The exosomes were aliquoted, and store at −80° C.

### 4.2 Single-Step Ultracentrifugation:

The following steps were followed to purify the exosomes using single-step centrifugation:
(i) After the cells were 80% confluent, the media was removed, and the cells were washed in 1×PBS (20 mL). The PBS was discarded, and 40-45 mL/flask of EV collect media was added to the flasks and then incubated for 72 h at 37° C. and 5% CO₂. The supernatant was collected and proceeded with the pre-processing steps as follows:
   a. The media was centrifuged at 300×g for 10 min at 4° C. and supernatant was collected.
   b. The supernatant was centrifuged at 3000×g for 20 min at 4° C. and supernatant was collected.
   c. The supernatant was centrifuged at 13000×g for 30 min at 4° C. and supernatant was collected.
   d. The media was filtered through a 0.45-micron filter
   e. Next, the media was filtered through a 0.22-micron filter.

The conditioned media was stored at 4° C. for short term storage (24 hours) or −80° C. for long term storage (1 month), whereas, if processing immediately or with thawed samples, following protocol was used:
a. The conditioned media was centrifuged at 100,000×g for 90 min at 4° C.
b. The supernatant was carefully removed. A clear pellet was observed at the bottom of the tube.
c. The pellet was dissolved in PBS/saline, 0.5 mL of crude exosomes was stored at −80° C. for QC.

### 4.3. Iodixanol Density Gradient Ultracentrifugation:

After the cells reached 80% confluent (e.g., 43000-50000 cells/cm²), media was removed, and cells were washed in 1×PBS (20 mL) followed by adding 40-45 mL/flask of EV collect media to the flasks and incubating for 72 h at 37° C. and 5% CO₂. The supernatant was collected and immediately proceeded with the pre-processing steps as described below:
a. The media was centrifuged at 300×g for 10 min at 4° C. and supernatant was collected.
b. The supernatant was centrifuged at 3000×g for 20 min at 4° C. and supernatant was collected.
c. The supernatant was centrifuged at 13000×g for 30 min at 4° C. and supernatant was collected.
d. The media was filtered through a 0.45-micron filter
e. Next, the media was filtered through a 0.22-micron filter.

The conditioned media was stored at 4° C. for short term storage (24 hours) or −80° C. for long term storage (1 month). For processing immediately or with frozen samples, following protocol was followed:
1. The conditioned media was centrifuged at 100,000×g for 90 min at 4° C.
2. The supernatant was removed carefully. A clear pellet was observed at the bottom of the tube.
3. The pellet was dissolved in 36 mL EV collect media (36 mL per 300 mL starting conditioned media), 0.5 ml of crude exosomes was stored at −80° C. for QC.

### Density Gradient Ultracentrifugation (DGUC):

An Iodixanol (IDX) gradient was prepared by floating 3 mL of 10% w/v IDX solution (Sigma #D1556) containing NaCl (150 mM) and 25 mM Tris:HCl (pH 7.4) over 3 ml of 55% w/v IDX solution. Concentrated conditioned media (6 mL) was floated on the top of the IDX cushion and ultracentrifuged using a Beckman Coulter SW 40 Ti rotor for 4.5 h at 100,000×g (4° C.). Twelve fractions (1 mL each) were collected from the top of the gradient on ice and each fraction was collected into pre-chilled 1.5 mL tubes. Fraction-9 was transferred into a fresh ultracentrifuge tube and 11 mL PBS was added to the 1 mL fraction. Ultracentrifugation was repeated at 100,000×g for 4 h in Optima XPN-100 ultracentrifuge using a Beckman Coulter SW 40 Ti rotor at 4° C. The supernatant was discarded and the exosomes were re-suspended in 1 mL PBS. Different aliquots were prepared with 50-100 μL and store at 4° C. for short term (2-3 days) and −80° C. for long term storage.

All of the three methods as described above were followed by a second round of purification using size exclusion chromatography (using Captocore 700 column). The process of exosome purification is described in detail in the pending applications PCT/IN2020/050622. PCT/IN2020/050623, PCT/IN2020/050653, which are incorporated in its entirety in the present disclosure. Although the present example demonstrates the isolation and purification of exosomes from the conditioned media collected from hBM-MSCs, and UC-MSCs, however, it can be contemplated that a person skilled in the art can obtain exosomes from the conditioned media collected from stem cells, including but not limited to, CSSCs, WJMSCs. The stem cells as described herein can be naïve stem cells or the stem cells that are primed with different priming agents as described in example 3, wherein the naïve stem cells (A)/primed stem cells (A′) are used for further for collecting conditioned which can be used for obtaining naïve exosomes (B)/primed exosomes (B′), respectively, by following the protocol as described in the present example.

### Example 5

### Characterization of Exosomes

The harvested or purified exosomes as described in example 4, were characterized by methods such as nanoparticle tracking analysis (NTA), Transmission electron microscopy (TEM), Western blotting, mass spectrometry and analyzing RNA content by real time PCR and RNAseq. The exosome variants that were characterized are naïve exosomes (B), or primed exosomes (B′).

5.1 NTA analysis

Purified exosomes were dissolved in sterile PBS and a separate aliquot (20-50 μl) of exosome fractions are stored at −80° C. Autoclaved milli Q that was filtered with 0.22 pm syringe filter/nuclease free water is used for sample dilution, 1:500 dilution of exosomes sample was used for the NTA data acquisition, 2 μl of exosome sample was taken out from the aliquot after mixing it by pipetting. This was added to 998 μl of milli Q in a 1.5 ml micro centrifuge tubes and mixed multiple times with a 1 ml pipette. Instrument information and data acquisition setting was done by using Nanosight LM 10 from Malvern for the data acquisition with the following setting: camera level 16, gain 3 and three runs, each of 30 second run and threshold.

### 5.2 Transmission Electron Microscopy Imagine of Exosomes

Exosome pellet was fixed with 1 mL of 2.5% glutaraldehyde in 0.1 M sodium cacodylate solution (pH 7.0) for 1 h at 4° C. Fixative was removed and the pellets were rinsed with 1 mL of 0.1 M sodium cacodylate buffer at room temperature. This was repeated thrice with each cycle lasting for 10 min. Samples were fixed with 1 mL of 2% Osmium tetroxide for 1 h at 4° C. The fixative was removed and rinsed thrice with 0.1 M sodium cacodylate buffer every 10 min. Samples were incubated for 10 mins on the shaker using a graded acetone series (50%, 60%, 70%, 80%, 90%, 95%, 100%, respectively). The acetone was removed and the solution of 3:1 acetone: low viscosity embedding mixture was incubated for 30 min to obtain the exosome pellet. Further, 1:1 acetone: low viscosity embedding mixture medium was added again and incubated for 30 min. Medium was removed and 1:3 acetone: low viscosity embedding mixture medium was added followed by incubation for 30 min. Further, the medium was removed and 100% low viscosity embedding mixture was added and incubated overnight at room temperature.

The sample was embedded in pure low viscosity embedding mixture using the embedding mold and baked for 24 h at 65° C. The sections with 60 nm thickness were obtained using an ultra-microtome and double-stained with 2% uranyl acetate for 20 min and lead citrate for 10 min to observe the grids under transmission electron microscopy at 80 kV.

### 5.3: Western Blotting

The western blotting was carried out by following two processes as described below.

### 5.3.1: Protocol 1

Twenty microliters of exosome lysate which corresponds to 0.2 billion of particles were mixed with 20 μl of 2X Laemmli sample buffer (without P mercapto ethanol for CD63. CD9 and CD81). It was heated at 95° C. for 10 min and after vortexing, it was loaded into the gel (12% SDS-PAGE). For Alix and TSG101, 2X Laemmli sample buffer with R mercapto ethanol (e.g., in reducing condition) was used as per the antibody datasheet sample preparation.

### 5.3.2 Protocol 2

Approximately 0.4 2n of exosomes were lyophilized and after that 20 μl of nuclease free water was added to the lyophilized exosomes. This was followed by addition of 20 μl of 2X Laemmli sample buffer and it was heated at 95′C for 10 min. After vertexing, it was loaded into the gel (12% SDS-PAGE).

The PVDF membrane was cut to appropriate size along with the two layers of papers in which it was embedded. The white membrane was separated with the help of forceps and immersed in 50 mL of methanol for activation of the membrane. It was kept for 1 min. and rinsed with distilled water. The activated membrane was then transferred to 50 mL of 1X transfer buffer. The transfer apparatus cassette was cleaned, and the absorbent paper was wetted in approximately 30 ml 1X transfer buffer. One blot absorbent filter paper was placed in the cassette, followed by the membrane, gel and filter paper. A blot roller was used to make sure the bubbles were removed between the blot and gel. The transfer was set for 60 min at 25 V and 2.5 A.

After completion of the transfer, the PVDF membrane was carefully removed using forceps and incubated in a blocking solution (5% non-fat milk solution in 50 mL 1X TBST) for 1 h on the shaker at room temperature. The PVDF membrane was then removed from the blocking solution using forceps and given a 1X TBST wash for 10 min on shaker at RT. The membrane was carefully cut into stripes according to the protein size using clean forceps.

The PVDF stripes were incubated in respective primary antibodies (diluted in 0.1% BSA in 1X TBS) with respect to the protein of interest overnight at 40° C. on the shaker. The PVDF membranes were removed next day from the primary antibodies and rinsed 6 times in 1X TBST buffer (each rinse for 5 mins). The primary antibodies were reused by storing them at −20° C. The membranes were incubated with HRP conjugated secondary antibody (diluted in 0.1% BSA in 1X TBS) for 2 h at RT on the shaker. Membranes were washed 6 times with 1X TBST buffer (each rinse for 5 mins) and after the last wash, the membrane was kept in the transfer buffer prior to developing. Blots were developed using ECL chemiluminescent reagent in dark (active reagent was prepared as per the guidelines of the manufacturer).

### 5.4 Mass Spectrometry

### 5.4.1 Sample Preparation:

The samples were thawed at 2-8° C. and 25 μL of sample was mixed with 25 μl of lysis buffer (0.1% Triton-X100, 100 mM DTT, 150 mM Tris-HCl pH 8.0) followed by incubation for 1 hour at room temperature and brief sonication. The obtained samples were loaded in three different lanes on pre-casted SDS-PAGE gel (Invitrogen NuPage 4-12% Bis-Tris Gradient Gels). A brief electrophoresis (<10 minutes) was carried out at constant voltage to remove the detergent from the protein samples. Protein bands were visualized using Gel-Code blue stain. An in-gel trypsin protein digestion method that includes reduction and alkylation of Cysteine residues was utilized to digest the protein. The obtained tryptic peptides were pooled and clarified using C18 zip-tips. Zip-tip eluates were concentrated close to dryness and were dissolved in 8 μL of 0.1% FA. Three replicate injections of 2 μL each were performed for identification of protein.

### 5.4.2 Mass-Spec Based Protein Identification

Using a nanoflow set-up, tryptic peptides were separated on a reverse phase liquid chromatographic column through a linear gradient of 0.1% formic acid (FA) and acetonitrile developed over a period of 110 minutes (total run time 140 minutes). Data was collected in well-optimized conditions in data-dependent mode. In the optimized conditions standard, He—La cell Trypsin digest at 2 μg load provided identification of 6000 proteins. The acquired MS and MS/MS data of the test-sample were searched against Human Proteome database using Maxquant Software. Protein Identification was performed with the following criteria: (a) Trypsin digested pephides with 4 missed cleavages allowed, (b) peptide tolerance 10 ppm, (c)≥S1 unique peptide, (d) FDR<1% and (e) Fixed Modification—carbamidomethylation of cysteine and variable modification—Oxidation of Methionine.

Table 9 shows a list of protein biomarkers that could be present as cargo in primed exosome variants and may be detected using one or more standard methods of protein detection such as Western Blot, ELISA, or Mass Spec.

TABLE 9

| Proteins | Significance |
|---|---|
| SIRT1 | Promotes regeneration and survival |
| SIRT6 | Involved in molecular pathways |
| | related to aging, including DNA |
| | repair, telomere maintenance, |
| | glycolysis and inflammation. |
| HIF-1α | Promotes MSC proliferation and |
| | survival. Improves their antioxidant |
| | ability |
| SFLT1 | Anti-angiogenesis |
| NRF2 | Anti-inflammatory |
| FGF 21 | Promotes nerve regeneration through |
| | suppressing oxidative damage |
| MANF | Neurogenesis |
| Angiopoietin | Promotes angiogenesis |
| BDNF | Neuronal development, function |
| | and survival |
| COX-2 | Induces inflammation |
| FGF-1 | Plays an important role in the |
| | regulation of cell survival, cell |
| | division, angiogenesis, cell |
| | differentiation and cell migration. |
| GDNF | Neurotrophic factor that enhances |
| | survival and morphological |
| | differentiation of dopaminergic |
| | neurons and increases their |
| | high-affinity dopamine uptake |
| HGF | Involved in cell invasion, |
| | proliferation and angiogenesis |
| IDO | Immunosuppressive and |
| | anti-inflammatory |
| IGF-1 | Promotes synaptic plasticity, |
| | enhances nerve growth and triggers |
| | anti-apoptotic-mediated signaling |
| | cascades |
| IL-10 | Anti-inflammatory |
| KGF | Anti-inflammatory |
| NGF | Reinnervation |
| SDF-1 | Anti-fibrotic |
| TGF-β | Anti-inflammatory |
| Lin28 | Promotes regeneration |
| a-Klotho | Anti-inflammatory |
| Sox2 | Neurogenesis |
| Tet1 and Tet 2 | Ten-eleven translocation family |
| | demethylase that regulates epigenetic |
| | responses. Maintain BM-MSCs self- |
| | renewal and bone homeostasis. It |
| | controls the exosomes released from |
| | BM-MSCs. |
| AMPK | Master regulator of cellular energy |
| | metabolism. |
| NOX 4 | Generates reactive oxygen species |
| | and controls cellular proliferation. |
| PGC-1alpha | Antioxidant stress, regulates |
| | mitochondrial function and regulating |
| | the metabolism of glucose and lipid |
| ApoE4 | Regulates lipid metabolism, induces |
| | Alzheimer. |
| NF-kβ | Nuclear factor-κB (NF-κB) represents |
| | a family of inducible transcription |
| | factors, which regulates a large |
| | array of genes involved in different |
| | processes of the immune and |
| | inflammatory responses. |
| IL-6 | Inflammatory |
| VEGF | Pro angiogenic |
| IL-1β | Inflammatory |
| IFN-γ | Inflammatory |
| IL-2 | Inflammatory |
| TNF -α | Inflammatory |
| LIF | Promotes angiogenic potential |
| p53 | Cellular senescence and apoptosis |
| Alix | Member of ESCORT protein family |
| | and during biogenesis of exosomes |
| | transport into the exosomes vesicles. |
| CD63 | Member of tetraspanin family protein |
| | and it's a surface marker of exosomes |
| CD81 | Member of tetraspanin family protein |
| | and it's a surface marker of exosomes |
| CD9 | Member of tetraspanin family protein |
| | and it's a surface marker of exosomes |
| HSp70 | Heat shock protein used as exosome |
| | marker |
| TSG101 | Member of ESCORT protein family |
| | and during biogenesis of exosomes |
| | transport into the exosome vesicles. |
| Angiostatin | Anti-angiogenesis |
| BMP-7 | Anti-fibrosis |
| CCL5 | Anti-inflammatory |
| CXCL12 | Angiogenesis |
| Endoglin | Angiogenesis |
| FAS | Induction of apoptosis |
| FLOT1 | Affects the formation and release |
| | of exosomes |
| FLOT2 | Affects the formation and release |
| | of exosomes |
| Galectin 1 | Responsible for adherence on the |
| | cell surface and extracellular |
| | matrix proteins. |
| TSG-6 | Anti-inflammatory |
| Fibronectin | Pro-fibrotic, promotes cell survival |
| | and migration |
| CD73 | Immune suppression |
| PGE2 | Anti-inflammatory |
| Stanniocalcin 1 | Immunomodulatory and |
| | pro-angiogenic |
| PAPP-A | Release IGF-1, anti-apoptotic |
| Progranulin | Promotes peripheral nerve |
| | regeneration and reinnervation |
| DLK (Dual Leucine | Nerve Regeneration and anti-aging |
| zipper kinase) | |
| IL-38 | Anti-inflammatory |
| Thymosin-β4 | Wound healing and regeneration |
| Opioid growth | Cellular renewal, wound healing, |
| factor (OGF), or | and angiogenesis |
| (Met5)-enkephalin | |
| Connective tissue | ECM remodeling and Fibrosis |
| growth factor (CTGF) | |
| Alpha | Inflammatory (Acute phase) |
| 1-Antitrypsin | |
| Timp2 | Anti-angiogenic |
| Alpha1- | Inflammatory |
| antichymotrypsin | |
| Thrombospondin-1 | Anti-angiogenic |
| (TSP-1) | |
| Epiregulin | Wound healing and regeneration, |
| | Pro-angiogenic |
| α2-macroglobulin | Inflammatory |
| TEAD 3 | Muscle regeneration |
| NFIX | Proliferation and regeneration |
| RFX2 | Neuronal development and regeneration |
| SOX 10 | Neuronal development and regeneration |
| α1-acid glycoprotein | Inflammatory (Acute phase) |
| (α1AG) | |
| ETV2 | Reprogramming or regeneration |
| | (hemato to endothelial lineages) |
| NEUROD2 | Neuronal development (regeneration) |
| C-reactive protein | Inflammatory (Acute phase) |
| (CRP) | |
| Serum response factor | Neuronal development (regeneration) |
| (SRF) | |
| PAX5 | Regeneration of B cells and oncogenic |
| Ceruloplasmin | Inflammatory (Acute |
| | phase)/angiogenesis |
| MEF 2 | Cardiac, skeletal muscle and neuronal |
| | development |
| LARP1 | Ageing and Longevity |
| Angiogenin | Angiogenesis |
| Fidgetin | Nerve regeneration |
| Cyr61 (Secretome from | Pro angiogenesis |
| MSC) Cysteine rich | |
| protein 61 | |
| RNASE4 (Secretome | Angiogenic |
| from ADSCs) | |
| FTY720P (Fingolimod) | Nerve regeneration |
| Secretome from | |
| schwann cells | |
| MMP-2 | Regulators of tissue fibrosis |
| MMP-7 | Regulators of tissue fibrosis |
| MMP-9 | Regulators of tissue fibrosis |
| Perlecan | Plays role in the structure of the |
| | corneal epithelium |
| Versican | Facilitate and control open flow |
| | channels in the trabecular meshwork |
| Hyaluronan | Fibrosis |
| Decorin, biglycin, | Corneal wound healing and homeostasis |
| fibromodulin, mimecan, | |
| keratocan, and lumican | |
| MMP 14 (exosome from | Cleaves and inhibits sFLT, involved |
| corneal fibroblast) | in VEGFA induced corneal |
| | neovascularisation |
| Epiregulin | Regulate the expression of cytokines |
| | and chemokines to reduce an excessive |
| | accumulation of PMN cells |
| HMOX1 | Tumour suppressive |
| Semaphorin 7A | Angiogenesis |
| Klf5 | Corneal epithelial homeostasis |
| | by regulating the expression of |
| | desmosomal components |
| Human positive | PC4 is involved in histone acetylation |
| cofactor (PC4) | and serves as a potential target to |
| | improve proteostasis and delay ageing |
| Peroxisome proliferator- | Anti-inflammatory, anti-thrombotic, |
| activated receptor | regulates the expression of genes |
| (PPAR)-alpha | involved in fatty acid beta- |
| | oxidation, energy homeostasis. |
| Mammalian target of | Tumour suppressive, regulates cell |
| Rapamycin (mTOR) | proliferation, autophagy, and apoptosis |
| Ubiquitin-specific | Apoptosis and integrin stabilization |
| protease 10 (USP10) | |
| Protein kinase C (PKC) | Reduction of signaling results in |
| | anti-aging |
| CXCL9 | Tumour suppressive (paracrine axis), |
| | cancer proliferation and metastasis |
| | (autocrine axis) |
| PDK1 | Oncogene; inhibition results in |
| | anti-aging |
| HRAS (H-Ras-GTPase)-Harvey | HRAS (H-Ras-GTPasc)- Harvey rat |
| rat sarcoma viral oncogene | sarcoma viral oncogene |
| aka Transforming protein p21 | aka Transforming protein p22 |
| HRAS (H-Ras-GTPase)- Harvey | Cell repair, growth regulation |
| rat sarcoma viral oncogene | |
| aka Transforming protein p22 | |
| HRAS (H-Ras-GTPase)- Harvey | Cell cycle regulation |
| rat sarcoma viral oncogene | |
| aka Transforming protein p23 | |
| BMI1(B lymphoma Moloney | Cell proliferation, differentiation |
| murine leukemia virus | and senescence; DNA damage repair |
| integration site 1) (oncogene) | |
| E2F1 (E2F Transcription | Cell cycle regulation and apoptosis |
| factor1) | |
| CTNNB1 (Catennin Beta 1) | Signal transduction, Neg regulation |
| | of apoptotic process |
| ERCC5 (Excision Repair 5, | DNA damage repair, Neg regulation |
| Endonuclease) | of apoptotic process |
| PDGFB (Platelet derived | Growth regulation, Cell proliferation |
| growth factor B) | |
| IGF1R (Insulin like growth | Neg regulation of apoptotic process |
| factor 1 receptor) | |
| DGAT1 (Diacylglycerol | Metabolism |
| O-Acyltransfersae 1) | |
| PLCG2 (Phospholipase | Signal transduction |
| C-Gamma-2) | |
| GHR (Growth hormone | Growth regulation |
| receptor) | |
| FGF23 (Fibroblast | Growth regulation |
| Growth factor 23 | |
| PDGFRB (Platelet derived | Growth regulation |
| growth factor receptor beta) | |
| GRN (Granulin precursor | Metabolism |
| or Progranulin) | |
| PIK3CB (Phosphatidylinositol- | Signal transduction |
| 4,5-bisphosphate 3-kinase | |
| catalytic subunit beta isoform) | |
| IRS1 (Insulin receptor | Signal transduction |
| substrate 1) | |
| HBP1(HMG-Box Transcription | Transcription regulation |
| factor 1) | |
| ERCC6(DNA Excision repair | DNA damage repair |
| protein) or CS-B Protein | |
| HESX1 (Homeobox expressed | Transcription regulation |
| in ES cells 1) | |
| PTPN1 (Tyrosine-Protein | Signal transduction |
| phosphatasenon-receptor | |
| type 1) | |
| CDC42(Cell Division | Cell cycle |
| cycle 42) | |
| DBN1 (Drebrin) | Signal transduction |
| HELLS (Lymphoid specific | DNA damage repair |
| Helicase) | |
| FOXO4 (Forkhead box | Transcription regulation |
| protein O4) | |
| EMD (Emerin) | Growth regulation |
| SHC1 (SHC Transforming | Growth regulation |
| protein 1) | |
| PPARA (Peroxisome | Metabolism |
| proliferator activated | |
| receptor alpha) | |
| GHRH (Growth hormone | Growth regulation |
| releasing hormone) | |
| Overlapping genes | |
| BCL2 (B-cell lymphoma 2) | Apoptosis |
| oncogene | |
| EGR1 (Early growth | Growth regulation |
| response protein 1) | |
| VCP (Valosin containing | DNA damage repair |
| protein) | |
| NCOR1(Nuclear receptor | Transcription regulation |
| corepressor 1) | |
| STAT5B (Signal transducer | Transcription regulation |
| and activator of | |
| transcription 5B) | |
| ERBB2(Erythroblastic | Cell division, Cancer |
| oncogene B) or CD340 | |
| ERCC3 (Excision repair | DNA damage repair |
| 3, TFIIH core complex | |
| helicase subunit) | |
| IGF1 (Insulin like growth | Growth regulation |
| factor 1) | |
| ARNTL (Aryl Hydrocarbon | Transcription regulation |
| receptor nuclear translocator | |
| like) | |
| ATM (Ataxia-Telangicctasia | Cell division, Cancer |
| Mutated) | |
| INSR (Insulin receptor) | Growth regulation |
| STAT5A (Signal Transducer | Transcription regulation |
| and activator of | |
| transcription 5A) | |

### 5.5 Exosomal RNA Isolation

### 5.5.1 Exosome RNA Isolation Protocol

RNA extraction was performed from the naïve BM-MSCs derived exosomes using the RNeasy Mini kit from Qiagen according to the manufacturer's protocol. Five billion of exosome was considered to isolate Exosomal RNA. RNA quantification was performed in Nanodrop and Qubit and Qubit microRNA assay was used to check the presence of miRNA/small RNA molecules.

5.5.2 Exosomes RNA Isolation from 10 Billion Lyophilized Exosomes

RNA extraction was carried out using miRVana miRNA Isolation Kit (Cat #: AM1560) and 4 elutions were mad with a volume of 25 μl, 25 μl, 50 μl and, 50 μl respectively. RNA quantification was performed in Nanodrop and Qubit and Qubit microRNA assay was used to check the presence of miRNA/small RNA molecules.

5.5.3 Exosomes miRNA Profiling

Different elute of exosome RNA extracted were run in Bio analyzer to check the presence of small RNA and miRNA. Elutes were pooled together and were prepared for performing miRNA profiling using the NanoString platform. All the processes were carried out based on manufacturer's instructions.

Table 10 shows the list of miRNA analyzed in primed exosome variants.

TABLE 10

| miRNA | Significance |
|---|---|
| miR-1246 | Cellular proliferation |
| miR-126 | Angiogenesis |
| miR-133b | Promote neurite remodeling |
| miR-145 | Anti-inflammatory |
| miR-146 | Tumour suppressive |
| miR-155 | Promotes inflammation |
| miR184 | Drug resistance |
| miR-205 | Angiogenesis |
| miR-21 | Angiogenesis |
| miR-23b | Tumour suppressive |
| miR-29c | Anti-fibrotic |
| miR-455-3p | Anti-inflammatory |
| Let-7 | Tumour suppressive |
| lncRNA | Anti-inflammatory |
| Malat1 | |
| miR-122 | Increased chemosensitivity of HCC cells |
| miR-101 | Tumour suppressive |
| miR-127-3p | Myogenic differentiation |
| miR-130 | Tumour suppressive |
| miR-150 | Tumour suppressive |
| miR-15a | Inhibits cell proliferation, promotes apoptosis |
| | of cancer cells, and suppresses tumorigenicity |
| miR-15b | Inhibitors of chemotherapy-induced |
| | EMT and tumor metastasis |
| miR-16 | Tumour suppressive |
| miR-181a | Tumour suppressive |
| miR-195 | Tumour suppressive |
| miR-451a | Tumour suppressive |
| miR-497 | Anti-tumor and anti-angiogenic effect |
| miR-145 | Tumour suppressive |
| miR-361-5p | Tumour suppressive |
| miR-200b | Anti-angiogenesis |
| miR-100 | Anti-angiogenesis |
| miR-29 | Anti-fibrotic function |
| (b and c) | |
| miR-22 | Pro-fibrotic |
| miR-210 | Pro-angiogenic |
| miR-10b | Promotes cell invasion |
| miR-126-5p | Reverse the expression pattern of aging |
| miR-9 | Fibroblastic |
| miR-105 | Induction of metastatic and vascular permeability |
| miR-214 | Suppress senescence |
| miR-136 | Anti-aging and myocardial repair |
| miR-140 | Wound repair and regeneration |
| miR-181-5p | Anti-fibrosis |
| miR-223 | Anti-inflammatory |
| miR-494 | Induce muscle regeneration via |
| | increased angiogenesis and myogenesis |
| miR-22 | Pro-fibrotic |
| miR-10b | Promotes cell invasion |
| miR-143 | Induce stemness and epithelial-mesenchymal transition |
| miR-378c | phenotype in cancer cells |
| miR-499a-5p | Enhanced cell proliferation, migration |
| | and EMT via mTOR pathway |
| miR-98-5p | Increased cell proliferation and decreased apoptosis. |
| | Promoted cisplastin resistance and downregulated |
| | CDKN1A |
| miR-3202 | Regulation of hyperglycemia induced endothelial cells |
| | by targeting Fas apoptotic inhibitory molecule 2 |
| miR-32-5p | Multidrug resistance by targeting PTEN and |
| | activating the PI3K/Akt pathway |
| miR-363-5p | Inhibited cancer cell migration and specific |
| | complementary predictor for LN metastasis. Regulates |
| | platelet derived growth factor B, which in turn |
| | contribute to the induction and maintenance of EMT |
| | in breast cancer |
| miR-548j-3p | Attenuates inflammatory response in amnion |
| | epithelial cells |
| miR-219a-5p | Tumour suppressor |
| miR-450a-5p | Regulator of ADSC differentiation into adipocytes |
| miR-516b | Tumour suppressor |
| miR-495 | Induced senescence |
| miR-218-5p | Affect matrix synthesis gene expression and |
| | chondrocyte proliferation and apoptosis. |
| miR-101 | Tumour suppressor (miR-101 inhibited cell |
| | proliferation directly by decreasing the expression |
| | of enhancer of zeste homolog 2 (EZH2) in |
| | lung cancer) |
| miR-132 | Regulates endothelial cell behavior during |
| | angiogenesis (pro-angiogenesis) |
| miR-196a | Promotes cell proliferation and inhibits cell |
| | apoptosis by targeting CDKN1B and ING5 in |
| | the head and neck cancer microenvironment. |
| miR-1246 | promote tumor angiogenesis via targeting the |
| | promyelocytic leukemia protein and the Smad |
| | signaling pathway in endothelial cells |
| miR-515-5p | Anti-proliferation, anti-invasion and pro-apoptosis |
| | effects by targeting EEF2 in NSCLC cells (non- |
| | small cell lung cancer). |
| miR-342-5p | Cardioprotective molecule |
| miR-574-5p | Associated with enhanced synthesis of synthesis of |
| | prostaglandin E2 (14), an important pro-inflammatory |
| | lipid mediator which mediates inflammation in RA |
| miR-4787-3p | Associated with lymph node metastasis |
| miR-25-3p | Induction of vascular permeability and angiogenesis |
| miR-193a | Causes cell cycle G1 arrest and cell proliferation |
| | repression through targeting of Caprin1 |
| miR-567 | Suppressed autophagy and reversed chemoresistance by |
| | targeting ATG5 |
| miR-548a-5p | Anti-inflammatory; inhibits the proliferation and |
| | activation of pTHP-1 cells by regulating the |
| | TLR4/NF-κB signaling pathway. |
| miR-483-3p | Regulate keratinocyte migration and proliferation |
| | in wound healing through down-regulation of MK2, |
| | MKI67, and YAP1 |
| miR-24 | Cardioprotection, reduced infarct size after |
| | I/R injury, which was associated with the |
| | inhibition of pro-apoptotic genes and |
| | increase in anti-apoptotic genes. |
| miR-204 | Decline in cell proliferation, induced cell |
| | cycle G1-arrest, and inhibited migration |
| miR-509-3p | Suppressed the proliferation of of cell, induced |
| | cell apoptosis and inhibited cell migration in |
| | vitro in renal cell carcinoma |
| miR-424 | Promotes angiogenesis, tumour suppressive |
| miR-297 | Multi-drug resistance |
| miR-92b | Differentiation and proliferation |
| miR-346 | Anti-inflammatory |
| miR-150-5p | Tumour suppressive |
| miR-204-p | Tumour suppressive and suppression of vascularization |
| miR-30a-5p | Facilitates retinal angiogenesis |
| miR-487b | Tumour suppressive |
| miR-34c-5p | Tumour suppressive and apoptosis |
| miR-34b | Tumour suppressive |
| miR-663 | Proliferation and cell cycle regulation |
| miR-503 | Proliferation and cell cycle regulation |
| miR-181c | Pro-inflammatory |
| miR-595 | Tumour suppressive |
| miR-200a | Migration and differentiation |
| miR-7-1 | Tumour suppressive |
| miR-149 | Proliferation, vascular, lymph node and nerve invasion |
| miR-188-3p | Anti-inflammatory |
| miR125b-5p | Tumour suppressive |
| miR-143-3p | Tumour suppressive |
| miR-155-5p | Reduced corneal permeability and accelerated the |
| | recovery of corneal epithelial wounds by decreasing |
| | the expression of MLCK and phosphorylation of MLC |
| | and by remodeling tight junctions. |
| miR-21-5p | Pro-angiogenic |
| miR-1910-3p | Proliferation and metastasis |
| miR-124-3p | Tumour suppressive |
| Let-7c | Anti-fibrotic |
| Let-7b-5p | Tumour suppressive |
| miR-183-5p | Angiogenesis |
| miR-375 | Osteogenic differentiation, enhance bone regeneration |
| | (in vitro and in vivo) |
| miR-99b | Increase Fgf21 expression, increase glucose tolerance |
| | (in vivo) |
| miR-34a, | Reversed Pulmonary hypertension in monocrotaline mice |
| miR-122, | model (anti-inflammatory, anti-proliferative effect) |
| miR-124, and | |
| miR-127 | |
| miR-27b-3p | Anti-fibrotic |
| miR-19a | Cell survival (activates AKT and inhibit |
| | JNK/caspase3 pathway) |
| miR-144 | Anti-apoptotic |

Table 11 shows the list of mRNA analyzed in primed exosome variants

TABLE 11

| mRNA | Significance |
|---|---|
| BMP15 | Oocyte-derived growth and differentiation factor is a |
| | critical regulator of folliculogenesis and GC activities. |
| COL4A2 | Anti-angiogenic |
| CXCR7 | Affect cell survival, proliferation and chemotaxis |
| IL1RN | Immunomodulatory |
| IRF6 | DNA-binding transcriptional activator. Key determinant |
| | of the keratinocyte proliferation-differentiation switch |
| | involved in appropriate epidermal development. Plays a |
| | role in regulating mammary epithelial cell proliferation |
| ADAM15 | Tumour inhibitory activity |
| ADM2 | Member of the calcitonin gene-related peptide |
| | (CGRP)/calcitonin family of hormones that play a |
| | role in the regulation of cardiovascular homeostasis, |
| | prolactin release, anti-diuresis, anti-natriuresis |
| BCL6B | Tumour suppressive |
| BDH2 | Tumour suppressive |
| CDC14B | Microtubule-bundling and -stabilizing protein, whose |
| | regulated subcellular localization may help modulate |
| | spindle and microtubule dynamics in mitosis |
| CLEC2A | Role in modulating the extent of T-cell expansion. |
| | Enhances the expansion of TCR-stimulated T-cells by |
| | increasing their survival through enhanced expression |
| | of anti-apoptotic proteins. |
| CRLF1 | Complexes with CLCF1 to form a heterodimeric |
| | neurotropic cytokine that plays a crucial role |
| | during neuronal development. |
| | May also play a regulatory role in the immune system |
| CTNNA1 | Role in cell adhesion process by connecting cadherins |
| | located on the plasma membrane to the actin filaments |
| | inside the cell. |
| EPX | Mediates tyrosine nitration of secondary granule |
| | proteins in mature resting cosinophils. Shows significant |
| | inhibitory activity towards Mycobacterium tuberculosis |
| | H37Rv by inducing bacterial fragmentation and lysis. |
| FUT3 | Function in embryogenesis, tissue differentiation, tumor |
| | metastasis, inflammation, and bacterial adhesion |
| IBSP | Binds tightly to hydroxyapatite. Appears to form an |
| | integral part of the mineralized matrix. Probably |
| | important for cell-matrix interaction. Promotes |
| | Arg-Gly-Asp-dependent cell attachment. |
| LTA4H | Protein encoded by this gene is an enzyme that contains |
| | both hydrolase and aminopeptidase activities. The |
| | hydrolase activity is used in the final step of the |
| | biosynthesis of leukotriene B4, a proinflammatory |
| | mediator. The aminopeptidase activity has been shown |
| | to degrade proline-glycine-proline (PGP), a neutrophil |
| | chemo attractant and biomarker for chronic obstructive |
| | pulmonary disease (COPD). |
| MAGED2 | Regulates the expression, localization to the plasma |
| | membrane and function of the sodium chloride |
| | cotransporters SLC12A1 and SLC12A3, two key |
| | components of salt reabsorption in the distal renal |
| | tubule |
| MSN | ERM proteins appear to function as cross-linkers between |
| | plasma membranes and actin-based cytoskeletons. Moesin is |
| | localized to filopodia and other membranous protrusions |
| | that are important for cell-cell recognition and signaling |
| | and for cell movement. |
| NIN | This gene encodes one of the proteins important for |
| | centrosomal function. This protein is important for |
| | positioning and anchoring the microtubules minus-ends |
| | in epithelial cells. |
| RAB5A | Binds to a variety of effector proteins to regulate |
| | cellular responses such as intracellular membrane |
| | trafficking, from the formation of transport vesicles |
| | to their fusion with membranes. RAB5A is required |
| | for the fusion of plasma membranes and early endosomes. |
| | Required for the exosomal release of SDCBP, CD63, |
| | PDCD6IP and syndecan |
| RBL-1 | Tumour suppressive |
| S100A13 | Involved in the regulation of a number of cellular |
| | processes such as cell cycle progression and |
| | differentiation. In smooth muscle cells, this protein |
| | co-expresses with other family members in the nucleus |
| | and in stress fibers, suggesting diverse functions in |
| | signal transduction. Required for the copper-dependent |
| | stress-induced export of IL1A and FGF1. |
| SCNN1G | Plays an essential role in electrolyte and blood pressure |
| | homeostasis, but also in airway surface liquid homeostasis |
| SENP2 | Tumour suppressive |
| TOPORS | Functions as an E3 ubiquitin-protein ligase and as an E3 |
| | SUMO1-protein ligase. Probable tumor suppressor involved |
| | in cell growth, cell proliferation and apoptosis that |
| | regulates p53/TP53 stability through ubiquitin-dependent |
| | degradation. |
| hTERT | Regenerative potential at the wound side |
| TGF-beta | Tumor Suppression |
| R2 | |

### 5.6 Real Time PCR

Total RNA isolated using RNAeasy and mirVana™ (n=3), was DNase treated using Turbo DNase Free kit (Ambion), miRCURY™ LNA™ micro-PCR System was used for first strand cDNA synthesis and real-time PCR according to manufacturer's protocol. In brief, each cDNA synthesis was performed in duplicates using a fixed volume of total RNA, miR-451 specific reverse primer (Gene ID: 574411), and first strand cDNA synthesis kit reagents and was incubated for 30 min at 50° C. followed by 10 min at 85° C. Each cDNA sample was then diluted 1:10 and used in duplicates with miRCURY™ LNA™ SYBR® Green master mix, the Universal primer and the LNA™ PCR miR-451 specific primer. PCR was performed for 10 min at 95° C.; 10 s at 95° C.+5 s at 60° C. for 40 cycles and finalized by a melting curve 5 s for each 0.5° C. Control samples was run in parallel. The CFX96 real-time PCR detection system (Bio-Rad, Hercules. CA, USA) was utilized for both cDNA and real-time PCR reactions.

### Example 6

### Functional Characterizations of Exosomes

Exosomes were functionally characterized by assessing the following assays: a) Scratch assay (wound healing capability); b) Anti-inflammatory assay; c) Anti-fibrosis assay; d) Reinnervation assay; e) Angiogenesis (anti/pro) assay

### 6.1 Scratch Assay

Immortalized human corneal epithelial cells (hTCEPI) were used for the 2D scratch assay, hTECPI cells were seeded in tissue culture treated culture dishes at a density of 5000 cells/cm² in serum free media and cells were allowed to grow till it becomes confluent prior to creating a scratch across the centre of the well. Media was removed and cells were washed with 1× PBS to get rid of floating cells followed by adding the media containing (1-20)×10⁸ exosomes/mL to each well. The cells were incubated at 37° C., 5% CO₂ and scratch closure was assessed every 6 h till complete closure. Images of the cells were captured at different timepoints (every 6 h) and wound width was quantified using ImageJ. Controls taken were either medium only (no exosomes) or exosomes depleted control/media.

6.2 Anti-inflammatory assay

RAW264.7 macrophage cells were seeded in tissue culture dishes at a density of 5000 cells/cm² in complete media (RPMI+10% FBS) and cells were allowed to grow till 80% confluent. The cells were starved for 16 h in serum free media and stimulated with LPS (10 ng/mL) in the presence or absence of (1-20) X10⁸ exosomes/mL supplemented in the media for 4 h. The media was collected post treatment and secreted cytokine levels were measured by ELISA. Additionally, the cells were lysed, and transcript levels of cytokines were measured by qPCR to complement the secreted protein levels. Controls taken were either medium only (no exosomes) or exosomes depleted control/media.

### 6.3 Anti-Fibrosis Assay

Human corneal epithelial cells were seeded in tissue culture treated culture dishes at a density of 5000 cells/cm² in serum-free media and cells were allowed to grow till 80% confluent. Cells were treated with TGF-β (10 ng/mL) for 24 h in the presence or absence of (1-20) X10⁸ exosomes/mL supplemented in the media. The extent of induction of fibrosis was assessed by characterizing the expressions of collagen 1, alpha-smooth muscle actin, and fibronectin by immunofluorescence. Controls taken were either medium only (no exosomes or exosomes depleted control/media.

### 6.4 Reinnervation Assay

PC12 cells were seeded on collagen coated plates at a seeding density of 5000 cells/cm² and media was replaced with serum free media after 24 hours of cells seeding, with the treatment of (1-20) X10⁸ exosomes/mL. Images were captured at 24 hours intervals up to 3-5 days. Controls taken were either medium only (no exosomes or Exosomes depleted control/media as negative control whereas NGF (20 ng/mL) was taken as positive control.

### 6.5 Angiogenesis (Anti/Pro) Assay

Human vascular endothelium cells (HUVECs) or coronary artery endothelial cells (CAECs) were used for the assay. HUVECs were grown for 24 hours in DMEM supplemented with 10% FBS, 2 mM L-glutamine, 1 mM sodium pyruvate, 100 U/mL penicillin, and 100 μg/mL streptomycin. One day prior to assay, HUVEC cells were serum starved as follows: aspirate media from the cells, add reduced serum media of DMEM supplemented with 0.2% FBS, 2 mM L-glutamine, 1 mM sodium pyruvate, 100 U/mL penicillin, and 100 μg/mL streptomycin and the cells were grown for an additional 24 hours. Followed by this step, 300 μL of Matrigel (growth factor reduced) was added into a 24 well plate and allowed to solidify at 37° C. for 30 min. HUVECs (2×10⁴/well) in serum free media was suspended in VEGF supplemented media (conc.) either in the presence or absence of exosomes (1-20)×10⁸ for 24 h. Cells were stained with Cell Tracker™ Green CMFDA following the manufacturer's instructions and the tube formation was detected using immunofluorescence staining.

### 6.6. Cell Transformation Assay

Mouse 3T3 cells in DMEM/HAM's F-12 containing 3 g/l D-glucose, 5% fetal bovine serum and 1% penicillin/streptomycin was used for the cell transformation assay, 3T3 cells (5000 cells/well) were seeded into Corning® Primaria™ 6-well plates and cultured under standard conditions (37° C., 5% CO2, 95% humidity) for 42 days. Cells were treated with the samples 24 h after seeding and the media was changed 3 days after treatment. In addition, tumour promoter TPA (12-O-Tetradecanoyl-phorbol-13-acetate, 0.3 g/ml, Sigma #79346) was added on day 8, 11, 15, 18 until day 21. After 42 days, cells were washed twice with PBS, fixed with PBS/methanol (50:50) for 3 min and 100% ice-cold methanol for 10 min, and finally, washed twice with methanol.

Controls that were taken for the purpose of the present disclosure: (i) medium only (no exosomes); (ii) exosomes depleted control/media as negative control; (iii) VEGF was taken as positive control.

### Example 7

### Investigating the Efficacy of Exosomes for Preclinical Trials

Based on the functional efficacy of different variants, as described in example 8, the highest scored exosome variants were selected and continued for in-vivo preclinical trials for anti-inflammatory diseases model, wherein the application of the selected exosome variants were used for the regeneration of specific tissue, i.e., avascular and vascular tissues.

### 7.1 In Vivo Efficacy Studies

Preclinical efficacy studies were undertaken in multiple in-vivo acute respiratory distress syndrome (ARDS) models as described below. MSC-derived Exosomes was used in one or more in-vivo murine models of COVID-19 associated ARDS:
LPS induced lung injury model or
Bleomycin induced lung fibrosis model

### 7.2 Animal Model

The mouse model (8-10 weeks old) was used for the purpose of in-vivo studies. The strain of the mice used was C57BL/6 strain.

### 7.3 Mode of Administration

Exosomes were administered via intravenous mode (i.v) Groups: Group 1: Saline control; Group 2: UC-MSC-Exo (Naïve/primed).

### 7.4 Dosage Calculation

The doses of exosomes were determined based on an assessment of available clinical and preclinical data on the use of UC-MSCs and exosomes for therapeutic application.

### 7.5 Preclinical Study Dosage (Mouse Model)

Human to animal dose equivalence formula were calculated based on differences in body weight and surface area.
(i) Mouse dose (per kg body weight)=Human dose (per kg body weight)×12.3
(ii) Human dose: Exosomes were administered at a dose of 80-160 Billion (for an average body weight of 70 kg at 1.3-2.6 Billion/kg body weight).

Exosomes were administered at a high dose of 16-32 Billion exosomes/kg body weight.

### 7.6 LPS Dose (LPS Induced Lung Injury Model)

Given the body of work available, a dose between 10 mg and 100-125 mg was considered suitable in order to ensure that the study covers doses at sub-lethal and lethal concentrations. A dose of 100 mg was expected to induce mortality at 48-72 hours post LPS administration.

### 7.7 Bleomycin Dose (Bleomycin Induced Lung Fibrosis Model)

A single intra-tracheal dose of bleomycin (50 μL, 3 U/kg (2 mg/kg)) was determined.

### 7.8 In-Vivo Readouts:

Terminal readouts:
Survival rate
Histology: HandE. Masson's trichrome or Sirius Red
Assessment of total and differential blood cell count: Automated Analyzer
Fibrosis and inflammatory marker characterization
Inflammatory cytokine profiling in serum and BAL samples
Inflammatory cell type and sub-population analysis

Temporal readouts:
Assessment of total and differential blood cell count: Automated Analyzer
Inflammatory cytokine profiling in serum sample.

### Example 8

Generation of Therapeutically Enriched Exosomes from Primed BM-MSCS for Avascular Tissue (Cornea) Regeneration.

Based on the protein expression and correlation with higher regenerative potential v/s cargo proteins, or biomarkers, the highest scored exosomes were used for avascular tissue regeneration. The present example demonstrates the avascular tissue. i.e., cornea regeneration via enriched exosomes using macromolecule (CSSC-derived conditioned media (CSSC-CM) mediated priming of bone marrow derived mesenchymal stem cells. (hBM-MSCs).

8.1 Exosomes Derived from hBM-MSC Primed with CSSC-CM
8.1.1 CSSC Conditioned Media Induced Priming in hBM-MSCs

hBM-MSCs were cultured in xeno free media along with a replacement of 10% and 20% of CSSC-derived conditioned media (CSSC-CM) to reach a confluence of 80-90%. The media was then subsequently shifted to EV collect for 24-72 hours and primed conditioned media was collected (as described in example 1.1, and 1.2), and exosome isolation was performed by iodixanol density gradient method (as described in example 4.4) and enriched exosomes were obtained. Homogenous fraction F9 was considered for further functional analysis. The secretome analysis was performed using ELISA, detecting the levels of HGF, VEGF, sFLT1, IL-6, NGF.

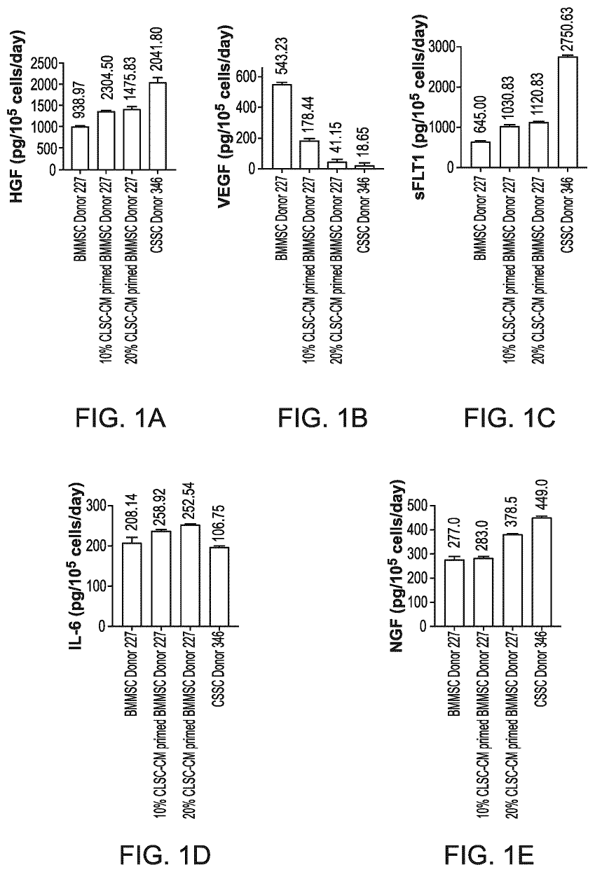

FIGS. 1A-IE shows the secretome profile of enriched exosomes derived from hBM-MSCs primed with CSSC-derived conditioned media (see example 3.2). FIG. 1A depicts a bar graph illustrated an increased level of secreted Hepatocyte growth factor (HGF) from exosomes derived from hBM-MSC primed with CSSC-CM compared to control exosomes. Similarly. FIGS. 1C-IE illustrate an increased level of secreted sFLT1, IL-6, and nerve growth factor (NGF) from exosomes derived from hBM-MSC primed with CSSC-CM compared to control exosomes, respectively. FIG. 1B demonstrates a significant reduced level of vascular endothelial growth factor (VEGF) from exosomes derived from hBM-MSCs primed with CSSC-CM compared to control exosomes. These results demonstrate that hBM-MSCs can be primed with CSSC-conditioned media to generate therapeutically enriched exosomes.

### 8.1.2 Characterization of Anti-Inflammatory Activity of Different CSSC-Conditioned Media Primed Exosome Variants

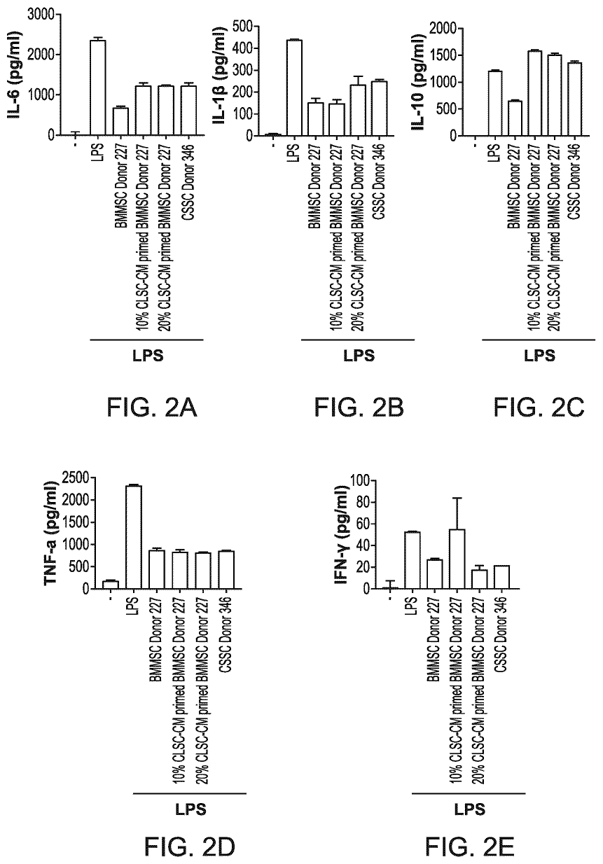

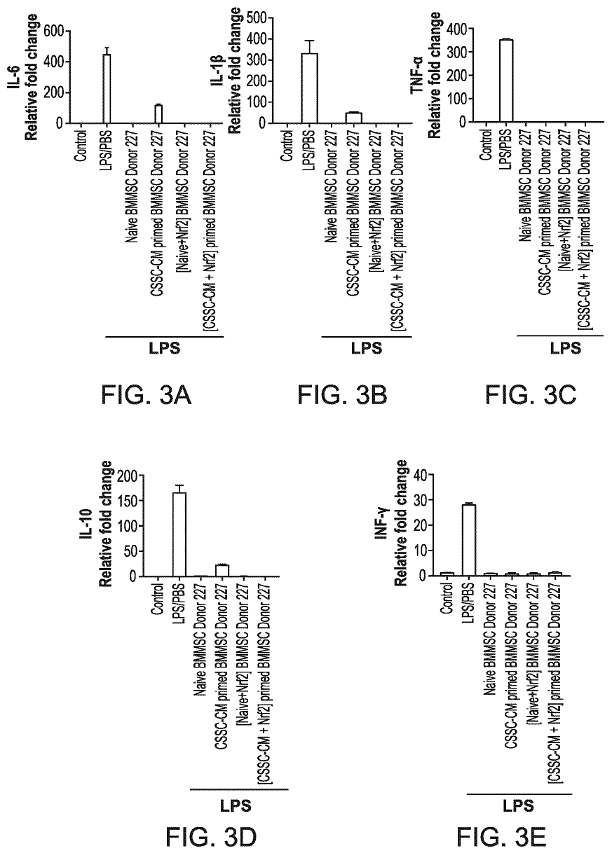

Different exosome variants derived from hBM-MSCs primed with CSSC-conditioned media (CSSC-CM) were tested to detect their anti-inflammatory activities using RAW 264.7 cells. RAW 264.7 cells were treated with lipopolysaccharide (LPS) binding protein to induce inflammation in presence of the exosomes to check the preventive/prophylactic effect of exosomes in inflammation. As illustrated in FIGS. 2A-2B, and 2D-2E, exosome variants derived from hBM-MSCs primed with CSSC-CM reduced inflammatory cytokine protein expression of key inflammatory cytokines (e.g., IL-6, IL-1β, TNF-α and IFN-γ) in RAW 264.7 cells treated with LPS. Furthermore, overall inflammatory cytokine gene expression was also reduced in RAW 264.7 cells stimulated with LPS and treated with exosome variants derived from hBM-MSCs primed with CSSC-CM. FIGS. 2A-3E demonstrate that exosome variants derived from hBM-MSCs and conditioned with CSSC media have anti-inflammatory activity by reducing inflammatory cytokine expression and inflammatory cytokine gene expression of key inflammatory cytokines (e.g., IL-6, IL-10, IL-1β, TNF-α and IFN-γ). Further, it can be inferred from FIGS. 3A-3E that the exosomes derived from hBM-MSCs primed with both the CSSC-CM and the Nrf2 activator DMF (see example 3.6) exhibit increased expression level of the anti-inflammatory cytokine IL-10 and reduced levels of IL-6, TNF α, IL-1β in RAW 264.7 cells treated with LPS.

8.1.3 Characterization of Anti-Fibrotic Properties of Different Exosome Variants Primed with CSSC Conditioned Media

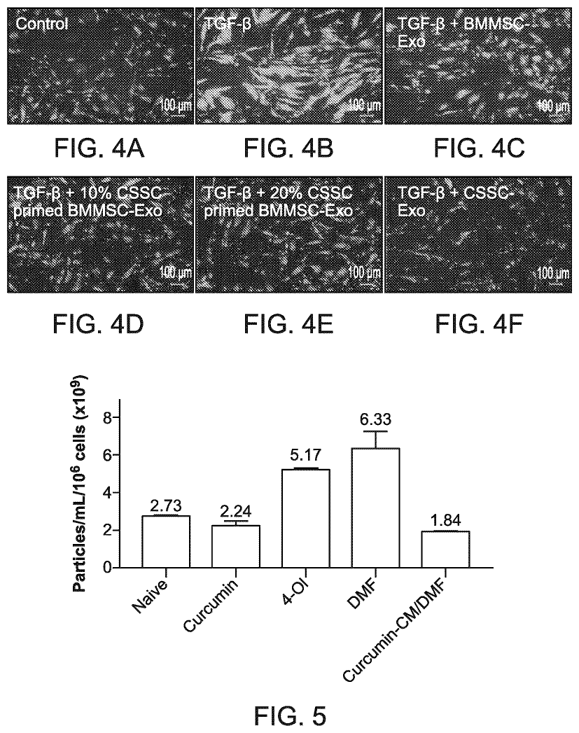

Different exosome variants derived from hBM-MSCs primed with CSSC-conditioned media (CSSC-CM) (see example 3.2) were tested to detect the anti-fibrotic activities of the primed exosomes. To test the anti-fibrotic activities of the exosome variants, human dermal fibroblast cells were treated for 24 hours with TGF-β (FIG. 4B) or co-treated with TGF-β and indicated exosomes (FIGS. 4C-4F) to induce fibrosis (similar to example 6.3). α-SMA expression was monitored as a fibrotic marker to check the efficacy of the exosome variants. Referring to FIGS. 4D-4E, it can be observed that exosomes derived from hBM-MSCs primed with 20% and 10% CSSC-CM were able to inhibit TGF-β induced α-SMA expression in the fibroblast cells. Cells treated with naïve exosomes derived from hBM-MSCs expressed α-SMA to a lesser extent (FIG. 4C) compared to primed exosomes (FIGS. 4D-4E). Exosomes derived from hBM-MSCs primed with 20% and 10% CSSC-CM were able to efficiently inhibit fibrosis in fibroblasts compared to control and naïve exosome treatment.

8.2 Exosomes Derived from hBM-MSC Primed with Nrf2 Activator (DMF or 4-OI)
8.2.1 NRF2 Activator (DMF OR 4-OI) Mediated Priming of hBM-MSC Cells and Characterization of Secretome and Exosomes Profile

hBM-MSC was cultured in xeno-free media recommended by the manufacturer, hBM-MSC cells were grown to reach confluency of (80-90) % and treated with a Nrf2 activator (DMF-100 μM) for 24 hours before shifting and maintaining the cells in EV collect media for 24-72 hours (see example 3.3). After 72 hours, condition media was collected, and exosome isolation was performed using iodixanol density gradient protocol (as described in example 4.4).

FIG. 5 illustrates a bar graph quantifying the yield of naïve exosomes, exosomes derived from hBM-MSCs primed with various Nrf2 activators (e.g., DMF or 4-OI) and exosomes derived from hBM-MSCS primed with other priming agents such as curcumin. It can be observed from FIG. 5 that exosomes derived from hBM-MSc and primed with the Nrf2 activator DMF or 4-OI each produced a higher yield of exosomes compared to untreated (naïve) exosomes and exosomes primed with other priming agents (e.g., curcumin or the combination of curcumin and DMF). Alternatively. FIG. 5 also suggests that Nrf2 activators have no inhibitory effect in exosome secretion of the cells, making them a good priming agent.

### 8.2.2 Secretome Marker Profiling of Primed Cells Using Priming Agents

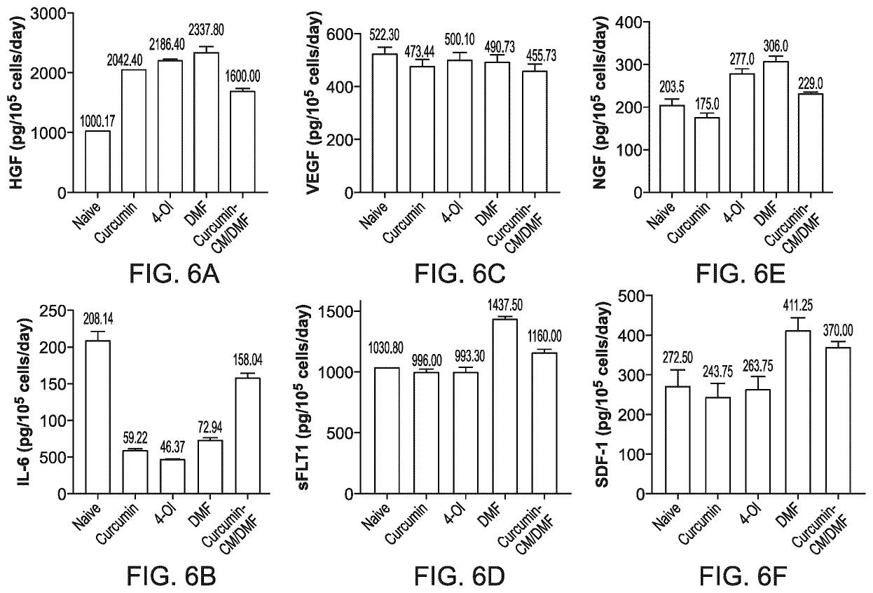

hBM-MSCs were primed with various priming agents (small molecules) and the secretome was collected (see example 3.2-3.6). The secretome analysis was performed using ELISA, and the levels of HGF, VEGF, NGF, IL-6, sFLT1, SDF1 were detected. FIGS. 6A-6F depict bar graphs of the quantification of the secreted protein levels of HGF, VEGF, NGF, IL-6, sFLT1, and SDF-1 from hBM-MSCs primed with curcumin, 4-OI, DMF, or the combination of curcumin conditioned media and DMF.

The Nrf2 activators, 4-OI and DMF, each generated a significant increase in HGF secretion as compared to other primed variants and the untreated control (FIG. 6A). VEGF secretion levels were unaltered, regardless of the priming agent used (FIG. 6C). Priming with DMF did lead to an increase in secretion of sFLT1 (FIG. 6D). NGF (FIG. 6E), and SDF (FIG. 6F). Further, referring to FIG. 6B, it can be observed that curcumin and the Nrf2 activators (4-OI and DMF) each attenuated the secretion of IL-6. It is pertinent to note that NGF secretory levels were enhanced by each of the Nrf2 activators (4-OI and DMF) (FIG. 6E). It was also observed from FIGS. 6A-6F that the combination of curcumin-conditioned media+DMF did not appear to have a pronounced effect on the levels of HGF, VEGF, NGF, IL-6, sFLT1, SDF1.

8.2.3. Profiling of Exosome Cargo from Exosomes Derived from Cells Primed with Various Priming Agents.

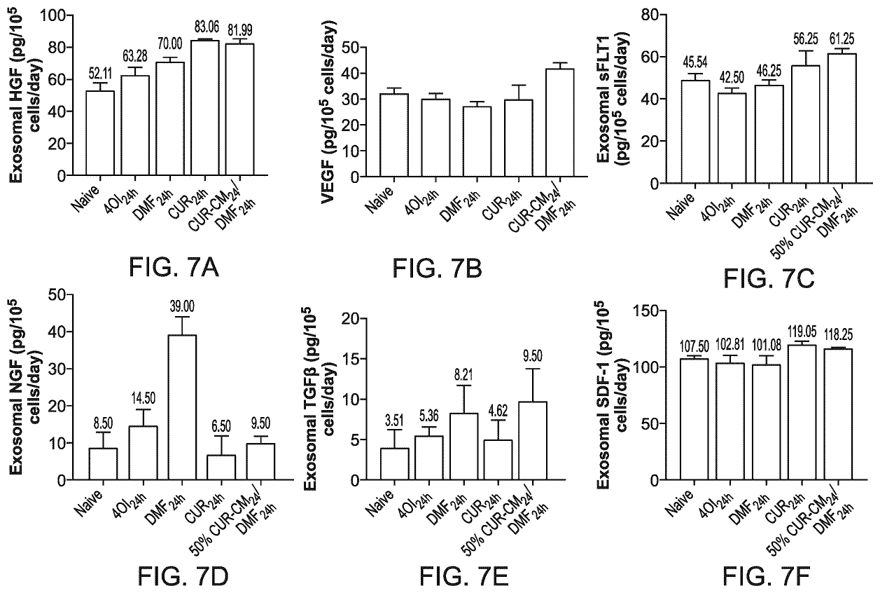

Reference is made to FIGS. 7A-7F, hBM-MSCs were primed with the indicated priming agents and secretome was collected and exosome isolation was performed using Pandorum's optimized iodixanol density gradient method (see example 4.3) and the levels of HGF (FIG. 7A), VEGF (FIG. 7B), sFLT1 (FIG. 7C), NGF (FIG. 7D). TGF-β (FIG. 7E, and SDF1 (FIG. 7F) were detected in purified fraction 9 using ELISA. As illustrated by FIG. 7D, exosomes derived from DMF primed hBM-MSCs contain significantly higher levels of exosomal NGF as compared to naïve exosomes or exosomes derived from hBM-MSCs primed with other priming agents, such as 4-OI, curcumin (CUR), or combined curcumin and DMF (CUR/DMF). Likewise, priming hBM-MSCs with DMF led to an increase in exosomal TGF-p compared to naïve exosomes, curcumin primed, or 4-OI primed exosomes.

8.2.4. Anti-Inflammatory Activity of Exosomes Derived from Primed BM-MSCs

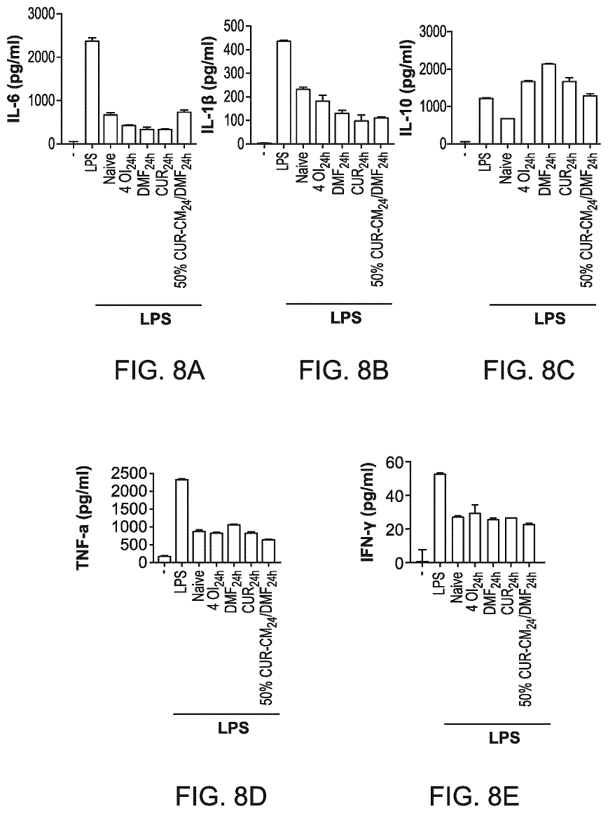

The exosome variants derived from hBM-MSC primed with the Nrf2 activator DMF (see example 6.2) were tested to detect their anti-inflammatory activity using RAW 264.7 cells. RAW 264.7 cells were treated with LPS to induce inflammation in presence and absence of exosomes derived from primed hBM-MSCs and key inflammatory cytokines were measured by ELISA to determine the preventive effect of exosomes in inflammation. As shown in FIGS. 8A-8E, exosomes derived from hBM-MSC primed with DMF or curcumin demonstrated a reduction in IL-6, IL-1β, TNF-α and IFN-γ expression at the protein level. Additionally, the anti-inflammatory cytokine IL-10 was increased significantly in exosomes derived from hBM-MSC primed with DMF or curcumin, indicating that not only does DMF and curcumin primed exosomes reduce common inflammatory cytokines but also have anti-inflammatory effects as well.

8.3 Exosomes Derived from hBM-MSCs Primed with CSSC-CM and a Nrf2 Activator
8.3.1 Profiling of Exosome Cargo from Exosomes Derived from hBM-MSCs Primed with CSSC-conditioned media, the Nrf2 activator or both.

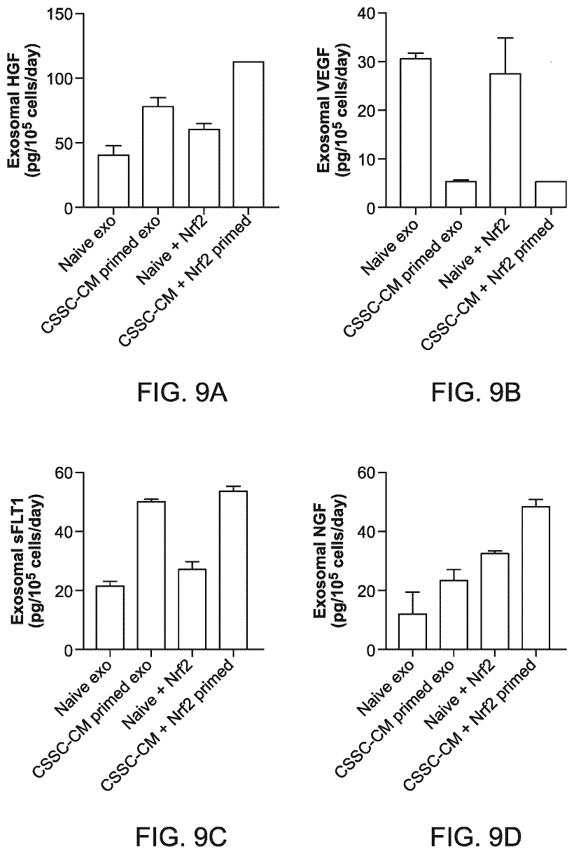

hBM-MSC was cultured in xeno free media recommended by the manufacturer, hBM-MSCs were grown in presence of CSSC-CM at a concentration of 20% total media volume until the cells reached 80-90% confluency. The media was then change to have the hBM-MSCs treated with a Nrf2 activator (DMF-100 μM) for 24 hours before shifting the cells in EV collect media and maintaining in EV collect media for 72 hours. After 72 hours, the conditioned media was collected, and exosome isolation (purification) was performed using iodixanol density gradient protocol (as described in example 4.4). The levels of HGF, VEGF, sFLT1, and NGF were detected in different combinatorial primed exosome variants using the ELISA (FIGS. 9A-9D). Although singularly primed (Nrf2 activator or CSSC-conditioned media) exosomes demonstrated an increase in exosomal HGF, sFLT1 and NGF, the combinatorial primed (CSSC-CM and DMF) exosomes had the highest levels of exosomal HGF, sFLT1, and NGF.

Both CSSC-CM primed exosomes and DMF primed exosomes demonstrated an increase in exosomal HGF. The expression level of exosomal HGF in DMF-primed exosomes was about 1.2 times (1.2×) that of naïve exosomes and The expression level of exosomal HGF in CSSC-CM-primed exosomes was about 2 times (2×) that of naïve exosomes. That being said, combinatorial primed (CSSC-CM+DMF) showed the highest increase in exosomal HGF, more than twice (2×) or about three times (3×) the expression level of exosomal HGF compared to naïve exosomes, and approximately twice the expression level of exosomal HGF compared to DMF-only priming (FIG. 9A). In addition, CSSC-CM primed exosomes and combinatorically primed (CSSC-CM+DMF) exosomes demonstrated both had substantially reduced exosomal VEGF expression, less than a quarter (1/4 or 25%) of VEGF expression, compared to naïve exosomes or DMF primed exosomes (FIG. 9B).

Singularly primed (CSSC-CM or DMF) exosomes demonstrated increased exosomal sFLT1 and NGF (FIGS. 9C-9D). The expression level of exosomal NGF in DMF-primed exosomes was more than 2 times (2×) or about 3 times (3×) that of naïve exosomes and the expression level of exosomal NGF in CSSC-CM-primed exosomes was about 2 times (2×) that of naïve exosomes. However, combinatorial primed (CSSC-CM and DMF) exosomes had the highest exosomal sFLT1 and NGF, indicating that combinatorial priming with CSSC-CM and DMF may lead to a more desired exosome cargo compared to singular priming of exosomes or naïve exosomes. The exosomal sFLT expression level of combinatorial primed (CSSC-CM and DMF) exosomes was more than twice that of naïve exosomes and about twice that of DMF primed exosomes. The exosomal NGF expression level of combinatorial primed (CSSC-CM and DMF) exosomes was more than three times (3×) that of naïve exosomes and about 1.5 times (1.5×) that of DMF primed exosomes.

8.3.2 Characterization of Anti-Inflammatory Activity of Different Exosome Variants Primed with CSSC Condition Media and with NRF2 Activator (DMF)

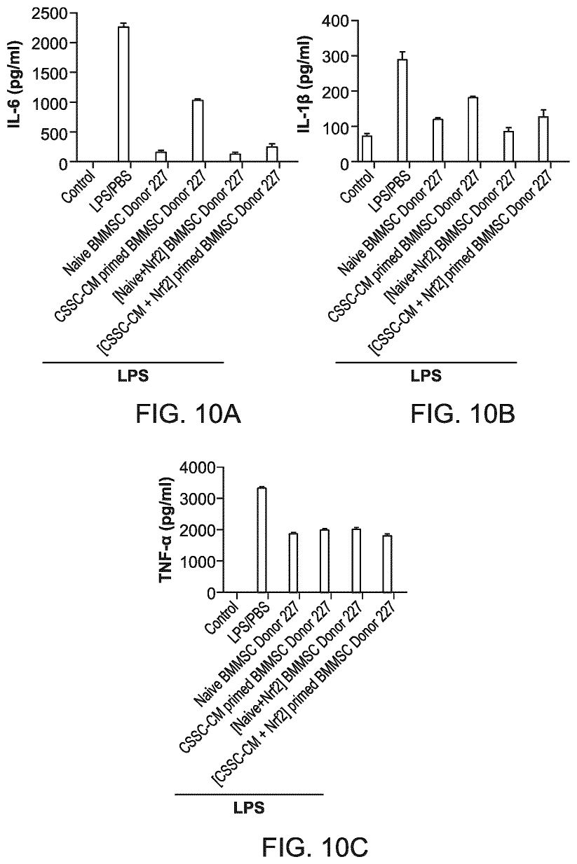

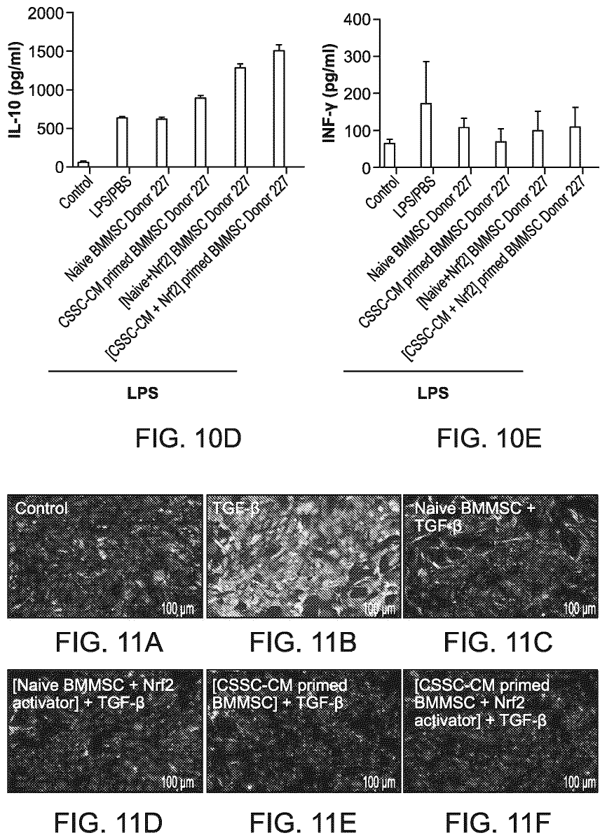

Exosomes derived from hBM-MSCs primed with either CSSC-condition media, the Nrf2 activator DMF, or the combination thereof were tested using RAW 264.7 cells treated with LPS to detect anti-inflammatory activity of the primed exosomes. Inflammatory and anti-inflammatory cytokines were measured by ELISA. As demonstrated in FIGS. 10A-10E, exosomes derived from hBM-MSCs combinatorically primed with CSSC-CM and the Nrf2 activator (DMF) were able to reduce inflammation by reducing IL-6 (FIG. 10A), IL-1β (FIG. 10B), TNF-α (FIG. 10C), and IFN-γ (FIG. 10E) expression at protein level. Additionally, the expression of the anti-inflammatory cytokine IL-10 was increased with the treatment of singularly primed exosomes (CSSC-CM or DMF). Moreover, the highest increase in IL-10 expression was achieved with exosomes derived from the the combinatorial priming of hBM-MSCs with CSSC-CM and DMF.

These data indicate that exosomes derived from hBM-MSCs primed with both CSSC-CM and DMF effectively reduced expression of inflammatory cytokines while increasing expression of anti-inflammatory cytokines.

8.3.3 Characterization of Anti-Fibrotic Activity of Exosomes Derived from Cells Combinatorically Primed with CSSC Condition Media+NRF2 Activator (DMF)

Exosomes derived from hBM-MSCs primed with CSSC-CM and DMF were tested to detect their anti-fibrotic activities. Human dermal fibroblast cells were treated with TGF-β to induce fibrosis (see example 6.3). The fibroblast cells were treated with primed exosomes to test the exosomes' anti-fibrotic activity. α-SMA expression, a fibrotic marker, was monitored using immunofluorescence to check the efficacy of the exosomes' anti-fibrotic activity, after treatment. Representative immunofluorescence images as illustrated in FIGS. 11A-11F show that even singularly primed exosomes (CSSC-CM or DMF) reduced fibrosis (FIGS. 11D and 11E). However, exosomes derived from hBM-MSCs primed with the combination of CSSC-CM and DMF demonstrated the most reduction in fibrosis (FIG. 111F).

8.3.4 Characterization of the wound healing activity of exosomes derived from hBM-MSCs primed with CSSC condition media, the Nrf2 activator (DMF), or the combination thereof.

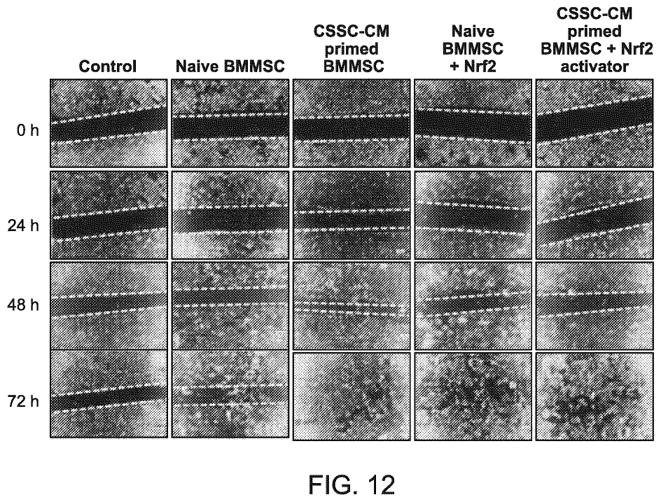

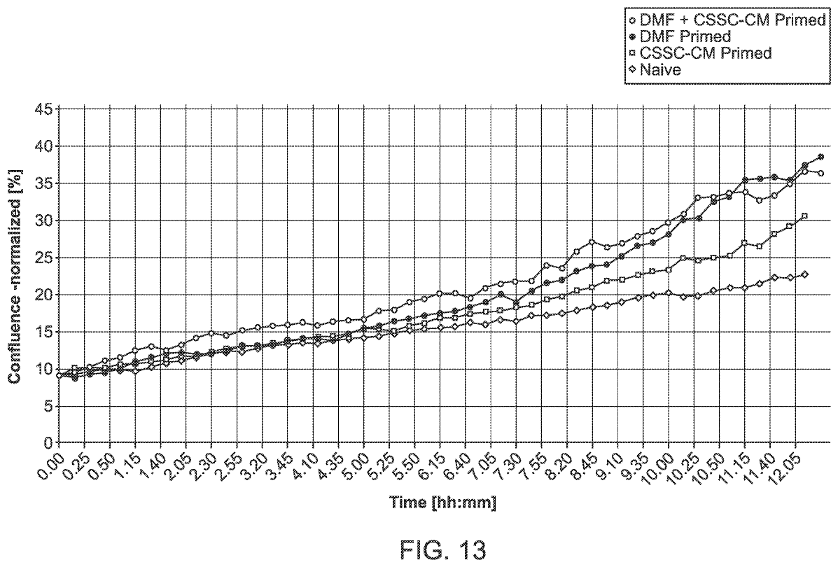

Reference is made to FIG. 12. Exosomes derived from hBM-MSCs primed with CSSC-CM, the Nrf2 activator (DMF) or the combination thereof were tested to detect their effect in a 2D scratch assay (see example 6.2). Immortalized human corneal epithelial cells (hTCEPi) were labeled with a green fluorescence dye (CMFDA), and a scratch was generated through the hTCEPi cells. The hTCEPi cells were treated with naïve exosomes (Naïve BM-MSCs), singularly primed exosomes (CSSC-CM or DMF) or the combination thereof and wound closure was observed at the following time points 0, 24 hours, 48 hours, and 72 hours using fluorescence microscope. Singularly primed exosome treatment and the combination thereof led to wound closure by 72 hours, indicating that primed exosome treatment led to significant directional cell migration. Similarly, a quantitative assay of corneal cell migration and proliferation after exosome treated was performed, as indicated in FIG. 13. Corneal cells treated with exosomes derived from hBM-MSCs primed with CSSC-CM and DMF had the highest cell proliferation throughout almost all time points, while corneal cells treated with DMF primed exosomes had increased cell proliferation at later time points compared to corneal cells treated with CSSC-CM primed exosomes or naïve exosomes alone.

8.3.5 Characterization of the Wound Healing Activity in Rabbit Corneas of Exosomes Derived from hBM-MSCs Primed with CSSC-CM and DMF.

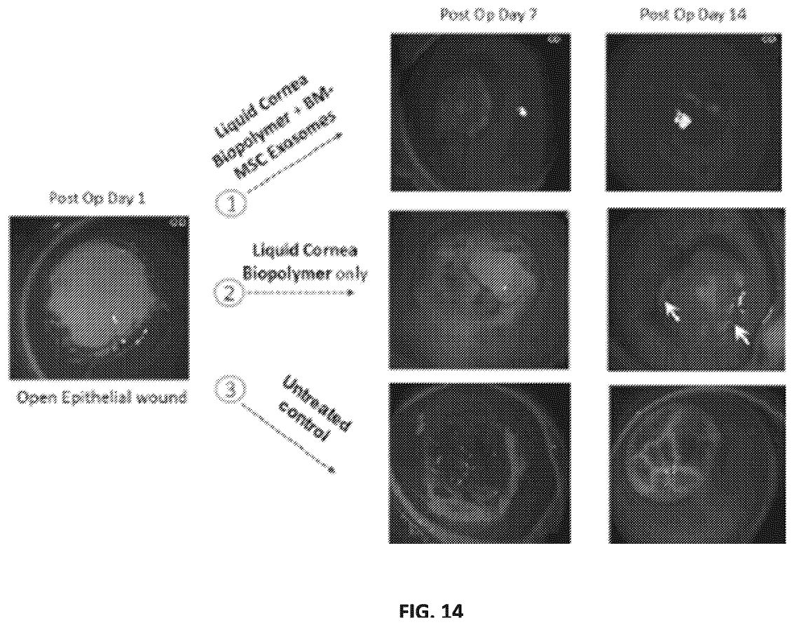

FIG. 14 depicts representative microscopic images of rabbit corneas having open epithelial wounds after day 1, day 7, and day 14 after surgery. To test for the ability of primed exosomes derived from hBM-MSCs to effectively induce wound healing in a 3D model, injured rabbit corneas were treated with a liquid cornea biopolymer in combination with exosomes derived from hBM-MSCs primed with CSSC-CM and the Nrf2 activator DMF, a liquid cornea biopolymer alone, or an untreated control. At day 7 post operation, the combination of the liquid cornea biopolymer and the exosomes derived from hBM-MSCs primed with CSSC-CM and the Nrf2 activator DMF demonstrated significant wound healing compared to liquid cornea biopolymer alone or the untreated control. At day 14, the combination of the liquid cornea biopolymer and the exosomes derived from hBM-MSCs primed with CSSC-CM and the Nrf2 activator DMF demonstrated complete and stable epithelialization of the rabbit cornea. Treatment with liquid cornea biopolymer alone demonstrated near complete epithelization with some patches of breakdown at Day 14 while the untreated control demonstrated defective epithelization. Taking FIGS. 12, 13, and 14 together, exosomes derived from hBM-MSCs combinatorically primed with both CSSC-CM and DMF demonstrated superior wound healing activity based on a 2D scratch assay and corneal cell migration and proliferation analysis, and demonstrated robust wound healing in rabbit corneas in vivo.

### Example 9

Generation of Therapeutically Enriched Exosomes from Primed UC-MSCs/WJ-MSCs for Vascular Multi Tissue Regeneration (Liver, Lung)

The present example demonstrates an in-vitro culture of umbilical cord blood derived mesenchymal stem cells (UC-MSCs)/Wharton Jelly derived MSCs (WJ-MSCs). The protocol for culturing UC-MSCs and WJ-MSCs is described in example 1.5. Further, the unique sub-population of stem cells (UC-MSCs and WJ-MSCs) was selected based on the method described in example 1.4. The expanded population of stem cells were then obtained and primed with different priming agents alone or in combination thereof: (a) Nrf2 activators, such as DMF or 4-OI, or CDDO-Im; and (b), SRT1 activators: SRT-2104, or Resveratol. The priming of stem cells can be in absence or presence of hypoxia. Priming of stem cells with the priming agents as mentioned here, allows for providing or obtaining the primed stem cells and primed conditioned medium with enhanced regenerative, stemness and anti-inflammatory properties. The MSCs primed with a single priming agent, such as Nrf 2 activator or SIRT1 activator, or with combination of priming agent, such as Nrf2 activator +SIRT1 activator were used as a source for different exosome variant production with specific/enriched cargo loaded factors. Exosome variants were then characterized both at the physical and molecular level for their functional efficacy. Characterized exosome variants were categorized based on their functionality for different inflammatory and fibrosis associated diseases such as pulmonary dysfunction, acute respiratory distress, inflammation associated disorders including but not limited to rheumatoid arthritis, systemic juvenile idiopathic arthritis, idiopathic pulmonary fibrosis, acute respiratory distress syndrome (ARDS), pneumonia, bronchitis, chronic obstructive pulmonary disease (COPD), COVID-19, coronavirus class of infection, cystic fibrosis, hantavirus, influenza, tuberculosis, systemic lupus, osteoarthritis, NASH, liver fibrosis. Mooren's ulcer, neurotrophic ulcer, myocardial infarction, etc.

Overall, the present disclosure provides a method of providing or obtaining primed stem cells, and a primed conditioned medium. The method involves the step of isolating a population of mesenchymal stem cells, such as UC-MSCs, and WJ-MSCs expressing signature set of markers. The selected population of MSCs were then modified using hTERT (human telomerase reverse transcriptase), wherein hTERT extends the doubling potential of MSCs, which helps in facilitating scalable and homogenous population of cells. The stem cells are further cultured in 3D culture system (microcarrier-based system, or spheroid-based system, or hollow-fiber bioreactors), to obtain expanded population of stem cells. The expanded population of stem cells are further primed with different priming agents (small molecules and macromolecules). The presence of priming agents (small molecules, and macromolecules) along with the disclosed concentration range, and the duration of treatment of the priming agents on cells may be critical for providing or obtaining the primed stem cells and primed conditioned medium. Priming of naïve stem cells using different priming agents used alone or in combinations thereof, helps in enhancing regenerative, stemness and anti-inflammatory properties of the cells. The primed stem cells are further used as a source of different exosome variants that are enriched with anti-inflammatory factors, anti-fibrotic factors, pro-angiogenic factors. The enriched therapeutic grade exosomes are then further applied for vascular tissue regeneration (lung or liver), or for avascular tissue regeneration (cornea). With the disclosed method of the present disclosure, the high yield of enriched primed stem cells, or primed exosomes, a large number of patients suffering from diseases including, but not limited to, rheumatoid arthritis, systemic juvenile idiopathic arthritis, idiopathic pulmonary fibrosis, acute respiratory distress syndrome (ARDS), acute lung injury (ALI), pneumonia, bronchitis, chronic obstructive pulmonary disease (COPD), COVID-19, coronavirus class of infection, cystic fibrosis, hantavirus, influenza, tuberculosis, systemic lupus, myocardial infarction, osteoarthritis, non-alcoholic fatty liver disease (NASH), liver fibrosis, Mooren's ulcer, neurotrophic ulcer, and corneal keratitis (CK), dry eye disease ulcer, herpetic simplex keratitis, post-LASIK ectasia, postoperative corneal melts, post keratoprosthesis melts, corneal perforations, neurotrophic keratitis (NK), keratoconus Sjögren's syndrome, mucous membrane pemphigoid, Stevens-Johnson syndrome, chemical burns, and thermal burns, can be treated.

### Example 10

Treatment of Non-Alcoholic Steatohepatitis (Nash) Induced Liver Spheroids with Primed Exosomes

The present example demonstrates an in-vitro treatment of an induced non-alcoholic steatohepatitis (NASH) phenotype in human liver spheroids using exosomes derived from naïve hBM-MSCs grown under standard conditions (naïve exosomes) or from hBM-MSCs primed with DMF (primed exosomes). The protocol for culturing and priming hBM-MSCs, as well as acquiring exosomes from the primed hBM-MSCs, are described in Examples 3 and 4, as well as Example 8. In particular, the protocol for producing the DMF-primed MSC-derived exosomes used in treating the NASH-induced liver spheroids in the present Example is described in section 8.2.1 of Example 8.

Non-alcoholic fatty liver disease (NAFLD) is a condition in which fat builds up in a subject's liver, unrelated to the subject's alcohol consumption. The disease comes in several stages. Where the liver has excess fat has not developed inflammation or fibrosis, then the disease may be referred to as liver steatosis or non-alcoholic fatty liver (NAFL). Once the disease progresses further and the liver tissue develops inflammation and fibrosis, the disease is referred to as non-alcoholic steatohepatitis (NASH).

The protocol for generating human liver spheroids and inducing a NASH phenotype in the human liver spheroids for the present study was as follows: A mix of primary hepatocytes and liver stellate cells from human donors were co-cultured at a 70:30 ratio (70 hepatocytes to 30 liver stellate cells). The cell mixture was seeded on ultra-low attachment plates and cultured to form viable liver spheroids. Once viable liver spheroids were obtained, a NASH phenotype was induced in the liver spheroids through a sequential treatment of free fatty acids to induce steatosis followed by a treatment of TGF-β to induce fibrosis, so that the liver spheroids would demonstrate aspects of a steatofibrotic liver. The NASH induction protocol was as follows: On day 7 post seeding, the spheroids were treated with 600 μM free fatty acid (FFA) mixture consisting of Oleic acid and Palmitic acid at a weight ratio of 2:1 for 6 days with media change every other day. After 6 days of FFA treatment (13 days post seeding), 20 ng/mL of TGF β1 was added to the FFA-containing media for a further two days of combined FFA and TGFβ1 treatment. At day 15 post seeding (after 8 days of FFA treatment and 2 days of combined FFA and TGFβ1 treatment), the media was changed to TGF β1-only treatment, with 20 ng/mL TGF β1 and without the FFA mix. Two days later, at day 17 post seeding, the spheroids were designated as NASH-induced (that is, exhibiting aspects of steatofibrosis) and subject to therapeutic treatment (for example with naïve or primed exosomes. HGF, or vehicle control), without FFA or TGFβ1. Two days later, at day 19 post seeding, the spheroids and the conditioned media were harvested for characterization. The above NASH-induction protocol may be summarized as follows:
Day 0: seeding of primary liver cells
Day 7: treated with 600 μM FFA mixture (2:1 mixture of Oleic acid and Palmitic acid)
Day 13: TGFβ1 added for combined FFA and TGFβ1 treatment
Day 15: TGFβ1—only treatment
Day 17: therapeutic treatment (naïve or primed exosomes. HGF, or vehicle control)
Day 19: harvesting for analysis.

Following NASH induction with the sequential treatment with the free fatty acid mixture followed by TGF-β, liver spheroids demonstrated a NASH-like phenotype including a reduced spheroid size, increased collagen deposition, and a decrease in albumin secretion. The NASH-induced liver spheroids were then subjected to administration of exosomes from the naïve or primed hBM-MSCs and used in further assays as explained herein below.

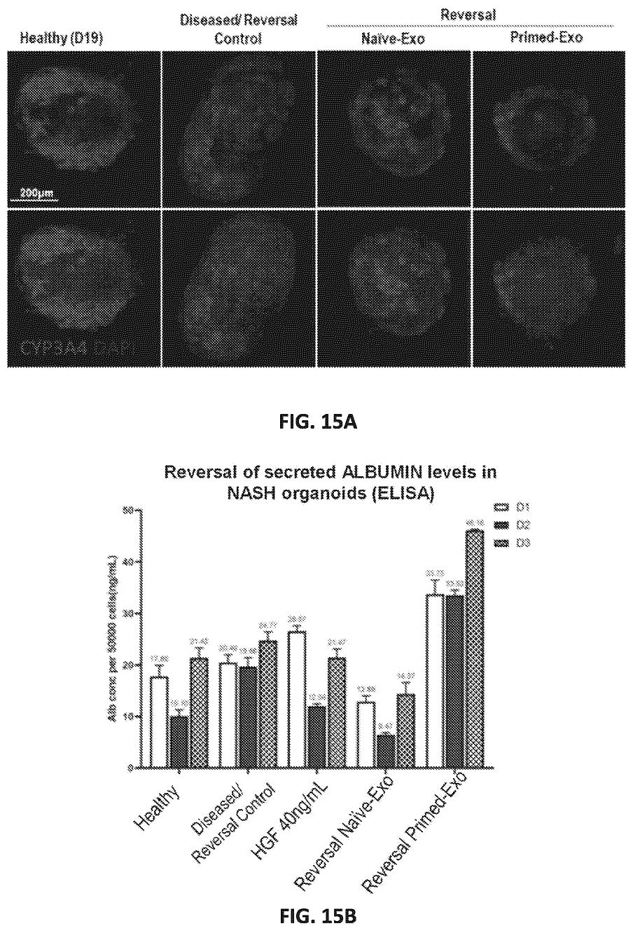

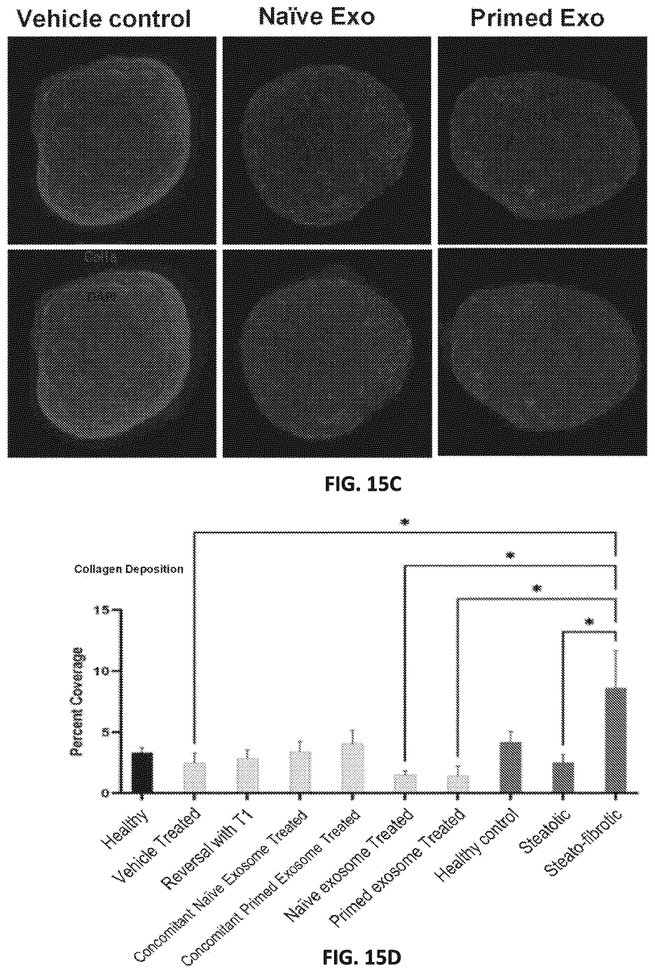

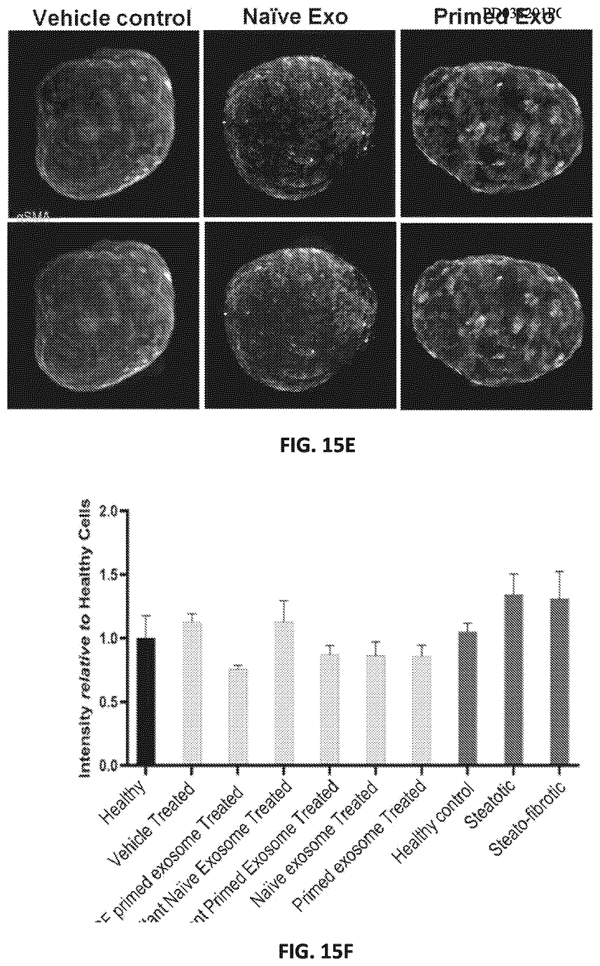

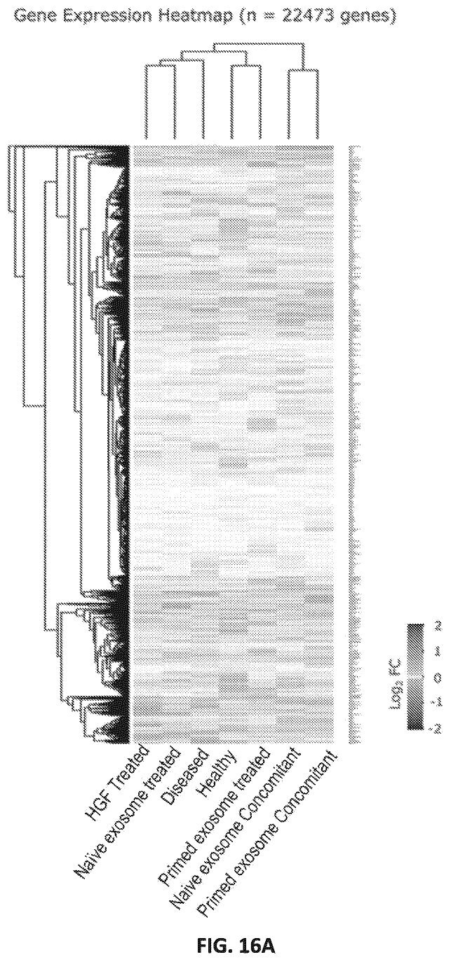

FIG. 15A, depict representative immunofluorescence images stained for CYP3A4 and DAPI in liver spheroids under four conditions: (1) healthy (without NASH induction): (2), NASH-induced (“Diseased/Reversal control”); (3) NASH-induced followed by treatment with naïve exosomes (“Naïve-Exo”); and (4) NASH-induced followed by treatment with primed exosomes (“Primed Exo”). DAPI is a nucleus maker and CYP3A4 is a marker used to indicate healthy liver tissue, so that a reduction in CYP3A4 staining indicates reduced liver tissue health and an increase in CYP3A4 staining indicates improved liver tissue health. Liver spheroids were imaged similar to as described in example 6.3.

It was found that NASH-induced liver spheroids exhibited a reduction in CYP3A4 staining compared to healthy liver spheroids not subjected to NASH induction, and treatment of the NASH-induced liver spheroids treated with primed exosomes (“Primed-Exo”) from DMF-primed hBM-MSCs exhibited a partial restoration of the CYP3A4 staining indicating that the exosome treatment was effective in at least partially restoring the health of the NASH-induced spheroids. By contrast, treatment with the naïve exosomes (“Naïve exo”) did not exhibit restored CYp3A4 staining, compared with naïve exosome treatment.

Secreted albumin levels is another marker of liver health. FIG. 15B illustrates a bar graph showing albumin levels secreted in to the culture medium by the liver spheroids under difference conditions, including: (1) healthy at D19 (without NASH induction); (2), NASH-induced at D19 (“Diseased/Reversal control”); (3) NASH-induced followed by treatment with 40 ng/ml hepatic growth horomone at D17-D19 (HGF”); (4) NASH-induced followed by treatment with naïve exosomes at D17-D19 (“Naïve-Exo”); and (4) NASH-induced followed by treatment with DMF-primed exosomes at D17-D19 (“Primed Exo”). The secreted albumin in the culture medium was quantified based on ELISA measurements. Measurement were taken from respective culture media samples at 24 hours, 48 hours, and 72 hours after treatment (or equivalent timeframe for the healthy and NASH-induced conditions). It was found that NASH-induced liver spheroids treated with DMF-primed exosomes exhibited upregulated albumin secretion, indication improved spheroid health. By contrast, treatment of NASH-induced liver spheroid with HGF or with naïve exosomes did not result in increased albumin secretion by the spheroids.

Collagen may serve as a marker for fibrosis in liver tissue, including liver spheroids. FIG. 15C, depict representative immunofluorescence images stained for collagen 1 in liver spheroids under three conditions: (1). NASH-induced and treated with vehicle control at D17-D19 (“Vehicle control”); (2) NASH-induced followed by treatment with naïve exosomes at D17-D19; and (3) NASH-induced followed by treatment with DMF-primed exosomes at D17-D19 (“Primed Exo”). Liver spheroids were imaged similar to as described in example 6.3. Treatment of NASH-induced liver spheroids with the primed exosomes reduced cell surface collagen protein expression compared treatment with vehicle control or naïve exosomes.

FIG. 15D illustrates a bar graph of the quantification of percent coverage of collagen deposition in liver spheroids under the following conditions:

Healthy D19 serve as healthy control for the reversal groupd, Vehicle treated (Diseased-D19), whereas the healthy control on the right side corresponds D17 disease induction group, serve as a control for FFA and FFA+TGFb1 treatment
(1) prior to NASH induction at D19 (“healthy”);
(2) NASH-induced and treated with vehicle control at D17-D19, harvested at D19 (“Vehicle Treated”);
(3) HGF treatment at D17-D19, harvested at D19 “Reversal with T1”;
(4) concomitant treatment with naïve exosomes during fibrosis induction with TGFβ1 at D13-D17 (“Concomintant Naïve Exosome Treated”);
(5) concomitant treatment with primed exosomes during fibrosis induction with TGFβ1 at D13-D17 (“Concomintant Primed Exosome Treated”);
(6) treatment with naïve exosomes at D17-D19 after NASH induction (“Naïve Exosome Treatment”);
(7) treatment with primed exosomes at D17-D19 after NASH induction (“Primed-Exosome Treatment”);
(8) not subjected to NASH induction, harvested at D17 (“Healthy Control”);
(9) Steatosis induction—treated with FFA mix only without TGFBI and harvested at D15;
(10) Steatofibrosis induction with sequence treatment with FFA and TGFβ1, harvested at D19 (see FIG. 15G; “Streato-fibrotic”.

The comparison of “healthy control” with “Steatofibrotic” shows that induction of NASH with FFA and TGFβ1, but not induction of steatosis only with FFA treatment, induced fibrosis in the liver spheroid. Based on collagen 1 staining alone, addition of either naïve or primed exosomes appeared to have prevented TGFβ1-induced fibrosis. Also based on the collagen 1 staining, it appears that cessation of TGFβ1 treatment and subsequent treatment with vehicle for two days was sufficient to reverse the TGFβ1-induced fibrosis. However, it was found that treatment of the NASH-induced liver spheroids with naïve exosomes and primed exosomes were both more effective than vehicle in reducing fibrosis compared to vehicle treatment only.

FIG. 15E depict representative immunofluorescences images of NASH-induced liver spheroids including exosome treated NASH-induced liver spheroids stained for α-SMA, another fibrosis marker, using the same set of conditions for liver spheroids as shown with respect to collagen staining in FIG. 15C. FIG. 15F illustrates a bar graph of relative intensity of α-SMA positive cells compared to healthy cells, using the same set of conditions for liver spheroids as shown with respect to collagen staining in FIG. 15D. Based on α-SMA staining alone, it appears that NASH induction with the FFA mixture and the TGFβ1, as well as steatosis induction with FFA mixture only, both induce fibrosis. Also based on α-SMA staining alone, it appears that both naïve and primed exosomes were more effective than vehicle in reducing the NASH-induced fibrosis. Also based on α-SMA staining alone, it appears that concomitant treatment with the primed exosomes, but not the naïve exosomes, prevented the NASH induction-based increase in α-SMA.

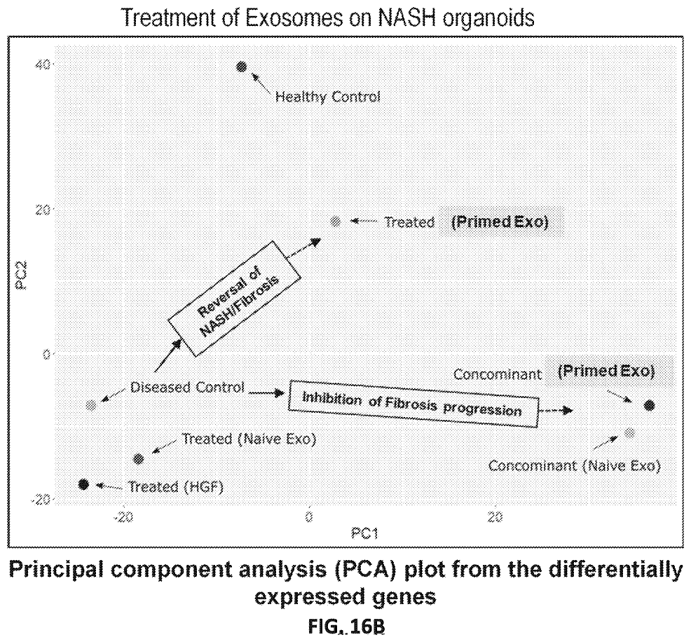

As described herein above and shown in FIGS. 15A-15F, the effect on the liver spheroids of treatment with disease-state induction agents such the FFA mixture or TGFβ1 and/or with therapeutic agents such as the exosomes can be assayed one marker (or a new markers) at a time with techniques such as immune-staining and ELISA. However, a much larger count (dozens, hundreds, thousands) of markers may be assay simultaneously using high-throughput techniques, such as microarray or next generation sequencing (NGS). FIG. 16A depicts a heatmap of changes in gene expression of liver spheroids based on microarray hybridization data. Microarray hybridization data was acquired as follows: After growth of liver spheroid samples and treatment with NASH-induction agents and/or with therapeutic agents as describe above, the liver spheroids were isolated, lysed, and mRNA was isolated, then converted to sequences of complementary DNA (cDNA). The cDNA samples were then applied to an Agilent® microarray and read with a microarray reader. The microarray analysis was performed for a predetermined transcriptome set of 22,473 genes (included in the microarray) using standard methods to obtain normalized expression values. The microarray analysis was performed with liver spheroid samples under the following conditions (“liver spheroid states”):
(1) treatment with 40 ng/ml HGF at D17-D19 after NASH induction (“HGF”);
(2) treatment with naïve exosomes at D17-D19 after NASH induction (“Naïve exosome treated”);
(3) NASH-induced, at D19 (“Diseased”);
(4) not subjected to NASH induction, at D19 (“Healthy”);
(5) treatment with primed exosomes at D17-D19 after NASH induction (“Primed exosome treated”);
(6) concomitant treatment with naïve exosomes during fibrosis induction with TGFβ1 at D13-D17 (“Naïve exosome Concomitant”); and
(7) concomitant treatment with primed exosomes during fibrosis induction with TGFβ1 at D13-D17 (“Primed exosome Concomitant”).

Each condition was based on a pool of 30 spheroids, and the heatmap was generated based on expression profile data from the pooled samples.

In the heatmap, each row represents a gene, and each column represents a liver spheroid state. The columns are arranged based on a hierarchical clustering analysis of the gene expression patterns in each of liver spheroid states. Pairs of liver spheroid states that cluster in the vicinity of each other with respect to their respective gene expression patterns are arranged as adjacent columns, and the brackets connecting the columns reflect the degree of similarity between the respective gene expression patterns of the different liver spheroid states. As shown in FIG. 16A, a cluster analysis of the gene expression profiles of the liver spheroid states showed that, out of the liver spheroid states 1, 2, and 5-7 where NASH-induced liver spheroid were subjected to a potentially therapeutic treatment, the gene expression profile of the Primed exosome treated liver spheroid state most closely resembled the gene expression profile of the Healthy liver spheroid state. By contrast, the gene expression profile of the Naïve exosome treated liver spheroid state most closely resembled the gene expression profile of the Diseased liver spheroid state, indicating that whereas the primed exosomes were effective in at least partially reversing the NASH induction and improving the health of the NASH-induced liver spheroid, the naïve exosomes were ineffective in achieving such a result.

The gene expression levels of the liver spheroids in each of the different liver spheroid states were also represented and stored, respectively, as feature vectors (“spheroid state feature vectors”), with each element of a given spheroid state feature vector being a level of expression of one of the genes assayed in the microarray. The similarity (or lack of similarity) of the gene expression profile between different states was determined based on a measure of distance between pairs of feature vectors in an n-dimensional space, n being the number of genes represented in each of the feature vectors. Based on this distance analysis as well, it was found that, compared to other treatment conditions (namely the Naïve Exosome Concomitant condition, the Primed Exosome Concomitant condition, and the Naïve exosome treatment condition) spheroid state feature vector representing the Primed exosome treatment condition (in which NASH-induced liver spheroids were treated post induction with DMF-primed exosomes) was found to have the shortest Euclidean distance to spheroid feature vector representing the healthy liver spheroids (which were not subjected to NASH induction). In other words, treatment with exosomes from DMF-primed hBM-MSCs were shown to be the most effective treatment out of the treatments tested in returning the gene expression profile of the NASH-induced liver spheroids back to that of healthy liver spheroids.

FIG. 16B depicts a plot based on a principal component analysis of differentially expressed genes in healthy control liver spheroids, NASH-induced liver spheroids, and exosome treated NASH-induced liver spheroids. The figure shows a two-dimensional projection of the n-dimensional space comprising the various spheroid state feature vectors representing the same seven liver spheroid conditions described with respect to FIG. 16A: HGF, Naïve Exosome Treated. Diseased. Healthy, Primed exosome treated, Naïve exosome Concomitant, and Primed exosome Concomitant. The two-dimensional projection is based on principal component analysis. Induction of a NASH phenotype in the liver spheroids generated differentially expressed genes that shifted the gene profile down and to the left compared to healthy control gene profile. Based on the microarray-based transcriptomic analysis as visualized in the two-dimensional projection, it is readily apparent that treatment with naïve exosomes or with 40 ng/ml HGF was not effective in treating the NASH-induced liver, as shown in the figure, the gene expression profile of NASH-induced liver spheroid treated with naïve exosomes or HGF was substantially unchanged from untreated NASH-induced liver spheroids. By contrast, treatment of NASH-induced liver spheroids with exosomes derived from hBM-MSCs primed with Nrf2 activator DMF led to treated NASH-induced liver spheroids having a gene expression profile that shifted back upward and to the right, closer to the healthy control liver spheroids, demonstrating that the primed exosome treatment was substantially more effective that the other evaluated treatments (naïve exosomes and HGF) in reversing the NASH-induced genetic changes in the liver spheroids, and changing the gene expression profile of the NASH-induced liver spheroid to more closely resemble that of healthy liver spheroids.

In looking at the prophylactic effect of exosome treatment on NASH-induced spheroids, concomitant exosomes (primed and naïve) were used to treat NASH-induced liver spheroids. The treatment with concomitant exosomes whether naïve or primed significantly shifted the gene profile of differentially expressed genes to the right compared to diseased controls, indicating there was some prophylactic effect related to inhibition of fibrosis progression in NASH-induced liver spheroids with concomitant exosomes. There was some noticeable difference in differentially expressed genes between naïve concomitant exosome treatment on NASH-induced liver spheroids and primed concomitant exosome treatment on NASH-induced liver spheroids. That being said, concomitant exosome treatment, either with naïve or primed exosomes, was substantially less effective than the serial, non-concomitant treatment with primed exosomes in changing the gene expression profile of the NASH-induced liver spheroid to more closely resemble that of healthy liver spheroids.

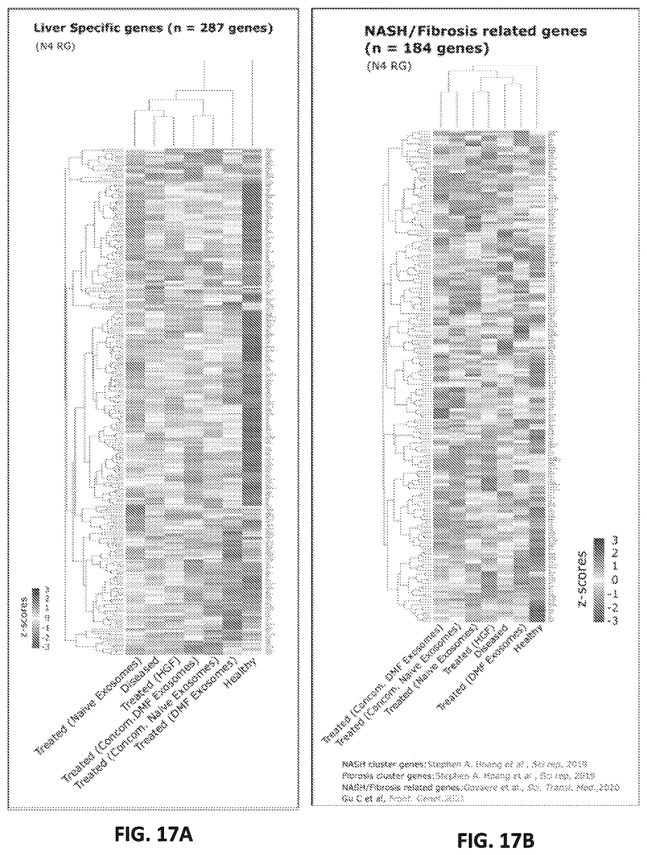

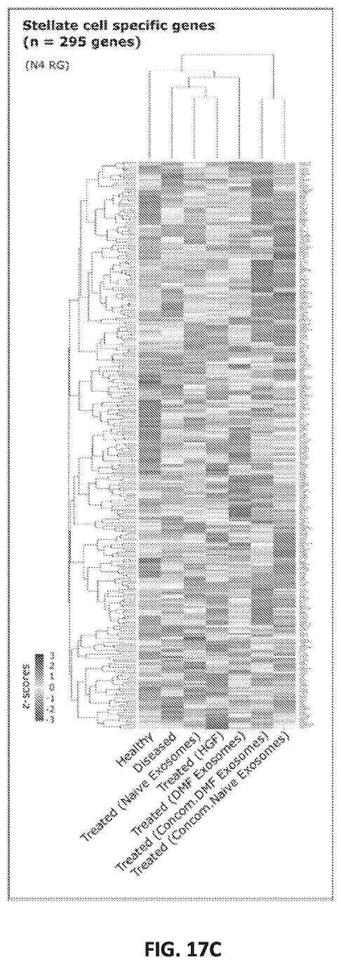

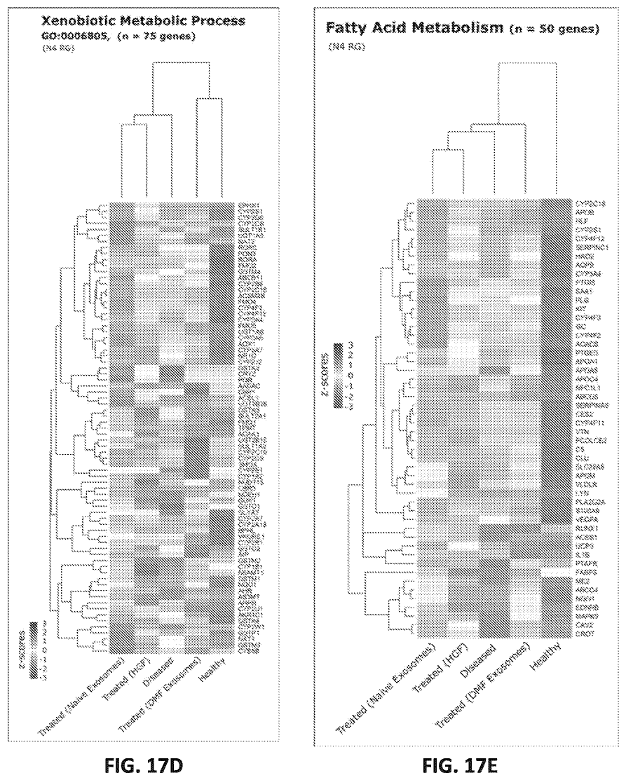

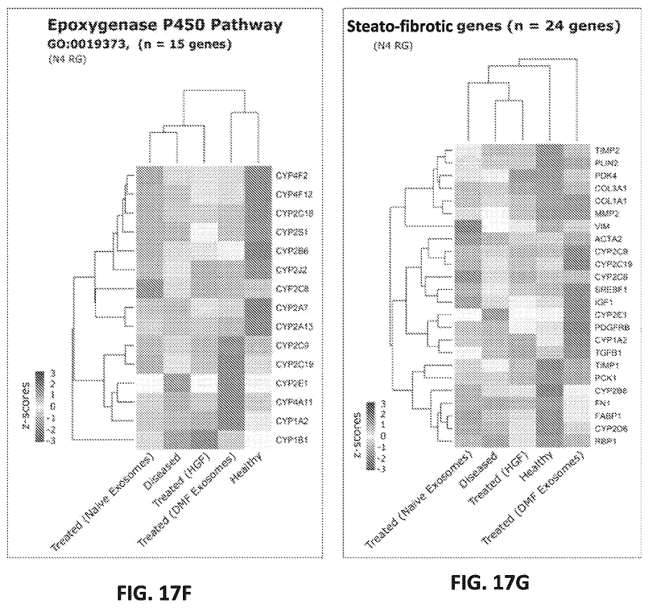

FIG. 17A depicts a heatmap 287 liver specific genes that are a subset of the genes assayed in the microarray study described above with respect to FIG. 16A. As shown in FIG. 17A, a cluster analysis of the gene expression profiles of the same set of liver spheroid states as shown in FIG. 16A showed that, out of the liver spheroid states where NASH-induced liver spheroids were subjected to a potentially therapeutic treatment, the gene expression profile of the Primed exosome treated liver spheroid state most closely resembled the gene expression profile of the Healthy liver spheroid state. By contrast, the gene expression profile of the HGF-treated liver spheroids most closely resembled the gene expression profile of the Diseased liver spheroid state, followed by the Naïve exosome treated liver spheroid state.

Making reference to FIG. 17B, repeating the analysis with different subsets of genes demonstrated that the DMF-primed exosome treatment was consistently the most effective in reversing NASH pathology. FIGS. 17B-17G shows the heatmaps with cluster analysis for the following gene subsets:

FIG. 17B: 184 NASH/Fibrosis-related genes (selection based on Hoang et al., Gene Expression Predicts Histological Severity and Reveals Distinct Molecular Profiles of Nonalcoholic Fatty Liver Disease, Scientific Reports 9 (12541) 2019, Govaere et al. Transcriptontic profiling across the nonalcoholic fatty liver disease spectrum reveals gene signatures for steatohepatitis and fibrosis. Science Translational Medicine 12(572). December 2020 and Gu C et al., Identification of Common Genes and Pathways in Eight Fibrosis Diseases, Frontiers in Genetics. January 2021).

FIG. 17C: 294 hepatic stellate cell-specific genes (selection of genes based on Payen et al., Single-cell RNA sequencing of human liver reveals hepatic stellate cell heterogeneity, JHEP Reports 3(3) Junc 2021).

FIG. 17D: 75 genes relevant to xenobiotic metabolic processes (selection based on Gene Ontology reference GO:0006805).

FIG. 17E: 50 genes relevant to fatty acid metabolism (selection based on Gene Ontology reference GO:0006631).

FIG. 17F: 15 genes relevant to the epoxygenase P450 pathway (selection based on Gene Ontology reference GO:0019373).

FIG. 17G: 24 genes relevant to steato-fibrosis, whose expression in diseased tissue associated with worsening of histologically defined NAFLD severity into steato-hepatitis and fibrosis in two independent cohorts of patients (selection of genes based on Govaere et al. Transcriptomic profiling across the nonalcoholic fatty liver disease spectrum reveals gene signatures for steatohepatitis and fibrosis. Science Translational Medicine 12(572), December 2020)

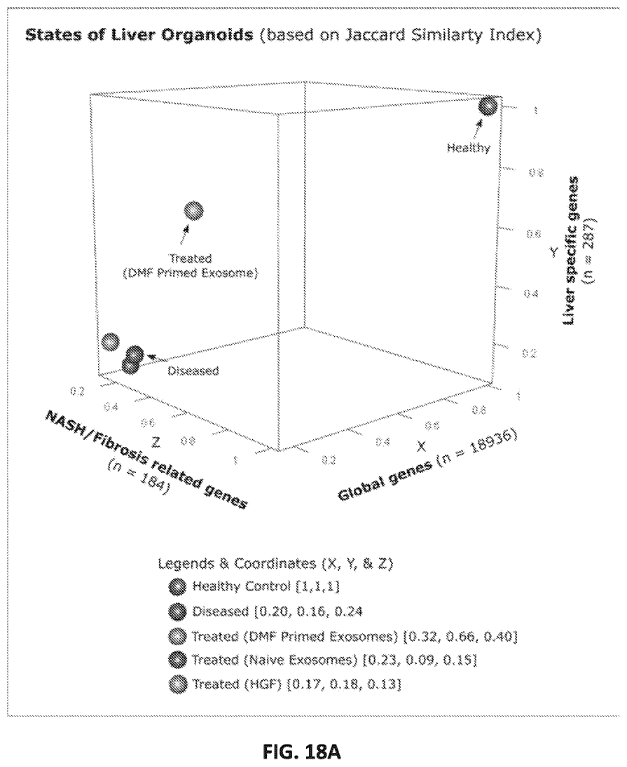

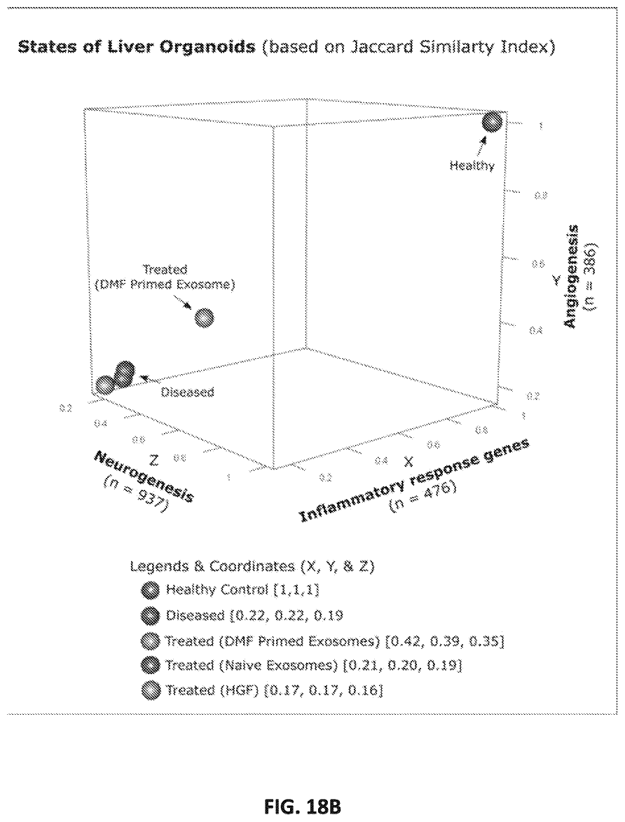

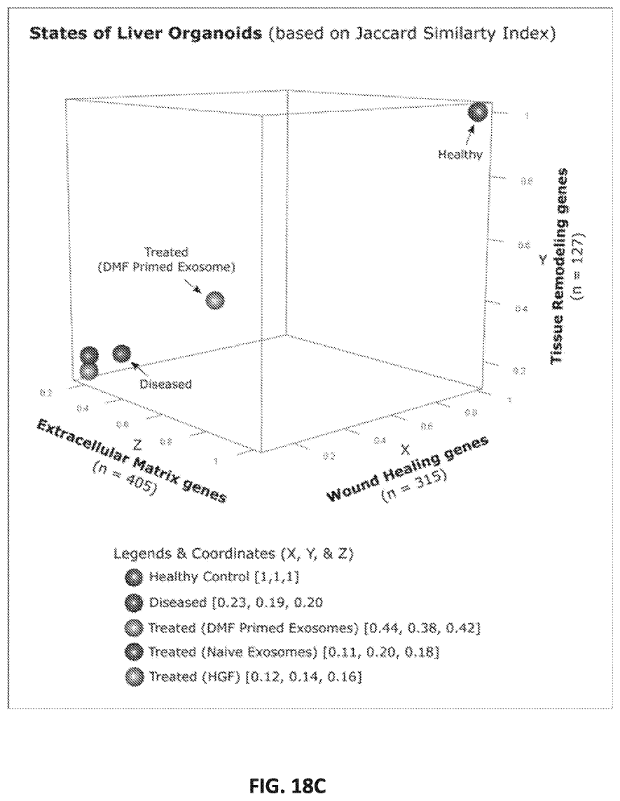

FIGS. 18A-C depict a graph of the states of liver spheroids before and after exosome treatment, the states including global genes, liver specific genes, and NASH/fibrosis related genes on the X, Y and Z axis, respectively. Each of FIGS. 18A-18C depicts a 3-dimensional projection a respective n-dimensional space comprising spheroid state feature vectors representing the following liver spheroid states: Heathy. Diseased (NASH-induced), HGF Treated, Naïve Exosome Treated, and Primed Exosome Treated. Each of FIGS. 18A-18C depicts a three-dimensional space depicted in each of FIGS. 18A-18C is a combination of three 2-dimensional projections, with each of the X, Y, and Z axes being based on a principle component analysis (PCA) of a matrix of Jaccard Similarity Index Scores of the subset of the genes assay in the microarray study described herein above (with some of the subjects being shown in FIGS. 17A-17G). In FIG. 18A, the X axis is based on the set of global genes with 18,936 genes, the Y axis is based on a set of 287 liver specific genes (depicted as a heatmap in FIG. 17A), and the Z axis is based on a set of 184 NASH/Fibrosis genes (depicted as a heatmap in FIG. 17B). In FIG. 18B, the X axis is based on a set of 476 inflammatory response genes based on GO:0006954, the Y axis is based on a set of 386 angiogenesis genes based on GO:0001525, and the Z axis is based on a set of 937 neurogenesis genes based on GO:0022008. In FIG. 18C, the X axis is based on a set of 315 wound healing genes based on GO:0042060, the Y axis is based on a set of 127 tissue remodelling genes (based on GO:0048771), and the Z axis is based on a set of 405 extracellular matrix genes based on GO:0031012. Each 2-dimensional projection, which is one side of the 3-dimensional space shown in FIGS. 18A-18C uses two out of the three axes provided in the respective 3-dimensional spaces.

In each projection, the Healthy liver spheroids state is assigned a coordinate of [1,1], and coordinates of the remaining states are determined based on a Jaccard Similarity index. As illustrated in each of FIGS. 18A-18C, DMF-primed exosome treatment of NASH-induced-liver spheroids induces a partial recovery, based on gene expression patterns, of the NASH-induced liver spheroids back towards a healthy state: the coordinate values of the DMF-primed exosome treated state is shifted forward on the Z axis, to the right on the X axis, and upward on the Y, away from the coordinate values for the diseased spheroids and towards the coordinate values of the healthy state. By contrast, Naïve exosome-treated NASH-induced liver spheroids, as well as HGF treated NASH-induced liver spheroids had coordinate values on all three axis that were substantially more similar to the diseased state than the DMF primed exosome treated state or the healthy state.

Based on the above results, it is expected that administration to a human subject with NASH of a therapeutically effective dose of DMF-primed exosomes will treat the NASH in the subject. It is also expected that the DMF-primed exosomes will be effective in treating NAFLD and NAFL. It is also expected that the DMF-primed exosomes will be effective in treating liver fibrosis.

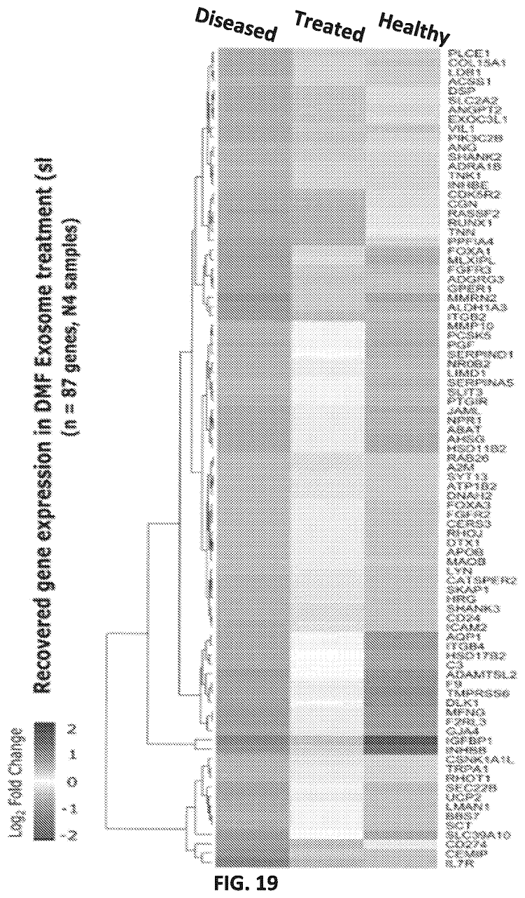

FIG. 19 depicts a heatmap of a shortlisted selection of 87 genes whose expression levels most robustly reversed from an expression level similar to the diseased state to an expression level similar to that of the healthy state after treatment by DMF-primed exosomes. These genes may be used as markers that for determining the state of health or NASH-induction not only in liver spheroids, but in liver tissue in vivo. These 87 genes include genes are a represented in various signaling pathways that are relevant to liver function as well as NAFLD and NASH disease progression, such as secretion (GL:0046903): cellular homeostasis (GO:0019725), wound healing (GO:0042050), lipid biosynthesis (GO:0008610). As such, the gene expression pattern of the liver spheroids during NASH induction and its treatment with the DMF-primed exosomes as described herein is shown to be mechanistically linked to clinical expressions of NAFLD and NASH in vivo as well as recovery from those conditions.

Out of the 87 genes, the following genes were determined to be particularly robust and useful as a maker for liver health in the liver spheroids as well as a maker for the recovery of said health through treatment with the DMF-primed exosomes: FOXA1, FOXA3, MMP10, FGFR2, FGFR3, ANGPT2, ANG, ATP1B, and ICAM2.

Table 12 shows the normalised values of gene expression in Log 2 for these nine genes.

TABLE 12

| | Healthy | Diseased (FFA+ | Treated (with DMF |
|---|---|---|---|
| Genes | Control | TGFβ1 Treated) | Primed Exosome) |
| FOXA1 | 6.915 | 5.631 | 6.695 |
| FOXA3 | 4.741 | 3.827 | 4.455 |
| MMP10 | 5.922 | 4.823 | 5.508 |
| FGFR2 | 6.643 | 5.752 | 6.382 |
| FGFR3 | 9.258 | 8.079 | 9.147 |
| ANGPT2 | 6.845 | 5.954 | 6.938 |
| ANG | 11.368 | 10.567 | 11.445 |
| ATP1B2 | 5.754 | 4.925 | 5.628 |
| ICAM2 | 8.715 | 7.690 | 8.507 |

### Advantages of the Present Disclosure

The present disclosure discloses a method of priming of MSCs derived from various tissue sources, such as bone marrow, adipose, umbilical cord, etc., with specific combination of inducers to activate certain pathways for the production of therapeutic exosomes with enriched factors, including anti-inflammatory, anti-fibrosis, pro-wound healing, angiogenesis (pro-/anti-), and re-innervation factors, for avascular and vascular regeneration of tissues.

The priming of MSCs is done with various priming agents (small molecules and macromolecules). The advantages of the present disclosure are as follows:
1. The present disclosure provides a selection of unique population of stem cells, such as, UC-MSCs/WJ-MSCs, based on the expression of signature set of markers to produce exosome with desired therapeutic effects, such as anti-inflammatory, anti-fibrosis, pro-wound healing, angiogenesis (pro-/anti-), and re-innervation properties.
2. The present disclosure also provides immortalization/engineering of human MSCs, using hTERT (human telomerase reverse transcriptase) to extend the doubling potential of MSCs (eMSCs) for facilitation of scalable and homogeneous production of cells and therapeutic exosomes.
3. The present disclosure provides a method that involves priming agents, such as Nrf2 activators, SIRT1 activators, all-trans retinoic acid (ATRA), CSSC-derived conditioned media, etc., which can be used alone or in combination thereof. The method deploying priming agents helps in obtaining enriched therapeutic grade exosomes with substantial enhancement in regenerative therapeutic efficacy.
4. The present disclosure also provides induced and activated exosomes for the treatment of inflammatory associated disorders including rheumatoid arthritis, systemic juvenile idiopathic arthritis, idiopathic pulmonary fibrosis, acute respiratory distress syndrome (ARDS), acute lung injury (ALI), pneumonia, bronchitis, chronic obstructive pulmonary disease (COPD), COVID-19, coronavirus class of infection, cystic fibrosis, hantavirus, influenza, tuberculosis, systemic lupus, myocardial infarction, osteoarthritis, non-alcoholic fatty liver disease (NASH), liver fibrosis, Mooren's ulcer, neurotrophic ulcer, and corneal keratitis (CK), dry eye disease ulcer, herpetic simplex keratitis, post-LASIK ectasia, postoperative corneal melts, post keratoprosthesis melts, corneal perforations, neurotrophic keratitis (NK), keratoconus Sjögren's syndrome, mucous membrane pemphigoid, Stevens-Johnson syndrome, chemical burns, and thermal burns.
5. The present disclosure also provides a cost-effective method as the amount of cell derived products of MSCs that are required to have a therapeutic effect in the animal model is ˜50 μg of protein or close to 10 billion particles. In order to perform physical, molecular and transcriptomic analysis, the strategy of scale up is the way forward which in turn reduces the cost involved significantly.
6. Overall, the present disclosure discloses a process of culturing, expansion, and priming of MSC with different priming agents to obtain primed MSC and a primed conditioned medium. The scalability of the process as described herein along with the fact that the process is a xeno-free process, therefore, gives a viable option of scalability for meeting the commercial requirements and also provides clinical grade end products in terms of primed MSCs, primed conditioned medium. The conditioned medium (CSSC-CM) or the primed conditioned medium is further processed to obtain clinical grade exosomes, secretome, and other cello-derived products which can be used for treating a condition selected from the group consisting of rheumatoid arthritis, systemic juvenile idiopathic arthritis, idiopathic pulmonary fibrosis, acute respiratory distress syndrome (ARDS), acute lung injury (ALI), pneumonia, bronchitis, chronic obstructive pulmonary disease (COPD). COVID-19, coronavirus class of infection, cystic fibrosis, hantavirus, influenza, tuberculosis, systemic lupus, myocardial infarction, osteoarthritis, non-alcoholic fatty liver disease (NASH), liver fibrosis, Mooren's ulcer, neurotrophic ulcer, and corneal keratitis (CK), dry eye disease ulcer, herpetic simplex keratitis, post-LASIK ectasia, postoperative corneal melts, post keratoprosthesis melts, corneal perforations, neurotrophic keratitis (NK), keratoconus Sjögren's syndrome, mucous membrane pemphigoid, Stevens-Johnson syndrome, chemical burns, and thermal burns. The exosome yield as per the present disclosure is scalable without impacting the production costs.

1-68. (canceled)

69. A method of generating a population of primed mesenchymal stem cell-derived exosomes, the method comprising:
(a) expanding a population of mesenchymal stem cells (MSCs) in culture;
(b) contacting the population of MSCs with a cell-derived conditioned medium derived from a population of cells different from the population of MSCs, and contacting the population of MSCs with at least one defined priming agent, to obtain a population of primed MSCs;
(c) growing the population of the primed MCSs in culture to produce a primed MSC-derived conditioned medium;
(d) collecting the primed MSC-conditioned medium; and
(e) purifying the exosomes from the primed MSC-conditioned medium.

70. The method of claim 69, wherein the population of MSCs are in contact with the cell-derived conditioned media from seeding until between about 60% and about 90% confluency, and the population of MSCs are in contact with the at least one defined priming agent starting from between about 60% and about 90% confluency.

71. The method of claim 69, wherein the population of MSCs are in contact with the cell-derived conditioned media from seeding until between about 60% and about 90% confluency, and are in contact with the at least one defined priming agent thereafter.

72. The method of claim 69, wherein the population of MSCs are in contact with the at least one defined priming agent for between about 12 hours and about 72 hours.

73. The method of claim 69, wherein:
the cell-derived conditioned medium from the different population of cells is a corneal stromal stem cell-derived conditioned medium.

74. The method of claim 69, wherein:
the at least one defined priming agent is a nuclear factor erythroid 2-related factor 2 (Nrf2) activator, a silencing information regulator factor 1 (SIRT1) activator, or an all-trans retinoic Acid (ATRA).

75. The method of claim 74, wherein the at least one defined priming agent is the Nrf2 activator.

76. The method of claim 75, wherein the Nrf2 activator is dimethyl fumarate (DMF) or 4 Octyl itaconate (4-OI).

77. The method of claim 69, wherein the population of MSCs is a population of bone marrow-derived MSCs (BM-MSCs), a population of umbilical cord-derived MSCs (UM-MSCs), a population of induced pluripotent stem cell (iPSC)-derived MSCs (iPSC-MSCs), or a population of Wharton's Jelly-derived MSCs (WJ-MSCs).

78. The method of claim 69, wherein the cell-derived conditioned medium is the corneal stem cell-derived conditioned medium, and the defined priming agent is DMF or 4 Octyl itaconate (4-OI).

79. A population of primed MSC-derived exosomes that are characterized by having, compared to unprimed MSC-derived exosomes:
(a) at least 2× higher expression level of sFLT1;
(b) an expression level of vascular endothelial growth factor (VEGF) that is a quarter or less of the expression in unprimed MSC-derived exosomes;
(c) at least 2× higher expression level of hepatic growth factor (HGF); and
(d) at least 3× higher expression level of nerve growth factor (NGF).

80. A method of treating a corneal defect, the method comprising administering to a corneal surface having the corneal defect a therapeutic dose of the population of exosomes of claim 79.

81. A method of generating a population of primed mesenchymal stem cell-derived exosomes, the method comprising:
(a) culturing a population of mesenchymal stem cells (MSCs) in a culture medium;
(b) contacting the population of MSCs with an nuclear factor erythroid 2-related factor 2 (Nrt2) activator to obtain a population of primed MSCs;
(c) growing the population of the primed MCSs in a collection medium, wherein the collection medium becomes enriched with exosomes produced by the primed MSCs, thereby producing a primed-MSC-derived conditioned medium;
(d) collecting the primed MSC-conditioned medium; and
(e) purifying the exosomes from the primed MSC-conditioned medium.

82. The method of claim 81, wherein the population of MSCs are grown in a first culture medium from seeding until between about 60% and about 90% confluency, then contacted with the Nrf2 activator.

83. The method of claim 81, wherein the population of MSCs are in contact with the Nrf2 activator for between about 12 hours and 72 hours.

84. The method of claim 81, wherein the Nrf2 activator is dimethyl fumarate (DMF) or 4 Octyl itaconate (4-OI).

85. The method of claim 84, wherein the Nrf2 activator is the DMF, and the DMF is present at a concentration of between about 50 μM and about 100 μM.

86. The method of claim 81, wherein the population of MSCs is a population of bone marrow MSCs (BM-MSCs), a population of umbilical cord-derived MSCs (UM-MSCs), a population of induced pluripotent stem cell (iPSC)-derived MSCs (iPSC-MSCs) or a population of Wharton's Jelly-derived MSCs (WJ-MSCs).

87. A population of primed MSC-derived exosomes that are characterized by having one or more of, as compared to unprimed MSC-derived exosomes:
(a) at least 1.2× higher expression level of HGF; and
(b) at least 2× higher expression level of NGF.

88. A method of treating a liver condition, the method comprising administering to a subject having the liver condition a therapeutic amount of the population of exosomes of claim 87.
